SEQUENCE LISTING (104 sequences)

SEQ ID NO: 1 - protein, 323 aa, Homo sapiens
QNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 2 - protein, 348 aa, Artificial Sequence
  CTGF-his
MEFGLSWLFLVAILKGVQCQNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMAHHHHHH

SEQ ID NO: 3 - protein, 575 aa, Artificial Sequence
  mCTGF-Fc
MEFGLSWLFLVAILKGVQCQDCSAQCQCAAEAAPHCPAGVSLVLDGCGCCRVCAKQLGELCTERDPCDPHKGLFCDFGSPANRKIGVCTAKDGAPCVFGGSVYRSGESFQSSCKYQCTCLDGAVGCVPLCSMDVRLPSPDCPFPRRVKLPGKCCEEWVCDEPKDRTAVGPALAAYRLEDTFGPDPTMMRANCLVQTTEWSACSKTCGMGISTRVTNDNTFCRLEKQSRLCMVRPCEADLEENIKKGKKCIRTPKIAKPVKFELSGCTSVKTYRAKFCGVCTDGRCCTPHRTTTLPVEFKCPDGEIMKKNMMFIKTCACHYNCPGDNDIFESLYYRKMYGDMAEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 4 - protein, 348 aa, Artificial Sequence
  mouse CTGF protein-His tag
MEFGLSWLFLVAILKGVQCQDCSAQCQCAAEAAPHCPAGVSLVLDGCGCCRVCAKQLGELCTERDPCDPHKGLFCDFGSPANRKIGVCTAKDGAPCVFGGSVYRSGESFQSSCKYQCTCLDGAVGCVPLCSMDVRLPSPDCPFPRRVKLPGKCCEEWVCDEPKDRTAVGPALAAYRLEDTFGPDPTMMRANCLVQTTEWSACSKTCGMGISTRVTNDNTFCRLEKQSRLCMVRPCEADLEENIKKGKKCIRTPKIAKPVKFELSGCTSVKTYRAKFCGVCTDGRCCTPHRTTTLPVEFKCPDGEIMKKNMMFIKTCACHYNCPGDNDIFESLYYRKMYGDMAHHHHHH

SEQ ID NO: 5 - protein, 574 aa, Artificial Sequence
  cynomolgus CTGF-Fc
MEFGLSWLFLVAILKGVQCQNCSGPCRCPAEQAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSVKTYRAKFCGVCTDGRCCTPHRTTTLPVEFKCPDGEVMKKNMMFIKTCACHYNCPGDNDIFESLYYRKMYGDMAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 116 aa, Mus musculus
EVQLVESGGGLVQPEGSLKLSCAASGFSFNTYAMNWVRQAPGKGLEWVARIRTKSNNYATYYADSVKDRFTISRDDSESMLYLQMNNLKTEDTAMYYCVETGFAYWDQGTLVTVSA

SEQ ID NO: 7 - protein, 106 aa, Mus musculus
QIVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELK

SEQ ID NO: 8 - protein, 114 aa, Mus musculus
QVQLKQSGPGLVQPSQSLSITCTVSGFSLTTFGVHWIRQSPGKGLEWLGVIWRRGGTDYNAAFMSRLSITKDNSRSHVFFKMTSLQTDDSAIYYCARDGGFDYWGQGTTLTVSS

SEQ ID NO: 9 - protein, 109 aa, Mus musculus
QAVVTQESALTTSPGGTVILTCRSSIGAVTTSNYANWVQEKPDHLFTGLIGGTSNRAPGVPVRFSGSLIGDKAALTITGAQAEDDAMYFCALWYSTHYVFGGGTKVTVL

SEQ ID NO: 10 - protein, 5 aa, Mus musculus
TYAMN

SEQ ID NO: 11 - protein, 19 aa, Mus musculus
RIRTKSNNYATYYADSVKD

SEQ ID NO: 12 - protein, 5 aa, Mus musculus
TGFAY

SEQ ID NO: 13 - protein, 10 aa, Mus musculus
SASSSVSYMH

SEQ ID NO: 14 - protein, 7 aa, Mus musculus
STSNLAS

SEQ ID NO: 15 - protein, 9 aa, Mus musculus
QQRSSYPLT

SEQ ID NO: 16 - protein, 5 aa, Mus musculus
TFGVH

SEQ ID NO: 17 - protein, 16 aa, Mus musculus
VIWRRGGTDYNAAFMS

SEQ ID NO: 18 - protein, 6 aa, Mus musculus
DGGFDY

SEQ ID NO: 19 - protein, 14 aa, Mus musculus
RSSIGAVTTSNYAN

SEQ ID NO: 20 - protein, 7 aa, Mus musculus
GTSNRAP

SEQ ID NO: 21 - protein, 9 aa, Mus musculus
ALWYSTHYV

SEQ ID NO: 22 - protein, 106 aa, Artificial Sequence
  Hu147-VL1
EIVLTQSPATLSLSPGERATLSCSASSSVSYMHWFQQKPGQSPRLLIYSTSNLASGIPARFSGSGSGTDYTLTISSLEPEDFAVYYCQQRSSYPLTFGQGTKLEIK

SEQ ID NO: 23 - protein, 106 aa, Artificial Sequence
  Hu147-VL2
EIVLTQSPATLSLSPGERATLSCSASSSVSYMHWFQQKPGQSPKLLIYSTSNLASGIPARFSGSGSGTDYTLTISSLEPEDFAVYYCQQRSSYPLTFGQGTKLEIK

SEQ ID NO: 24 - protein, 106 aa, Artificial Sequence
  Hu147-VL3
EIVLTQSPATLSLSPGERATLSCSASSSVSYMHWFQQKPGQSPKLWIYSTSNLASGVPARFSGSGSGTDYTLTISSLEPEDFAVYYCQQRSSYPLTFGQGTKLEIK

SEQ ID NO: 25 - protein, 106 aa, Artificial Sequence
  Hu147-VL4
DIQMTQSPSSLSASVGDRVTITCSASSSVSYMHWFQQKPGKSPKLLIYSTSNLASGVPSRFSGSGSGTDYTLTISSLQPEDFATYYCQQRSSYPLTFGQGTKLEIK

SEQ ID NO: 26 - protein, 106 aa, Artificial Sequence
  Hu147-VL5
DIQLTQSPSSLSASVGDRVTITCSASSSVSYMHWFQQKPGKSPKLWIYSTSNLASGVPSRFSGSGSGTDYTLTISSLQPEDFATYYCQQRSSYPLTFGQGTKLEIK

SEQ ID NO: 27 - protein, 116 aa, Artificial Sequence
  Hu147-VH1
EVQLVESGGGLVQPGGSLRLSCAASGFTFSTYAMNWVRQAPGKGLEWVGRIRTKSNNYATYYADSVKDRFTISRDDSKNSLYLQMNSLKTEDTAVYYCVETGFAYWDQGTLVTVSS

SEQ ID NO: 28 - protein, 116 aa, Artificial Sequence
  Hu147-VH2
EVQLVESGGGLVQPGGSLRLSCAASGFTFNTYAMNWVRQAPGKGLEWVARIRTKSNNYATYYADSVKDRFTISRDDSKNSLYLQMNSLKTEDTAVYYCVETGFAYWDQGTLVTVSS

SEQ ID NO: 29 - protein, 116 aa, Artificial Sequence
  Hu147-VH3
EVQLVESGGGLVQPGGSLRLSCAASGFSFNTYAMNWVRQAPGKGLEWVARIRTKSNNYATYYADSVKDRFTISRDDSESSLYLQMNSLKTEDTAVYYCVETGFAYWDQGTLVTVSS

SEQ ID NO: 30 - protein, 109 aa, Artificial Sequence
  Hu164-VL7
ETVVTQEPSLTVSPGGTVTLTCRSSIGAVTTSNYANWVQQKPGQAFRGLIGGTSNRAPWTPARFSGSLLGGKAALTLSGVQPEDEAEYYCALWYSTHYVFGGGTKLTVL

SEQ ID NO: 31 - protein, 109 aa, Artificial Sequence
  Hu164-VL8
ETVVTQEPSFSVSPGGTVTLTCRSSIGAVTTSNYANWVQQTPGQAFRGLIGGTSNRAPGVPDRFSGSILGNKAALTITGAQADDESDYYCALWYSTHYVFGGGTKLTVL

SEQ ID NO: 32 - protein, 109 aa, Artificial Sequence
  Hu164-VL9
ESVVTQPPSVSGAPGQRVTISCRSSIGAVTTSNYANWVQQLPGTAFKGLIGGTSNRAPGVPDRFSGSKSGTSASLAITGLQAEDEADYYCALWYSTHYVFGGGTKLTVL

SEQ ID NO: 33 - protein, 114 aa, Artificial Sequence
  Hu164-VH5
EVTLKESGPVLVKPTETLTLTCTVSGFSLSTFGVHWIRQPPGKALEWLAVIWRRGGTDYNAAFMSRLTISKDTSKSQVVLTMTNMDPVDTATYYCARDGGFDYWGQGTTVTVSS

SEQ ID NO: 34 - protein, 114 aa, Artificial Sequence
  Hu164-VH6
EVTLKESGPVLVKPTETLTLTCTVSGFSLSTFGVHWIRQPPGKGLEWLGVIWRRGGTDYNAAFMSRLTISKDTSKSQVVFTMTNMDPVDTATYYCARDGGFDYWGQGTTVTVSS

SEQ ID NO: 35 - protein, 114 aa, Artificial Sequence
  Hu164-VH7
EVQLQESGPGLVKPSQTLSLTCTVSGFSISTFGVHWIRQHPGKGLEWIGVIWRRGGTDYNAAFMSRVTISKDTSKNQVSLKLSSVTAADTAVYYCARDGGFDYWGQGTTVTVSS

SEQ ID NO: 36 - protein, 114 aa, Artificial Sequence
  Hu164-VH8
EVQLQESGPGLVKPSQTLSLTCTVSGFSISTFGVHWIRQHPGKGLEWLGVIWRRGGTDYNAAFMSRLTISKDTSKNQVSFKLSSVTAADTAVYYCARDGGFDYWGQGTTVTVSS

SEQ ID NO: 37 - protein, 330 aa, Artificial Sequence [sequence omitted]

SEQ ID NO: 38 - protein, 330 aa, Artificial Sequence
  heavy chain variable region y
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 107 aa, Artificial Sequence [sequence omitted]

SEQ ID NO: 40 - protein, 105 aa, Artificial Sequence
  humanized antibody light chain lambda constant region
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 41 - protein, 446 aa, Artificial Sequence
  Ch147 heavy chain
EVQLVESGGGLVQPEGSLKLSCAASGFSFNTYAMNWVRQAPGKGLEWVARIRTKSNNYATYYADSVKDRFTISRDDSESMLYLQMNNLKTEDTAMYYCVETGFAYWDQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 213 aa, Artificial Sequence
  Ch147 light chain
QIVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 446 aa, Artificial Sequence
  Hu147-11wk heavy chain
EVQLVESGGGLVQPGGSLRLSCAASGFTFSTYAMNWVRQAPGKGLEWVGRIRTKSNNYATYYADSVKDRFTISRDDSKNSLYLQMNSLKTEDTAVYYCVETGFAYWDQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 446 aa, Artificial Sequence
  Hu147-21wk heavy chain
EVQLVESGGGLVQPGGSLRLSCAASGFTFNTYAMNWVRQAPGKGLEWVARIRTKSNNYATYYADSVKDRFTISRDDSKNSLYLQMNSLKTEDTAVYYCVETGFAYWDQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 446 aa, Artificial Sequence
  Hu147-31wk heavy chain
EVQLVESGGGLVQPGGSLRLSCAASGFSFNTYAMNWVRQAPGKGLEWVARIRTKSNNYATYYADSVKDRFTISRDDSESSLYLQMNSLKTEDTAVYYCVETGFAYWDQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 446 aa, Artificial Sequence
  Hu147-11yk heavy chain
EVQLVESGGGLVQPGGSLRLSCAASGFTFSTYAMNWVRQAPGKGLEWVGRIRTKSNNYATYYADSVKDRFTISRDDSKNSLYLQMNSLKTEDTAVYYCVETGFAYWDQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 446 aa, Artificial Sequence
  Hu147-21yk heavy chain
EVQLVESGGGLVQPGGSLRLSCAASGFTFNTYAMNWVRQAPGKGLEWVARIRTKSNNYATYYADSVKDRFTISRDDSKNSLYLQMNSLKTEDTAVYYCVETGFAYWDQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 446 aa, Artificial Sequence
  Hu147-31yk heavy chain
EVQLVESGGGLVQPGGSLRLSCAASGFSFNTYAMNWVRQAPGKGLEWVARIRTKSNNYATYYADSVKDRFTISRDDSESSLYLQMNSLKTEDTAVYYCVETGFAYWDQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 213 aa, Artificial Sequence
  Hu147-11wk light chain
EIVLTQSPATLSLSPGERATLSCSASSSVSYMHWFQQKPGQSPRLLIYSTSNLASGIPARFSGSGSGTDYTLTISSLEPEDFAVYYCQQRSSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 213 aa, Artificial Sequence
  Hu147-12wk light chain
EIVLTQSPATLSLSPGERATLSCSASSSVSYMHWFQQKPGQSPKLLIYSTSNLASGIPARFSGSGSGTDYTLTISSLEPEDFAVYYCQQRSSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 213 aa, Artificial Sequence
  Hu147-13wk light chain
EIVLTQSPATLSLSPGERATLSCSASSSVSYMHWFQQKPGQSPKLWIYSTSNLASGVPARFSGSGSGTDYTLTISSLEPEDFAVYYCQQRSSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 213 aa, Artificial Sequence
  Hu147-14wk light chain
DIQMTQSPSSLSASVGDRVTITCSASSSVSYMHWFQQKPGKSPKLLIYSTSNLASGVPSRFSGSGSGTDYTLTISSLQPEDFATYYCQQRSSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 213 aa, Artificial Sequence
  Hu147-15wk light chain
DIQLTQSPSSLSASVGDRVTITCSASSSVSYMHWFQQKPGKSPKLWIYSTSNLASGVPSRFSGSGSGTDYTLTISSLQPEDFATYYCQQRSSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 444 aa, Artificial Sequence
  Ch164 heavy chain
QVQLKQSGPGLVQPSQSLSITCTVSGFSLTTFGVHWIRQSPGKGLEWLGVIWRRGGTDYNAAFMSRLSITKDNSRSHVFFKMTSLQTDDSAIYYCARDGGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 214 aa, Artificial Sequence
  Ch164 light chain
QAVVTQESALTTSPGGTVILTCRSSIGAVTTSNYANWVQEKPDHLFTGLIGGTSNRAPGVPVRFSGSLIGDKAALTITGAQAEDDAMYFCALWYSTHYVFGGGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 56 - protein, 444 aa, Artificial Sequence
  Hu164-57wl heavy chain
EVTLKESGPVLVKPTETLTLTCTVSGFSLSTFGVHWIRQPPGKALEWLAVIWRRGGTDYNAAFMSRLTISKDTSKSQVVLTMTNMDPVDTATYYCARDGGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 444 aa, Artificial Sequence
  Hu164-67wl heavy chain
EVTLKESGPVLVKPTETLTLTCTVSGFSLSTFGVHWIRQPPGKGLEWLGVIWRRGGTDYNAAFMSRLTISKDTSKSQVVFTMTNMDPVDTATYYCARDGGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 444 aa, Artificial Sequence
  Hu164-77wl heavy chain
EVQLQESGPGLVKPSQTLSLTCTVSGFSISTFGVHWIRQHPGKGLEWIGVIWRRGGTDYNAAFMSRVTISKDTSKNQVSLKLSSVTAADTAVYYCARDGGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 444 aa, Artificial Sequence
  Hu164-87wl heavy chain
EVQLQESGPGLVKPSQTLSLTCTVSGFSISTFGVHWIRQHPGKGLEWLGVIWRRGGTDYNAAFMSRLTISKDTSKNQVSFKLSSVTAADTAVYYCARDGGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 444 aa, Artificial Sequence
  Hu164-57yl heavy chain
EVTLKESGPVLVKPTETLTLTCTVSGFSLSTFGVHWIRQPPGKALEWLAVIWRRGGTDYNAAFMSRLTISKDTSKSQVVLTMTNMDPVDTATYYCARDGGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 444 aa, Artificial Sequence
  Hu164-67yl heavy chain
EVTLKESGPVLVKPTETLTLTCTVSGFSLSTFGVHWIRQPPGKGLEWLGVIWRRGGTDYNAAFMSRLTISKDTSKSQVVFTMTNMDPVDTATYYCARDGGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 444 aa, Artificial Sequence
  Hu164-77yl heavy chain
EVQLQESGPGLVKPSQTLSLTCTVSGFSISTFGVHWIRQHPGKGLEWIGVIWRRGGTDYNAAFMSRVTISKDTSKNQVSLKLSSVTAADTAVYYCARDGGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 444 aa, Artificial Sequence
  Hu164-87yl heavy chain
EVQLQESGPGLVKPSQTLSLTCTVSGFSISTFGVHWIRQHPGKGLEWLGVIWRRGGTDYNAAFMSRLTISKDTSKNQVSFKLSSVTAADTAVYYCARDGGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 214 aa, Artificial Sequence
  Hu164-57wl light chain
ETVVTQEPSLTVSPGGTVTLTCRSSIGAVTTSNYANWVQQKPGQAFRGLIGGTSNRAPWTPARFSGSLLGGKAALTLSGVQPEDEAEYYCALWYSTHYVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 65 - protein, 214 aa, Artificial Sequence
  Hu164-58wl light chain
ETVVTQEPSFSVSPGGTVTLTCRSSIGAVTTSNYANWVQQTPGQAFRGLIGGTSNRAPGVPDRFSGSILGNKAALTITGAQADDESDYYCALWYSTHYVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 66 - protein, 214 aa, Artificial Sequence
  Hu164-59wl light chain
ESVVTQPPSVSGAPGQRVTISCRSSIGAVTTSNYANWVQQLPGTAFKGLIGGTSNRAPGVPDRFSGSKSGTSASLAITGLQAEDEADYYCALWYSTHYVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 67 - protein, 450 aa, Artificial Sequence
  mAb1 heavy chain
EGQLVQSGGGLVHPGGSLRLSCAGSGFTFSSYGMHWVRQAPGKGLEWVSGIGTGGGTYSTDSVKGRFTISRDNAKNSLYLQMNSLRAEDMAVYYCARGDYYGSGSFFDCWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 214 aa, Artificial Sequence
  mAb1 light chain
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 122 aa, Artificial Sequence
  mab95 VH
EVLLQQSGPVLVKPGPSVKISCKASGFTFTNYDIHWVKQSHGKSLEWIGLVYPYNGGTAYNQKFKDKATLTFDTSSSTAYMELNSLTSEDSAVYYCARWGLIPGTTSYFDVWGTGTTVTVSS

SEQ ID NO: 70 - protein, 106 aa, Artificial Sequence
  mab95 VL
QIVLSQSPPILSASPGEKVTMTCRASSSVSYIHWYQQKPRSSPKPWIYATSNLASGVPARFSGSGSGTSYSLTISRVEAEDAATYYCQQWNSNPWTFGGGTRLEIK

SEQ ID NO: 71 - protein, 5 aa, Artificial Sequence
  mab95 HCDR1
NYDIH

SEQ ID NO: 72 - protein, 17 aa, Artificial Sequence
  mab95 HCDR2
LVYPYNGGTAYNQKFKD

SEQ ID NO: 73 - protein, 13 aa, Artificial Sequence
  mab95 HCDR3
WGLIPGTTSYFDV

SEQ ID NO: 74 - protein, 10 aa, Artificial Sequence
  mab95 LCDR1
RASSSVSYIH

SEQ ID NO: 75 - protein, 7 aa, Artificial Sequence
  mab95 LCDR2
ATSNLAS

SEQ ID NO: 76 - protein, 9 aa, Artificial Sequence
  mab95 LCDR3
QQWNSNPWT

SEQ ID NO: 77 - protein, 106 aa, Artificial Sequence
  Hu95-VL1
EIVLTQSPATLSLSPGERATLSCRASSSVSYIHWYQQKPGQAPRPWIYATSNLASGIPARFSGSGSGTDYTLTISRLEPEDFAVYYCQQWNSNPWTFGGGTKVEIK

SEQ ID NO: 78 - protein, 106 aa, Artificial Sequence
  Hu95-VL2
EIVLTQSPATLSLSPGERATLSCRASSSVSYIHWYQQKPGQSPRPWIYATSNLASGVPARFSGSGSGTSYTLTISRLEPEDFAVYYCQQWNSNPWTFGGGTKVEIK

SEQ ID NO: 79 - protein, 105 aa, Artificial Sequence
  Hu95-VL3
ESVLTQPPSVSGAPGQRVTISCRASSSVSYIHWYQQLPGTAPKPWIYATSNLASGVPDRFSGSKSGTSYSLAITGLQAEDEADYYCQQWNSNPWTFGGGTKVEIK

SEQ ID NO: 80 - protein, 105 aa, Artificial Sequence
  Hu95-VL4
EIVLTQPPSVSGAPGQRVTISCRASSSVSYIHWYQQLPGTSPKPWIYATSNLASGVPDRFSGSKSGTSYSLAITGLQAEDEADYYCQQWNSNPWTFGGGTKVEIK

SEQ ID NO: 81 - protein, 122 aa, Artificial Sequence
  Hu95-VH1
EVQLVQSGAEVKKPGASVKVSCKASGFTFTNYDIHWVRQAPGQRLEWMGLVYPYNGGTAYNQKFKDRVTLTFDTSASTAYMELSSLRSEDTAVYYCARWGLIPGTTSYFDVWGQGTTVTVSS

SEQ ID NO: 82 - protein, 122 aa, Artificial Sequence
  Hu95-VH2
EVQLVQSGAEVKKPGASVKVSCKASGFTFTNYDIHWVRQAPGQRLEWIGLVYPYNGGTAYNQKFKDRATLTFDTSASTAYMELSSLRSEDTAVYYCARWGLIPGTTSYFDVWGQGTTVTVSS

SEQ ID NO: 83 - protein, 122 aa, Artificial Sequence
  Hu95-VH3
EVQLVQSGAEVKKPGASVKVSCKASGFTFTNYDIHWVKQAPGQRLEWMGLVYPYNGGTAYNQKFKDKVTLTFDTSASTAYMELSSLRSEDTAVYYCARWGLIPGTTSYFDVWGQGTTVTVSS

SEQ ID NO: 84 - protein, 122 aa, Artificial Sequence
  Hu95-VH4
EVQLVQSGAEVKKPGASVKVSCKASGFTFTNYDIHWVKQAPGQRLEWIGLVYPYNGGTAYNQKFKDKATLTFDTSASTAYMELSSLRSEDTAVYYCARWGLIPGTTSYFDVWGQGTTVTVSS

SEQ ID NO: 85 - protein, 122 aa, Artificial Sequence
  Hu95-VH5
EVQLVQSGAEVKKPGASVKVSCRASGFTFTNYAIHWVKQAPGQRLEWMGLVYPYTGGTAYNQKFKDKVTLTFDTSASTAYMELSSLRSEDTAVYYCARWGMIPGTNSYFDVWGQGTTVTVSS

SEQ ID NO: 86 - protein, 452 aa, Artificial Sequence
  Ch95 heavy chain
EVLLQQSGPVLVKPGPSVKISCKASGFTFTNYDIHWVKQSHGKSLEWIGLVYPYNGGTAYNQKFKDKATLTFDTSSSTAYMELNSLTSEDSAVYYCARWGLIPGTTSYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 213 aa, Artificial Sequence
  Ch95 light chain
QIVLSQSPPILSASPGEKVTMTCRASSSVSYIHWYQQKPRSSPKPWIYATSNLASGVPARFSGSGSGTSYSLTISRVEAEDAATYYCQQWNSNPWTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 452 aa, Artificial Sequence
  Hu95-11wk  heavy chain
EVQLVQSGAEVKKPGASVKVSCKASGFTFTNYDIHWVRQAPGQRLEWMGLVYPYNGGTAYNQKFKDRVTLTFDTSASTAYMELSSLRSEDTAVYYCARWGLIPGTTSYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 452 aa, Artificial Sequence
  Hu95-21wk  heavy chain
EVQLVQSGAEVKKPGASVKVSCKASGFTFTNYDIHWVRQAPGQRLEWIGLVYPYNGGTAYNQKFKDRATLTFDTSASTAYMELSSLRSEDTAVYYCARWGLIPGTTSYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 452 aa, Artificial Sequence
  Hu95-31wk  heavy chain
EVQLVQSGAEVKKPGASVKVSCKASGFTFTNYDIHWVKQAPGQRLEWMGLVYPYNGGTAYNQKFKDKVTLTFDTSASTAYMELSSLRSEDTAVYYCARWGLIPGTTSYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 452 aa, Artificial Sequence
  Hu95-41wk  heavy chain
EVQLVQSGAEVKKPGASVKVSCKASGFTFTNYDIHWVKQAPGQRLEWIGLVYPYNGGTAYNQKFKDKATLTFDTSASTAYMELSSLRSEDTAVYYCARWGLIPGTTSYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 452 aa, Artificial Sequence
  Hu95-51wk  heavy chain
EVQLVQSGAEVKKPGASVKVSCRASGFTFTNYAIHWVKQAPGQRLEWMGLVYPYTGGTAYNQKFKDKVTLTFDTSASTAYMELSSLRSEDTAVYYCARWGMIPGTNSYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 452 aa, Artificial Sequence
  Hu95-11yk  heavy chain
EVQLVQSGAEVKKPGASVKVSCKASGFTFTNYDIHWVRQAPGQRLEWMGLVYPYNGGTAYNQKFKDRVTLTFDTSASTAYMELSSLRSEDTAVYYCARWGLIPGTTSYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 452 aa, Artificial Sequence
  Hu95-21yk  heavy chain
EVQLVQSGAEVKKPGASVKVSCKASGFTFTNYDIHWVRQAPGQRLEWIGLVYPYNGGTAYNQKFKDRATLTFDTSASTAYMELSSLRSEDTAVYYCARWGLIPGTTSYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 452 aa, Artificial Sequence
  Hu95-31yk
EVQLVQSGAEVKKPGASVKVSCKASGFTFTNYDIHWVKQAPGQRLEWMGLVYPYNGGTAYNQKFKDKVTLTFDTSASTAYMELSSLRSEDTAVYYCARWGLIPGTTSYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 452 aa, Artificial Sequence
  Hu95-41yk  heavy chain
EVQLVQSGAEVKKPGASVKVSCKASGFTFTNYDIHWVKQAPGQRLEWIGLVYPYNGGTAYNQKFKDKATLTFDTSASTAYMELSSLRSEDTAVYYCARWGLIPGTTSYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 452 aa, Artificial Sequence
  Hu95-51yk  heavy chain
EVQLVQSGAEVKKPGASVKVSCRASGFTFTNYAIHWVKQAPGQRLEWMGLVYPYTGGTAYNQKFKDKVTLTFDTSASTAYMELSSLRSEDTAVYYCARWGMIPGTNSYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 213 aa, Artificial Sequence
  Hu95-11wk light chain
EIVLTQSPATLSLSPGERATLSCRASSSVSYIHWYQQKPGQAPRPWIYATSNLASGIPARFSGSGSGTDYTLTISRLEPEDFAVYYCQQWNSNPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 213 aa, Artificial Sequence
  Hu95-12wk light chain
EIVLTQSPATLSLSPGERATLSCRASSSVSYIHWYQQKPGQSPRPWIYATSNLASGVPARFSGSGSGTSYTLTISRLEPEDFAVYYCQQWNSNPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 212 aa, Artificial Sequence
  Hu95-13wk light chain
ESVLTQPPSVSGAPGQRVTISCRASSSVSYIHWYQQLPGTAPKPWIYATSNLASGVPDRFSGSKSGTSYSLAITGLQAEDEADYYCQQWNSNPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 212 aa, Artificial Sequence
  Hu95-14wk light chain
EIVLTQPPSVSGAPGQRVTISCRASSSVSYIHWYQQLPGTSPKPWIYATSNLASGVPDRFSGSKSGTSYSLAITGLQAEDEADYYCQQWNSNPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 5 aa, Artificial Sequence
  Hu95-VH5 HCDR1
NYAIH

SEQ ID NO: 103 - protein, 17 aa, Artificial Sequence
  Hu95-VH5 HCDR2
LVYPYTGGTAYNQKFKD

SEQ ID NO: 104 - protein, 13 aa, Artificial Sequence
  Hu95-VH5 HCDR3
WGMIPGTNSYFDV

(19) United States
(12) Patent Application Publication
(10) Pub. No.: US 2022/0324953 A1
(43) Pub. Date: Oct. 13, 2022

(54) ANTI-CONNECTIVE TISSUE GROWTH FACTOR ANTIBODY AND APPLICATION THEREOF

(71) Applicants: JIANGSU HENGRUI MEDICINE CO., LTD., Lianyungang (CN); SHANGHAI HENGRUI PHARMACEUTICAL CO., LTD., Shanghai (CN)

(72) Inventors: Yayuan FU, Shanghai (CN); Xiaoli MA, Shanghai (CN); Hu GE, Shanghai (CN); Weikang TAO, Shanghai (CN)

(21) Appl. No.: 17/615,801

(22) PCT Filed: Jun. 3, 2020
(86) PCT No.: PCT/CN2020/094136

§ 371 Date: Dec. 1, 2021

(30) Foreign Application Priority Data
Jun. 4, 2019 (CN) 201910480169.4

(51) Int. Cl. C07K 16/22; C12N 15/63; G01N 33/53

(52) U.S. Cl. CPC C07K 16/22; C12N 15/63; G01N 33/53; C07K 2317/24; C07K 2317/33; C07K 2317/73; C07K 2317/76; C07K 2317/92; C07K 2317/565

(57) ABSTRACT

Provided are an anti-CTGF antibody comprising variable regions of a light chain and a heavy chain and a use thereof as a drug. The antibody may be used to prepare drugs for treating CTGF-related diseases or conditions.

The present application claims the priority of the Chinese patent application No.201910480169.4 (title: Anti-Connective Tissue Growth Factor Antibody and Application Thereof, Priority Date: Jun. 4, 2019).

### FIELD OF THE INVENTION

The present disclosure belongs to the field of biomedicine, and specifically relates to antibodies binding to Connective Tissue Growth Factor (CTGF) and applications thereof.

### BACKGROUND OF THE INVENTION

The descriptions herein only provide background information about the present disclosure, and do not necessarily constitute prior art.

The expression of CTGF is induced by members of the Transforming Growth Factor beta (TGFβ) superfamily. This superfamily includes TGFβ-1, -2, and -3, Bone Morphogenetic Protein (BMP)-2, and activin. Many regulators (including dexamethasone, thrombin, Vascular Endothelial Growth Factor (VEGF) and angiotensin II) and environmental stress (including hyperglycemia and hypertension) also induce the expression of CTGF (see, for example, Franklin (1997) Int J Biochem Cell Biol 29:79-89; Wunderlich (2000) Graefes Arch Clin Exp Ophthalmol 238: 910-915; Denton and Abraham (2001) Curr Opin Rheumatol 13: 505-511; and Riewald (2001) Blood 97: 3109-3116; Riser et al. (2000) J Am SocNephrol 11: 25-38; and International Publication WO 00/13706).

The stimulating effect of TGFβon the expression of CTGF is rapid and long-term, and it is not necessary to persistently apply TGFβ (Igarashi et al. (1993) Mol BiolCell 4:637-645). TGFβ activates the transcription through the DNA regulatory elements present in the CTGF promoter, resulting in the enhanced expression of CTGF (Grotendorst et al. (1996) Cell Growth Differ 7: 469-480; Grotendorst and Bradham, U.S. Pat. No. 6,069,006; Holmes et al. (2001) J Biol Chem 276: 10594-10601).

The expression of CTGF is upregulated in glomerulonephritis, IgA nephropathy, interfocal and segmental glomerulosclerosis, and diabetic nephropathy (see, for example, Riser et al. (2000) J Am Soc Nephrol 11:25 -38). An increase in the number of cells expressing CTGF has also been observed in chronic tubulointerstitial injury sites, and the level of CTGF is correlated with the degree of injury (Ito et al. (1998) Kidney Int 53:853-861). In addition, in various nephropathy associated with renal parenchymal scarring and sclerosis, the expression of CTGF in glomeruli and tubulointerstitium is also increased. The elevated levels of CTGF are also associated with liver fibrosis, myocardial infarction and pulmonary fibrosis. For example, CTGF is strongly upregulated in cells from biopsies and bronchoalveolar lavage fluid in patients with spontaneous pulmonary fibrosis (IPF) (Ujike et al. (2000) Biochem Biophys Res Commun 277:448-454; Abou -Shady et al. (2000) Liver 20: 296-304; Williams et al. (2000) J Hepatol 32: 754-761). Therefore, CTGF represents an effective therapeutic target in the diseases described above.

However, no effective CTGF-targeting antibody agent is currently used in clinical practice, and there is still a need to develop novel safe and effective CTGF antibody agents.

### SUMMARY OF THE INVENTION

The present disclosure provides an anti-CTGF antibody. The anti-CTGF antibody includes anti-human CTGF full-length antibodies and antigen-binding fragments thereof

In some embodiments, the anti-CTGF antibody comprises a heavy chain variable region and a light chain variable region, wherein:

i) the heavy chain variable region comprises the same HCDR1, HCDR2 and HCDR3 sequences as those of the heavy chain variable region shown in SEQ ID NO: 6, and the light chain variable region comprises the same LCDR1, LCDR2 and LCDR3 sequences as those of the light chain variable region shown in SEQ ID NO: 7; or

ii) the heavy chain variable region comprises the same HCDR1, HCDR2 and HCDR3 sequences as those of the heavy chain variable region shown in SEQ ID NO: 8, and the light chain variable region comprises the same LCDR1, LCDR2 and LCDR3 sequences as those of the light chain variable region shown in SEQ ID NO: 9.

In some embodiments, the anti-CTGF antibody comprises a heavy chain variable region and a light chain variable region, wherein:

ii-i) the heavy chain variable region comprises the same HCDR1, HCDR2 and HCDR3 sequences as those of the heavy chain variable region shown in SEQ ID NO: 69, and the light chain variable region comprises the same LCDR1, LCDR2 and LCDR3 sequences as those of the light chain variable region shown in SEQ ID NO: 70; or

ii-ii) the heavy chain variable region comprises the same HCDR1, HCDR2 and HCDR3 sequences as those of the heavy chain variable region shown in SEQ ID NO: 85, and the light chain variable region comprises the same LCDR1, LCDR2 and LCDR3 sequences as those of the light chain variable region shown in SEQ ID NO: 70.

In some embodiments, the anti-CTGF antibody comprises a heavy chain variable region and a light chain variable region, wherein:

iii) the heavy chain variable region comprises HCDR1, HCDR2 and HCDR3 as shown in SEQ ID NO: 10, SEQ ID NO: 11 and SEQ ID NO: 12, respectively, and the light chain variable region comprises LCDR1, LCDR2 and LCDR3 as shown in SEQ ID NO: 13, SEQ ID NO: 14 and SEQ ID NO: 15, respectively; or

iv) the heavy chain variable region comprises HCDR1, HCDR2 and HCDR3 as shown in SEQ ID NO: 16, SEQ ID NO: 17 and SEQ ID NO: 18, respectively, and the light chain variable region comprises LCDR1, LCDR2 and LCDR3 as shown in SEQ ID NO: 19, SEQ ID NO: 20 and SEQ ID NO: 21, respectively.

In some embodiments, the anti-CTGF antibody comprises a heavy chain variable region and a light chain variable region, wherein:

iv-i) the heavy chain variable region comprises HCDR1, HCDR2 and HCDR3 as shown in SEQ ID NO: 71, SEQ ID NO: 72 and SEQ ID NO: 73, respectively, and the light chain variable region comprises LCDR1, LCDR2 and LCDR3 as shown in SEQ ID NO: 74, SEQ ID NO: 75 and SEQ ID NO: 76, respectively; or

iv-ii) the heavy chain variable region comprises HCDR1, HCDR2 and HCDR3 as shown in SEQ ID NO: 102, SEQ ID NO: 103 and SEQ ID NO: 104, respectively, and the light chain variable region comprises LCDR1, LCDR2 and LCDR3 as shown in SEQ ID NO: 74, SEQ ID NO: 75 and SEQ ID NO: 76, respectively.

In some embodiments, the anti-CTGF antibody is a murine antibody, a chimeric antibody, or a humanized antibody.

In some embodiments, the anti-CTGF antibody comprises a heavy chain variable region and a light chain variable region, wherein:

(v-1) the amino acid sequence of the heavy chain variable region has at least 90% sequence identity to SEQ ID NO: 6, 27, 28 or 29; and the amino acid sequence of the light chain variable region has at least 90% sequence identity to SEQ ID NO: 7, 22, 23, 24, 25, or 26;

(vi-1) the amino acid sequence of the heavy chain variable region has at least 90% sequence identity to SEQ ID NO: 8, 33, 34, 35 or 36; and the amino acid sequence of the light chain variable region has at least 90% sequence identity to SEQ ID NO: 9, 30, 31 or 32; or

(vi-i) the amino acid sequence of the heavy chain variable region has at least 90% sequence identity to SEQ ID NO: 69, 81, 82, 83, 84 or 85, and the amino acid sequence of the light chain variable region has at least 90% sequence identity to SEQ ID NO: 70, 77, 78, 79, or 80.

In some embodiments, the anti-CTGF antibody comprises a heavy chain variable region and a light chain variable region, wherein:

(v) the heavy chain variable region has at least 90%, 91%, 92%, 93%, 94%, 95%, 96%, 97%, 98%, 99% or 100% identity to the heavy chain variable region as shown in SEQ ID NO: 6, 27, 28 or 29, and the light chain variable region has at least 90%, 91%, 92%, 93%, 94%, 95%, 96%, 97%, 98%, 99% or 100% identity to the light chain variable region as shown in SEQ ID NO: 7, 22, 23, 24, 25 or 26; or

(vi) the heavy chain variable region has at least 90%, 91%, 92%, 93%, 94%, 95%, 96%, 97%, 98%, 99% or 100% identity to the heavy chain variable region as shown in SEQ ID NO: 8, 33, 34, 35 or 36, and the light chain variable region has at least 90%, 91%, 92%, 93%, 94%, 95%, 96%, 97%, 98%, 99% or 100% identity to the light chain variable region as shown in SEQ ID NO: 9, 30, 31 or 32; or

the heavy chain variable region has at least 90%, 91%, 92%, 93%, 94%, 95%, 96%, 97%, 98%, 99% or 100% identity to the heavy chain variable region as shown in SEQ ID NO: 6, and the light chain variable region has at least 90%, 91%, 92%, 93%, 94%, 95%, 96%, 97%, 98%, 99% or 100% identity to the light chain variable region as shown in SEQ ID NO: 7;

the heavy chain variable region has at least 90%, 91%, 92%, 93%, 94%, 95%, 96%, 97%, 98%, 99% or 100% identity to the heavy chain variable region as shown in SEQ ID NO: 27, 28 or 29, and the light chain variable region has at least 90%, 91%, 92%, 93%, 94%, 95%, 96%, 97%, 98%, 99% or 100% identity to the light chain variable region as shown in SEQ ID NO: 22, 23, 24, 25 or 26;

the heavy chain variable region has at least 90%, 91%, 92%, 93%, 94%, 95%, 96%, 97%, 98%, 99% or 100% identity to the heavy chain variable region as shown in SEQ ID NO: 8, and the light chain variable region has at least 90%, 91%, 92%, 93%, 94%, 95%, 96%, 97%, 98%, 99% or 100% identity to the light chain variable region as shown in SEQ ID NO: 9;

the heavy chain variable region has at least 90%, 91%, 92%, 93%, 94%, 95%, 96%, 97%, 98%, 99% or 100% identity to the heavy chain variable region as shown in SEQ ID NO: 33, 34, 35 or 36, and the light chain variable region has at least 90%, 91%, 92%, 93%, 94%, 95%, 96%, 97%, 98%, 99% or 100% identity to the light chain variable region as shown in SEQ ID NO: 30, 31 or 32.

In some embodiments, the anti-CTGF antibody comprises a heavy chain variable region and a light chain variable region, wherein:

(vi-ii) the heavy chain variable region has at least 90%, 91%, 92%, 93%, 94%, 95%, 96%, 97%, 98%, 99% or 100% identity to the heavy chain variable region as shown in SEQ ID NO: 69, 81, 82, 83, 84, or 85, and the light chain variable region has at least 90%, 91%, 92%, 93%, 94%, 95%, 96%, 97%, 98%, 99% or 100% identity to the light chain variable region as shown in SEQ ID NO: 70, 77, 78, 79 or 80.

In some embodiments of the anti-CTGF antibody, wherein the anti-CTGF antibody is a humanized antibody, which comprises a framework region of a human antibody or a framework region variant thereof, and the framework region variant has up to 10 back mutation(s) on each of the human antibody light chain framework region and/or the heavy chain framework region;

In some embodiments, the anti-CTGF antibody comprises a heavy chain variable region and a light chain variable region as described below:

(a) the light chain variable region comprises LCDR1, LCDR2 and LCDR3 as shown in SEQ ID NO: 13, SEQ ID NO: 14 and SEQ ID NO: 15, respectively, and comprises one or more amino acid back mutation(s) selected from the group consisting of 4L, 36F, 43S, 45K, 47W, 58V or 71Y, and/or the heavy chain variable region comprises HCDR1, HCDR2 and HCDR3 as shown in SEQ ID NO: 10, SEQ ID NO: 11 and SEQ ID NO: 12, respectively, and comprises one or more amino acid back mutation(s) selected from the group consisting of 28S, 30N, 49A, 75E, 76S, 93V, 94E or 104D; or

(b) the light chain variable region comprises LCDR1, LCDR2 and LCDR3 as shown in SEQ ID NO: 19, SEQ ID NO: 20 and SEQ ID NO: 21, respectively, and comprises one or more back mutation(s) selected from the group consisting of 36V, 44F, 46G or 49G, and/or the heavy chain variable region comprises HCDR1, HCDR2 and HCDR3 as shown in SEQ ID NO: 16, SEQ ID NO: 17 and SEQ ID NO: 18, respectively, and comprises one or more back mutation(s) selected from the group consisting of 44G, 49G, 27F, 48L, 67L, 71K, 78V or 80F.

In some embodiments, the anti-CTGF antibody comprises a heavy chain variable region and a light chain variable region as described below:

(b-i) the light chain variable region comprises LCDR1, LCDR2 and LCDR3 as shown in SEQ ID NO: 74, SEQ ID NO: 75 and SEQ ID NO: 76, respectively, and one or more back mutation(s) selected from the group consisting of 45P, 46W, 48Y, 69S or 70Y, and the heavy chain variable regions comprises HCDR1, HCDR2 and HCDR3 as shown in SEQ ID NO: 71, SEQ ID NO: 72 and SEQ ID NO: 73, respectively, and one or more back mutation(s) selected from the group consisting of 27F, 38K, 481, 67K, 68A, 70L or 72F; or

(b-ii) the light chain variable region comprises LCDR1, LCDR2 and LCDR3 as shown in SEQ ID NO: 74, SEQ ID NO: 75 and SEQ ID NO: 76, respectively, and comprises one or more back mutation(s) selected from the group consisting of 45P, 46W, 48Y, 69S or 70Y, and the heavy chain variable regions comprises HCDR1, HCDR2 and HCDR3 as shown in SEQ ID NO: 102, SEQ ID NO: 103 and SEQ ID NO: 104, respectively, and comprises one or more back mutation(s) selected from the group consisting of 27F, 38K, 481, 67K, 68A, 70L or 72F.

In some embodiments, the anti-CTGF antibody comprises a heavy chain variable region and a light chain variable region as described below:

(vii) the sequence of the heavy chain variable region is as shown in SEQ ID NO: 6, and the sequence of the light chain variable region is as shown in SEQ ID NO: 7;

(viii) the sequence of the heavy chain variable region is as shown in SEQ ID NO: 27, 28 or 29, and the sequence of the light chain variable region is as shown in SEQ ID NO: 22, 23, 24, 25 or 26;

(ix) the sequence of the heavy chain variable region is as shown in SEQ ID NO: 8, and the sequence of the light chain variable region is as shown in SEQ ID NO: 9; or

(x) the sequence of the heavy chain variable region is as shown in SEQ ID NO: 33, 34, 35 or 36, and the sequence of the light chain variable region is as shown in SEQ ID NO: 30, 31 or 32.

In some embodiments, the anti-CTGF antibody comprises a heavy chain variable region and a light chain variable region as described below:

(xi) the amino acid sequence of the heavy chain variable region is as shown in SEQ ID NO: 69, and the amino acid sequence of the light chain variable region is as shown in SEQ ID NO: 70; or

(xii) the amino acid sequence of the heavy chain variable region is as shown in SEQ ID NO: 81, 82, 83, 84, or 85, and the amino acid sequence of the light chain variable region is as shown in SEQ ID NO: 77, 78, 79 or 80.

In some embodiments, the anti-CTGF antibody comprises a heavy chain variable region and a light chain variable region as shown in Table a, Table b, and Table c below:

TABLE a
Combinations of the humanized antibody light chain variable
region and heavy chain variable region derived from mab147

| | Hu147-VL1 | Hu147-VL2 | Hu147-VL3 | Hu147-VL4 | Hu147-VL5 |
|---|---|---|---|---|---|
| | SEQ ID | SEQ ID | SEQ ID | SEQ ID | SEQ ID |
| | NO: 22 | NO: 23 | NO: 24 | NO: 25 | NO: 26 |
| Hu147-VH1 | Hu147-11 | Hu147-12 | Hu147-13 | Hu147-14 | Hu147-15 |
| SEQ ID | | | | | |
| NO: 27 | | | | | |
| Hu147-VH2 | Hu147-21 | Hu147-22 | Hu147-23 | Hu147-24 | Hu147-25 |
| SEQ ID | | | | | |
| NO: 28 | | | | | |
| Hu147-VH3 | Hu147-31 | Hu147-32 | Hu147-33 | Hu147-34 | Hu147-35 |
| SEQ ID | | | | | |
| NO: 29 | | | | | |

TABLE b
Combinations of mab164 humanized antibody light chain
variable region and heavy chain variable region

| | Hu164-VH5 | Hu164-VH6 | Hu164-VH7 | Hu164-VH8 |
|---|---|---|---|---|
| | SEQ ID | SEQ ID | SEQ ID | SEQ ID |
| | NO: 33 | NO: 34 | NO: 35 | NO: 36 |
| Hu164-VL7 | Hu164-57 | Hu164-67 | Hu164-77 | Hu164-87 |
| SEQ ID | | | | |
| NO: 30 | | | | |
| Hu164-VL8 | Hu164-58 | Hu164-68 | Hu164-78 | Hu164-88 |
| SEQ ID | | | | |
| NO: 31 | | | | |
| Hu164-VL9 | Hu164-59 | Hu164-69 | Hu164-79 | Hu164-89 |
| SEQ ID | | | | |
| NO: 32 | | | | |

TABLE c
Combinations of mab95 humanized antibody light chain
variable region and heavy chain variable region

| | Hu95-VH1 | Hu95-VH2 | Hu95-VH3 | Hu95-VH4 | Hu95-VH5 |
|---|---|---|---|---|---|
| | SEQ ID | SEQ ID | SEQ ID | SEQ ID | SEQ ID |
| | NO: 81 | NO: 82 | NO: 83 | NO: 84 | NO: 85 |
| Hu95-VL1 | Hu95-11 | Hu95-21 | Hu95-31 | Hu95-41 | Hu95-51 |
| SEQ ID | | | | | |
| NO: 77 | | | | | |
| Hu95-VL2 | Hu95-12 | Hu95-22 | Hu95-32 | Hu95-42 | Hu95-52 |
| SEQ ID | | | | | |
| NO: 78 | | | | | |
| Hu95-VL3 | Hu95-13 | Hu95-23 | Hu95-33 | Hu95-43 | Hu95-53 |
| SEQ ID | | | | | |
| NO: 79 | | | | | |
| Hu95-VL4 | Hu95-14 | Hu95-24 | Hu95-34 | Hu95-44 | Hu95-54 |
| SEQ ID | | | | | |
| NO: 80 | | | | | |

In some embodiments, the anti-CTGF antibody comprises a heavy chain variable region and a light chain variable region as described below:

(xiii) the amino acid sequence of the heavy chain variable region is as shown in SEQ ID NO: 27, and the amino acid sequence of the light chain variable region is as shown in SEQ ID NO: 22;

(xiv) the amino acid sequence of the heavy chain variable region is as shown in SEQ ID NO: 34, and the amino acid sequence of the light chain variable region is as shown in SEQ ID NO: 30; or

(xv) the amino acid sequence of the heavy chain variable region is as shown in SEQ ID NO: 85, and the amino acid sequence of the light chain variable region is as shown in SEQ ID NO: 77.

In some embodiments of the anti-CTGF antibody, wherein the antibody further comprises an antibody heavy chain constant region and a light chain constant region; preferably, the heavy chain constant region is selected from the group consisting of the constant regions of human IgG1, IgG2, IgG3 and IgG4 and conventional variants thereof, and the light chain constant region is selected from the group consisting of the constant regions of human antibody κ and λ chains and conventional variants thereof; more preferably, the antibody comprises a heavy chain constant region as shown in SEQ ID NO: 37 or 38 and a light chain constant region as shown in SEQ ID NO: 39 or 40.

In some embodiments, the anti-CTGF antibody comprises:

(c) a heavy chain having at least 85%, 90%, 91%, 92%, 93%, 94%, 95%, 96%, 97%, 98%, 99% or 100% identity to the heavy chain as shown in SEQ ID NO: 41, 43, 44, 45, 46, 47 or 48, and a light chain having at least 85%, 90%, 91%, 92%, 93%, 94%, 95%, 96%, 97%, 98%, 99% or 100% identity to the light chain as shown in SEQ ID NO: 42, 49, 50, 51, 52 or 53;

(d) a heavy chain having at least 85%, 90%, 91%, 92%, 93%, 94%, 95%, 96%, 97%, 98%, 99% or 100% identity to the heavy chain as shown in SEQ ID NO: 54, 56, 57, 58, 59, 60, 61, 62, or 63, and a light chain having at least 85%, 90%, 91%, 92%, 93%, 94%, 95%, 96%, 97%, 98%, 99% or 100% identity to the light chain as shown in SEQ ID NO: 55, 64, 65 or 66;

(e) a heavy chain as shown in SEQ ID NO: 41, 43, 44, 45, 46, 47 or 48, and a light chain as shown in SEQ ID NO: 42, 49, 50, 51, 52 or 53; or

(f) a heavy chain as shown in SEQ ID NO: 54, 56, 57, 58, 59, 60, 61, 62 or 63, and a light chain as shown in SEQ ID NO: 55, 64, 65, or 66.

In some embodiments, the anti-CTGF antibody comprises:

(g) a heavy chain having at least 85%, 90%, 91%, 92%, 93%, 94%, 95%, 96%, 97%, 98%, 99% or 100% sequence identity to SEQ ID NO: 86, 88, 89, 90, 91, 92, 93, 94, 95, 96, or 97, and a light chain having at least 85%, 90%, 91%, 92%, 93%, 94%, 95%, 96%, 97%, 98%, 99%, or 100% sequence identity to SEQ ID NO: 87, 98, 99, 100 or 101; or

(h) a heavy chain as shown in SEQ ID NO: 86, 88, 89, 90, 91, 92, 93, 94, 95, 96 or 97, and a light chain as shown in SEQ ID NO: 87, 98, 99, 100 or 101.

In some embodiments, the anti-CTGF antibody comprises:

(j) a heavy chain as shown in SEQ ID NO: 46, and a light chain as shown in SEQ ID NO: 49;

(k) a heavy chain as shown in SEQ ID NO: 61, and a light chain as shown in SEQ ID NO: 64; or

(l) a heavy chain as shown in SEQ ID NO: 97, and a light chain as shown in SEQ ID NO: 98.

In other aspects of the present disclosure, provided is an anti-CTGF antibody, which competitively with the anti-CTGF antibody or antigen-binding fragments thereof as described above, for the binding to human CTGF.

In other aspects of the present disclosure, provided is a nucleic acid molecule encoding the anti-CTGF antibody as described above.

In other aspects of the present disclosure, provided is a host cell comprising the nucleic acid molecule as described above.

In other aspects of the present disclosure, provided is a pharmaceutical composition, comprising a therapeutically effective amount or a prophylactically effective amount of the anti-CTGF antibody as described above, or the nucleic acid molecule as described above, and one or more pharmaceutically acceptable carriers, diluents, buffers or excipients.

In some specific embodiments, the therapeutically effective amount or prophylactically effective amount means that a unit dose of the composition comprises 0.1-3000 mg or 1-1000 mg of the anti-CTGF antibody as described above.

In other aspects of the present disclosure, provided is a method for the immunoassay or determination of CTGF, the method comprising a step of applying the anti-CTGF antibody as described above.

In other aspects of the present disclosure, provided is a method for the immunoassay or determination of CTGF, the method comprising a step of contacting the anti-CTGF antibody as described above with a subject or a sample thereof

In other aspects of the present disclosure, provided is a kit comprising the anti-CTGF antibody as described above.

In other aspects of the present disclosure, provided is a method for the treatment of CTGF-related diseases, the method comprising administering to a subject a therapeutically effective amount of the anti-CTGF antibody as described above, or the nucleic acid molecule as described above, or the pharmaceutical composition as described above, wherein the disease is preferably a fibrous disease (the fibrous disease is preferably spontaneous pulmonary fibrosis, diabetic nephropathy, diabetic retinopathy, osteoarthritis, scleroderma, chronic heart failure, liver cirrhosis or renal fibrosis), hypertension, diabetes, myocardial infarction, arthritis, CTGF-related cell proliferative disease, atherosclerosis, glaucoma or cancer (the cancer is preferably acute lymphoblastic leukemia, dermatofibroma, breast cancer, angiolipoma, angioleiomyoma, connective tissue-generating cancer, prostate cancer, ovarian cancer, colorectal cancer, pancreatic cancer, gastrointestinal cancer or liver cancer).

In other aspects of the present disclosure, provided is use of the anti-CTGF antibody as described above, or the nucleic acid molecule as described above, or the pharmaceutical composition as described above, in the preparation of a medicament for the treatment of CTGF-related diseases, the CTGF-related disease includes a fibrous disease (the fibrous disease is preferably spontaneous pulmonary fibrosis, diabetic nephropathy, diabetic retinopathy, osteoarthritis, scleroderma, chronic heart failure, liver cirrhosis or renal fibrosis), hypertension, diabetes, myocardial infarction, arthritis, CTGF-related cell proliferative disease, atherosclerosis, glaucoma or cancer (the cancer is preferably acute lymphoblastic leukemia, dermatofibroma, breast cancer, angiolipoma, angioleiomyoma, connective tissue-generating cancer, prostate cancer, ovarian cancer, colorectal cancer, pancreatic cancer, gastrointestinal cancer or liver cancer).

In other aspects of the present disclosure, provided is the anti-CTGF antibody as described above, or the nucleic acid molecule encoding the anti-CTGF antibody as described above, or the pharmaceutical composition as described above, for use in the treatment of CTGF-related diseases, the CTGF-related disease includes a fibrous disease (the fibrous disease is preferably spontaneous pulmonary fibrosis, diabetic nephropathy, diabetic retinopathy, osteoarthritis, scleroderma, chronic heart failure, liver cirrhosis or renal fibrosis), hypertension, diabetes, myocardial infarction, arthritis, CTGF-related cell proliferative disease, atherosclerosis, glaucoma or cancer (the cancer is preferably acute lymphoblastic leukemia, dermatofibroma, breast cancer, angiolipoma, angioleiomyoma, connective tissue-generating cancer, prostate cancer, ovarian cancer, colorectal cancer, pancreatic cancer, gastrointestinal cancer or liver cancer).

### DESCRIPTION OF THE DRAWINGS

FIG. 1: The affinity of humanized antibody derived from mab164 to human CTGF detected by ELISA.

FIG. 2: Experimental results of the humanized antibody derived from mab147 or mab164 in inhibiting the SU86.86 mouse xenograft tumor.

### DETAILED DESCRIPTION OF THE INVENTION

### Terminology

In order to make the present disclosure be more easily understood, certain technical and scientific terms are specifically defined below. Unless otherwise defined explicitly herein, all other technical and scientific terms used herein have the meaning commonly understood by those skilled in the art to which this disclosure belongs.

Three-letter codes and one-letter codes for amino acids used in the present disclosure are as described in J.biol.chem, 243, p3558 (1968).

Connective Tissue Growth Factor (CTGF) is a 36kD cysteine-rich heparin-binding secreted glycoprotein, which is originally isolated from human umbilical vein endothelial cells (see, for example, Bradham et al. (1991) J Cell Biol114: 1285-1294; Grotendorst and Bradham, U.S. Pat. No. 5,408,040). CTGF belongs to the protein CCN (CTGF, Cyr61, Nov) family (secreted glycoproteins), and the family includes serum-induced immediate early gene product Cyr61, putative oncogene Nov, ECM-related protein FISP-12, src-induced gene CEF-10, Wnt-induced secreted protein WISP-3 and antiproliferative protein HICP/rCOP (Brigstock (1999) Endocr Rev 20: 189-206; O'Brian et al. (1990) Mol Cell Biol 10: 3569-3577; Joliot et al. (1992) Mol Cell Biol 12: 10-21; Ryseck et al. (1990) Cell Growth and Diff 2: 225-233; Simmons et al. (1989) Proc Natl Acad Sci USA 86: 1178-1182; Pennica et al. (1998) Proc Natl Acad Sci USA, 95: 14717-14722; and Zhang et al. (1998) Mol Cell Biol 18: 6131-6141. The CCN protein is characterized by the conservative 38 cysteine residues. The 38 cysteine residues constitute over 10% of the total amino acid content and give rise to a modular structure with N-terminal and C-terminal domains. The modular structure of CTGF includes conservative motifs for insulin-like growth factor binding protein (IGF-BP) and von Willebrand factor (VWC) in the N-terminal domain, and thrombospondin (TSP1) and a cysteine-knot motif in the C-terminal domain.

As used herein, “antibody” refers to immunoglobulin, and a full-length antibody is a four-peptide chain structure connected together by interchain disulfide bond(s) between two identical heavy chains and two identical light chains. Immunoglobulin heavy chain constant regions exhibit different amino acid compositions and orders, hence present different antigenicity. Accordingly, immunoglobulins can be divided into five types, or named as immunoglobulin isotypes, namely IgM, IgD, IgG, IgA and IgE, and the corresponding heavy chains are μ, δ, γ, α and ε, respectively. According to its amino acid composition of hinge region as well as the number and location of heavy chain disulfide bonds, the same type of Ig can further be divided into different sub-types, for example, IgG can be divided into IgG1, IgG2, IgG3 and IgG4. Light chains can be divided into κ or λ, chain based on different constant regions. Each of the five types of Ig can have a kappa chain or a lambda chain.

About 110 amino acid sequences adjacent to the N-terminus of the antibody heavy and light chains are highly variable, known as variable regions (Fv regions); the rest of amino acid sequences close to the C-terminus are relatively stable, known as constant regions. The variable region includes 3 hypervariable regions (HVRs) and 4 framework regions (FRs) with relatively conservative sequences. The three hypervariable regions which determine the specificity of the antibody are also known as complementarity determining regions (CDRs). Each light chain variable region (VL) and each heavy chain variable region (VH) consists of three CDR regions and four FR regions, arranged from the amino terminus to carboxyl terminus in the following order: FR1, CDR1, FR2, CDR2, FR3, CDR3, and FR4. The three CDR regions of the light chain refer to LCDR1, LCDR2, and LCDR3, and the three CDR regions of the heavy chain refer to HCDR1, HCDR2, and HCDR3.

The antibodies of the present disclosure include murine antibodies, chimeric antibodies, and humanized antibodies.

As used herein, the term “murine antibody” refers to monoclonal antibodies against human CTGF prepared according to the knowledge and skills in the art. During the preparation, test subject is injected with CTGF antigen, and then a hybridoma expressing the antibody which possesses desired sequence or functional characteristics is isolated. In a preferred embodiment of the present disclosure, the murine anti-CTGF antibody or antigen-binding fragments thereof can further comprise a light chain constant region of murine kappa, lambda chain or variants thereof, or further comprise a heavy chain constant region of murine IgG1, IgG2, IgG3, or variants thereof

The term “chimeric antibody”, is an antibody by fusing the variable region of murine antibody to the constant region of human antibody, and such antibody can alleviate the murine antibody-induced immune response. To establish a chimeric antibody, first, a hybridoma secreting specific murine monoclonal antibody is established and variable region gene is cloned from the murine hybridoma. Then constant region gene is cloned from human antibody according to the need. The murine variable region gene is connected to the human constant region gene to form a chimeric gene, which can be subsequently inserted into an expression vector. Finally the chimeric antibody molecule will be expressed in eukaryotic or prokaryotic system. In a preferable embodiment of the present disclosure, the antibody light chain of the chimeric antibody further comprises a light chain constant region of human kappa, lambda chain or variants thereof. The antibody heavy chain of the CTGF chimeric antibody further comprises a heavy chain constant region of human IgG1, IgG2, IgG3, IgG4 or variants thereof, preferably comprises a heavy chain constant region of human IgG1, IgG2 or IgG4, or of IgG1, IgG2 or IgG4 variant with amino acid mutation(s) (such as L234A and/or L235A mutation, and/or S228P mutation).

The term “humanized antibody”, also known as CDR-grafted antibody, refers to an antibody generated by grafting the murine CDR sequences into human antibody variable region frameworks, i.e., an antibody produced in different types of human germline antibody framework sequences. Humanized antibody can overcome heterologous responses induced by chimeric antibody which carries a large number of murine protein components. Such framework sequences can be obtained from public DNA database covering germline antibody gene sequences or published references. For example, germline DNA sequences of human heavy and light chain variable region genes can be found in “VBase” human germline sequence database (available on www.mrccpe.com.ac.uk/vbase), as well as in Kabat, EA, et al. 1991 Sequences of Proteins of Immunological Interest, 5th Ed. To avoid a decrease in activity caused by the decreased immunogenicity, the framework sequences in human antibody variable region can be subjected to minimal reverse mutation(s) or back mutation(s) to maintain or increase the activity. The humanized antibodies of the present disclosure also comprise humanized antibodies on which CDR affinity maturation is performed by yeast display.

Due to the residues contacted with an antigen, the grafting of CDR can result in a decreased affinity of an antibody or antigen binding fragment thereof to the antigen due to the framework residues contacted with the antigen. Such interactions can be resulted from highly somatic mutations. Therefore, it can still be necessary to graft such donor framework amino acids onto the humanized antibody frameworks. The amino acid residues involved in antigen binding and derived from non-human antibody or antigen binding fragment thereof can be identified by checking the sequence and structure of animal monoclonal antibody variable region. The donor CDR framework amino acid residues which are different from the germ lines can be considered as being related. If it is not possible to determine the most closely related germ line, the sequence can be compared to the common sequence shared by subtypes or the animal antibody sequence with high similarity percentage. Rare framework residues are thought to be the result of a high mutation in somatic cells, and play an important role in binding.

In an embodiment of the present disclosure, the antibody or antigen binding fragment thereof further comprises a light chain constant region derived from human or murine κ, λ chain or variant thereof, or further comprises a heave chain constant region derived from human or murine IgG1, IgG2, IgG3, IgG4 or variant thereof; it can include a heavy chain constant region derived from human IgG1, IgG2 or IgG4, or from IgG1, IgG2 or IgG4 variant with amino acid mutation(s) (such as L234A and/or L235A mutation, and/or S228P mutation).

As used herein, the “conventional variants” of the human antibody heavy chain constant region and the human antibody light chain constant region refer to the human heavy or chain constant region variants disclosed in the prior art which do not change the structure and function of the antibody variable regions. Exemplary variants include IgG1, IgG2, IgG3 or IgG4 heavy chain constant region variants by site-directed modification and amino acid substitutions on the heavy chain constant region. The specific substitutions are, for example, YTE mutation, L234A and/or L235A mutations, S228P mutation, or mutations resulting in a knob-into-hole structure (making the antibody heavy chain have a combination of knob-Fc and hole-Fc), etc. These mutations have been proven to make the antibody have new properties, without changing the function of the antibody variable region.

“Human antibody (HuMAb)”, “antibody derived from human”, “fully human antibody” and “completely human antibody” can be used interchangeably, and can be antibodies derived from human or antibodies obtained from a genetically modified organism which has been “engineered” by any method known in the art to produce specific human antibodies in response to antigen stimulation, and can be produced. In some technologies, elements of human heavy and light chain loci are introduced into cell lines of organisms derived from embryonic stem cell lines, and the endogenous heavy and light chain loci in these cell lines are targeted and disrupted. The targeted endogenous heavy and light chain loci included in these cell lines are disrupted. Transgenic organisms can synthesize human antibodies specific for human antigens, and the organisms can be used to produce hybridoma that secretes human antibodies. A human antibody can also be such antibody in which the heavy and light chains are encoded by nucleotide sequences derived from one or more human DNA sources. Fully human antibodies can also be constructed by gene or chromosome transfection methods and phage display technology, or constructed from B cells activated in vitro, all of which are known in the art.

The terms “full-length antibody”, “full antibody”, “whole antibody” and “complete antibody” are used interchangeably herein and refer to an antibody in a substantially intact form, as distinguished from antigen-binding fragments defined below. The term specifically refers to antibodies that contain constant regions in the light and heavy chains.

The “antibody” of the present disclosure includes “full-length antibodies” and antigen-binding fragments thereof.

In some embodiments, the full-length antibody of the present disclosure includes full-length antibodies formed by linking the light chain variable region to the light chain constant region, and linking the heavy chain variable region to the heavy chain constant region, as shown in the combination of light and heavy chain listed in the Tables 1, 2 and 3 below. Those skilled in the art can select the light chain constant region and heavy chain constant region from various antibody sources according to actual needs, such as human antibody-derived light chain constant region and heavy chain constant region. At the same time, the various combinations of the light and heavy chain variable region described in Tables 1, 2 and 3 can form a single chain antibody (scFv), Fab or other forms of antigen-binding fragment comprising scFv or Fab.

TABLE 1
Combinations of the humanized antibody light chain variable
region and heavy chain variable region derived from mab147

| | Hu147-VL1 | Hu147-VL2 | Hu147-VL3 | Hu147-VL4 | Hu147-VL5 |
|---|---|---|---|---|---|
| Hu147-VH1 | Hu147-11 | Hu147-12 | Hu147-13 | Hu147-14 | Hu147-15 |
| Hu147-VH2 | Hu147-21 | Hu147-22 | Hu147-23 | Hu147-24 | Hu147-25 |
| Hu147-VH3 | Hu147-31 | Hu147-32 | Hu147-33 | Hu147-34 | Hu147-35 |

TABLE 2
Combinations of the light chain variable region and heavy
chain variable region of mab164 humanized antibody

| | Hu164-VH5 | Hu164-VH6 | Hu164-VH7 | Hu164-VH8 |
|---|---|---|---|---|
| Hu164-VL7 | Hu164-57 | Hu164-67 | Hu164-77 | Hu164-87 |
| Hu164-VL8 | Hu164-58 | Hu164-68 | Hu164-78 | Hu164-88 |
| Hu164-VL9 | Hu164-59 | Hu164-69 | Hu164-79 | Hu164-89 |

TABLE 3
Combinations of the light chain variable region and heavy
chain variable region of mab95 humanized antibody

| | Hu95-VH1 | Hu95-VH2 | Hu95-VH3 | Hu95-VH4 | Hu95-VH5 |
|---|---|---|---|---|---|
| Hu95-VL1 | Hu95-11 | Hu95-21 | Hu95-31 | Hu95-41 | Hu95-51 |
| Hu95-VL2 | Hu95-12 | Hu95-22 | Hu95-32 | Hu95-42 | Hu95-52 |
| Hu95-VL3 | Hu95-13 | Hu95-23 | Hu95-33 | Hu95-43 | Hu95-53 |
| Hu95-VL4 | Hu95-14 | Hu95-24 | Hu95-34 | Hu95-44 | Hu95-54 |

Note:
Hu164-57 means that the humanized antibody Hu164-57 derived from mab164 has a heavy chain variable region as described in Hu164-VH5 and a light chain variable region as described in Hu164-VL7; the others can be interpreted in the same manner.

The term “antigen-binding fragment” or “functional fragment” refers to one or more fragments of the antibody that retain the ability to specifically bind to an antigen (e.g., CTGF). It has been shown that fragments of a full-length antibody can be used to achieve function of binding to a specific antigen. Examples of the binding fragments involved in the term “antigen-binding fragment” of an antibody include (i) Fab fragment, a monovalent fragment composed of VL, VH, CL and CH1 domains; (ii) F(ab′)₂ fragment, a bivalent fragment comprising two Fab fragments connected by disulfide bridge(s) in the hinge region, (iii) Fd fragment composed of VH and CH1 domains; (iv) Fv fragment composed of the VH and VL domains of one arm of the antibody; (v) dsFv, a stable antigen-binding fragment formed by VH and VL via interchain disulfide bond(s); and (vi) diabody, bispecific antibody and multispecific antibody containing fragments such as scFv, dsFv, and Fab. In addition, the two domains, VL and VH domain, of the Fv fragment are encoded by two separate genes, however, they can be linked by a synthetic linker by using recombinant methods, to generate a single protein chain in which a monovalent molecular is formed by pairing the VL and VH domain (referred to as single chain Fv (scFv); see, e.g., Bird et al. (1988) Science 242: 423-426; and Huston et al (1988) Proc. Natl. Acad. Sci USA85:5879-5883). Such single chain antibodies are also included in the term of “antigen binding fragment” of an antibody. Such fragments of antibodies are obtained using conventional techniques known in the field, and are screened for functional fragments by using the same method as that for an intact antibody. Antigen binding portions can be produced by recombinant DNA technology or by enzymatic or chemical disruption of an intact immunoglobulin. Antibodies can be in the form of different isotypes, e.g., IgG (e.g., IgG1, IgG2, IgG3 or IgG4 subtype), IgA1, IgA2, IgD, IgE or IgM antibody.

Fab is an antibody fragment obtained by treating an IgG antibody molecule with a papain (which cleaves the amino acid residue at position 224 of the H chain), and the antibody fragment has a molecular weight of about 50,000 and has antigen binding activity, in which about a half of the N-terminal side of H chain and the entire L chain are bound together through disulfide bond(s).

“F(ab′)₂” is an antibody fragment with a molecular weight of about 100,000 and having antigen-binding activity, and it is obtained by digesting the downstream part of the two disulfide bonds in the hinge region of IgG by pepsin. F(ab′)₂ contains two Fabs connected at the hinge region.

Fab′ is an antibody fragment having a molecular weight of about 50,000 and having antigen binding activity, which is obtained by cleaving a disulfide bond at the hinge region of the above-mentioned F(ab′)₂. The Fab′ of the present disclosure can be produced by treating the F(ab′)₂ of the present disclosure which specifically recognizes CTGF and binds to the extracellular region amino acid sequences or the three-dimensional structure thereof with a reducing agent, such as dithiothreitol.

Further, the Fab′ can be produced by inserting DNA encoding Fab′ of the antibody into a prokaryotic expression vector or eukaryotic expression vector and introducing the vector into a prokaryote or eukaryote to express the Fab′.

The term “single chain antibody”, “single chain Fv” or “scFv” refers to a molecule comprising antibody heavy chain variable domain (or region; VH) connected to antibody light chain variable domain (or region; VL) by a linker. Such scFv molecules have general structure of NH₂-VL-linker-VH-COOH or NH₂-VH-linker-VL-COOH. A suitable linker in the prior art consists of repeated GGGGS amino acid sequence or variant thereof, for example, variant with 1-4 repeats (Holliger et al. (1993), Proc. Natl. Acad. Sci. USA 90:6444-6448). Other linkers that can be used in the present disclosure are described by Alfthan et al. (1995), Protein Eng. 8:725-731, Choi et al. (2001), Eur. J. Immunol. 31:94-106, Hu et al. (1996), Cancer Res. 56:3055-3061, Kipriyanov et al. (1999), J. Mol. Biol. 293:41-56 and Roovers et al. (2001), Cancer Immunol.

Diabody is an antibody fragment wherein scFv or Fab is dimerized, and it is an antibody fragment having divalent antigen binding activity. In the bivalent antigen binding activity, the two antigens can be the same or different.

Bispecific and multispecific antibody refer to an antibody that can simultaneously bind to two or more antigens or antigenic determinants, including scFv or Fab fragments that can bind CTGF.

The diabody of the present disclosure can be produced by the following steps: obtaining cDNAs encoding VH and VL of the monoclonal antibody of the present disclosure which specifically recognizes human CTGF and binds to the extracellular region or three-dimensional structure thereof, constructing DNA encoding scFv so that the length of the linker peptide is 8 or less amino acid residues, inserting the DNA into a prokaryotic expression vector or eukaryotic expression vector, and then introducing the expression vector into a prokaryote or eukaryote to express the diabody.

dsFv is obtained by substituting one amino acid residue in each of VH and VL with a cysteine residue, and then connecting the substituted polypeptides via a disulfide bond between the two cysteine residues. The amino acid residues to be substituted with a cysteine residue can be selected based on three-dimensional structure prediction of the antibody in accordance with known methods (Protein Engineering, 7, 697 (1994)).

The full-length antibodies or antigen-binding fragments of the present disclosure can be produced by the following steps: obtaining cDNAs encoding the antibody of the present disclosure which specifically recognizes human CTGF and binds to the amino acid sequence of the extracellular region or three-dimensional structure thereof, constructing DNA encoding dsFv, inserting the DNA into a prokaryotic expression vector or eukaryotic expression vector, and then introducing the expression vector into a prokaryote or eukaryote to express the dsFv.

The term “amino acid difference” or “amino acid mutation” refers to the amino acid changes or mutations in a protein or polypeptide variant compared to the original protein or polypeptide, including 1, 2, 3 or more amino acid insertions, deletions or substitutions on the basis of the original protein or polypeptide.

The term “antibody framework” or “FR region” refers to a part of the variable domain, either VL or VH, which serves as a scaffold for the antigen binding loops (CDRs) of this variable domain. Essentially, it is a variable domain without CDRs.

The term “complementarity determining region”, “CDR” or “hypervariable region” refers to one of the six hypervariable regions present in the antibody variable domain that mainly contribute to antigen binding. Generally, there are three CDRs (HCDR1, HCDR2, HCDR3) in each heavy chain variable region, and three CDRs (LCDR1, LCDR2, LCDR3) in each light chain variable region. The amino acid sequence boundaries of CDRs can be determined by any of a variety of well-known schemes, including the “Kabat” numbering criteria (see Kabat et al. (1991), “Sequences of Proteins of Immunological Interest”, 5th edition, Public Health Service, National Institutes of Health, Bethesda, Md.), “Chothia” numbering criteria (see Al-Lazikani et al., (1997) JMB 273:927-948) and ImmunoGenTics (IMGT) numbering criteria (Lefranc MP, Immunologist, 7, 132-136 (1999); Lefranc, MP, etc., Dev. Comp. Immunol., 27, 55-77 (2003), and the like. For example, for the classical format, following the Kabat criteria, the CDR amino acid residues in the heavy chain variable domain (VH) are numbered as 31-35 (HCDR1), 50-65 (HCDR2) and 95-102 (HCDR3); and the CDR amino acid residues in the light chain variable domain (VL) are numbered as 24-34 (LCDR1), 50-56 (LCDR2), and 89-97 (LCDR3). Following the Chothia criteria, the CDR amino acid residues in VH are numbered as 26-32 (HCDR1), 52-56 (HCDR2) and 95-102 (HCDR3); and the amino acid residues in VL are numbered as 26-32 (LCDR1), 50-52 (LCDR2) and 91-96 (LCDR3). By combining both Kabat and Chothia to define CDRs, the CDRs are composed of amino acid residues 26-35 (HCDR1), 50-65 (HCDR2) and 95-102 (HCDR3) in the human VH and amino acid residues 24-34 (LCDR1), 50-56 (LCDR2) and 89-97 (LCDR3) in the human VL. Following IMGT criteria, the CDR amino acid residues in VH are roughly numbered as 26-35 (CDR1), 51-57 (CDR2) and 93-102 (CDR3), and the CDR amino acid residues in VL are roughly numbered as 27-32 (CDR1), 50-52 (CDR2) and 89-97 (CDR3). Following IMGT criteria, the CDR regions of an antibody can be determined by using IMGT/DomainGap Align Program. Unless otherwise specified, the antibody variable regions and CDR sequences involved in the embodiments of the present disclosure are applicable for the “Kabat” numbering criteria.

The term “epitope” or “antigenic determinant” refers to a site on an antigen to which an immunoglobulin or antibody specifically binds (e.g., a specific site on CTGF molecule). Epitopes typically include at least 3, 4, 5, 6, 7, 8, 9, 10, 11, 12, 13, 14 or 15 contiguous or non-contiguous amino acids in a unique tertiary conformation. See, for example, Epitope Mapping Protocols in Methods in Molecular Biology, Vol. 66, G.E. Morris, Ed. (1996).

The term “specifically bind to”, “selectively bind to”, “selectively binds to” or “specifically binds to” refers to the binding of an antibody to a predetermined epitope on an antigen. Typically, the antibody binds with an affinity (KD) of less than about 10⁻⁸ M, for example, less than about 10⁻⁹ M, 10⁻¹⁰ M, 10⁻¹¹ M, 10⁻¹² M or even less.

The term “KD” refers to the dissociation equilibrium constant for particular antibody-antigen interaction. Generally, the antibody of the present disclosure binds to CTGF with a dissociation equilibrium constant (KD) of less than about 10⁻⁷ M, for example, less than about 10⁻⁸ M or 10 ⁻⁹ M, for example, the affinity of the antibody in the present disclosure to the cell surface antigen is determined by measuring KD value with FACS method.

When the term “competition” is used in the context of antigen binding proteins (e.g., neutralizing antigen binding proteins or neutralizing antibodies) that compete for the same epitope, it means that competition occurs between the antigen binding proteins, which is determined by the assays in which an antigen binding protein to be tested (e.g., an antibody or immunologically functional fragment thereof) prevents or inhibits (e.g., reduces) the specific binding of a reference antigen binding protein (e.g., a ligand or reference antibody) to a common antigen (e.g., a CTGF antigen or fragments thereof). Numerous types of competitive binding assays are available to determine whether an antigen binding protein competes with another. These assays are, for example, solid phase direct or indirect radioimmunoassay (MA), solid phase direct or indirect enzyme immunoassay (EIA), Sandwich competition assay (see, e.g., Stahli et al., 1983, Methods in Enzymology 9: 242-253); solid phase direct biotin-avidin EIA (see, e.g., Kirkland et al., 1986, J. Immunol. 137: 3614-3619), solid phase direct labeling assay, solid phase direct labeling sandwich assay (see, e.g., Harlow and Lane, 1988, Antibodies, A Laboratory Manual, Cold Spring Harbor Press); solid phase direct labeling MA with I-125 label (see, e.g., Morel et al., 1988, Molec. Immunol. 25: 7-15); solid phase direct biotin-avidin EIA (see, e.g., Cheung, et al., 1990, Virology 176: 546-552); and direct labeling MA (Moldenhauer et al., 1990, Scand. J. Immunol. 32: 77-82). Typically, the assay involves the use of a purified antigen capable of binding to a solid surface or cell loaded with both an unlabeled test antigen binding protein and a labeled reference antigen binding protein. Competitive inhibition is determined by measuring the amount of label bound to the solid surface or to the cell in the presence of the test antigen binding protein. Usually, the test antigen binding protein is present in excess. Antigen binding proteins identified by competitive assay (competing with the antigen binding protein) includes: antigen binding proteins that bind to the same epitope as the reference antigen binding protein; and antigen binding proteins that bind to an epitope that is sufficiently close to the epitope to which the reference antigen binding protein binds, where the two epitopes spatially interfere with each other to hinder the binding. Additional details regarding methods for determining competitive binding are provided in the Examples herein. Typically, when a competitive antigen binding protein is present in excess, it will inhibit (e.g., reduce) the specific binding of the reference antigen binding protein to the common antigen by at least 40-45%, 45-50%, 50-55%, 55-60%, 60-65%, 65-70%, 70-75% or 75% or even more. In some cases, the binding is inhibited by at least 80-85%, 85-90%, 90-95%, 95-97%, or 97% or even more.

As used herein, the term “nucleic acid molecule” refers to DNA molecules and RNA molecules. The nucleic acid molecule can be single-stranded or double-stranded, and is preferably double-stranded DNA or single-stranded mRNA or modified mRNA. A nucleic acid is “operably linked” when it is placed into a functional relationship with another nucleic acid sequence. For instance, a promoter or enhancer is operably linked to a coding sequence if it affects the transcription of the sequence.

Amino acid sequence “identity” refers to the percentage of the amino acid residues that are identical between the first and the second sequence when the amino acid sequences are aligned (introducing gaps when necessary) to achieve the maximum percentage of sequence identity, and any conservative substitution is not considered as part of the sequence identity. For the purpose of determining the percentage of amino acid sequence identity, the alignment can be achieved in a variety of ways within the scope of the art, for example, using publicly available computer software, such as BLAST, BLAST-2, ALIGN, ALIGN-2 or Megalign (DNASTAR) software. Those skilled in the art can determine the parameters suitable for measuring the alignment, including any algorithm required to achieve the optimum alignment over the entire length of the sequences being compared.

The term “expression vector” refers to a nucleic acid molecule capable of transporting another nucleic acid to which it has been linked. In one embodiment, the vector is a “plasmid,” which refers to a circular double stranded DNA loop into which additional DNA segments can be ligated. In another embodiment, the vector is a viral vector, wherein additional DNA segments can be ligated into the viral genome. The vectors disclosed herein are capable of self-replicating in the host cell into which they are introduced (e.g., bacterial vectors having a bacterial replication origin and episomal mammalian vectors), or can be integrated into the genome of a host cell upon introduction into the host cell, and thereby are replicated along with the host genome (e.g., non-episomal mammalian vectors).

Methods for producing and purifying antibodies and antigen-binding fragments are well known in the art, for example, A Laboratory Manual for Antibodies, Cold Spring Harbor Laboratory Press, Cold Spring Harbor, N.Y., chapters 5-8 and 15. For example, mice can be immunized with human CTGF or fragment thereof, and the resulting antibodies can then be renatured, purified, and sequenced for amino acid sequences by using conventional methods well known in the art. Antigen-binding fragments can also be prepared by conventional methods. The antibodies or antigen binding fragments of the present disclosure are engineered to incorporate one or more human framework regions onto the CDR regions derived from non-human antibody. Human FR germline sequences can be obtained from ImMunoGeneTics (IMGT) via their website http://imgt.cines.fr, or from The Immunoglobulin Facts Book, 2001, ISBN 012441351, by aligning against IMGT human antibody variable germline gene database using MOE software.

The term “host cell” refers to a cell into which an expression vector has been introduced. Host cells can include bacterial, microbial, plant or animal cells. Bacteria that are readily transformed include members of Enterobacteriaceae, such as Escherichia coli or Salmonella strains; Bacillaceae such as Bacillus subtilis; Pneumococcus; Streptococcus and Haemophilus influenzae. Suitable microorganisms include Saccharomyces cerevisiae and Pichia pastoris. Suitable animal host cell lines include CHO (Chinese Hamster Ovary cell line), 293 cells and NS0 cells.

The engineered antibodies or antigen-binding fragments of the present disclosure can be prepared and purified by conventional methods. For example, the cDNA sequences encoding the heavy and light chains can be cloned and recombined into a GS expression vector. The vectors expressing recombinant immunoglobulin can then be stably transfected into CHO cells. As a more recommended method well known in the art, mammalian expression systems will result in glycosylation, typically at highly conservative N-terminal sites in the Fc region. Stable clones can be obtained by expressing an antibody specifically binding to human CTGF. Positive clones can be expanded in serum-free culture medium in bioreactors for antibody production. Culture medium, into which an antibody has been secreted, can be purified by conventional techniques. For example, purification can be performed on Protein A or G Sepharose FF column that has been modified with buffer. The nonspecific binding components are washed out. The bound antibody is eluted by pH gradient and antibody fragments are detected by SDS-PAGE, and then pooled. The antibodies can be filtered and concentrated using common techniques. Soluble mixtures and multimers can be effectively removed by common techniques, such as size exclusion or ion exchange. The resulting product is needed to be frozen immediately, such as at −70° C., or lyophilized.

“Administering”, “dosing” or “treating” when applied to an animal, human, experimental subject, cell, tissue, organ, or biological fluid, refers to contacting an exogenous pharmaceutical, therapeutic, diagnostic agent, or composition with the animal, human, subject, cell, tissue, organ, or biological fluid. “Administering”, “dosing” or “treating” can refer, e.g., to therapeutic, pharmacokinetic, diagnostic, research, and experimental methods. The treatment of a cell encompasses contacting a reagent with the cell, as well as contacting a reagent with a fluid, where the fluid is in contact with the cell. “Administering”, “dosing” or “treating” also means in vitro or ex vivo treatments, e.g., of a cell, with a reagent, diagnostic, binding compound, or with another cell. “Treating”, as it applies to a human, veterinary, or research subject, refers to therapeutic treatment, prophylactic or preventative measures, research and diagnostic applications.

“Treatment” means to administer a therapeutic agent, such as a composition containing any of binding compounds of the present disclosure, internally or externally to a patient having one or more disease symptoms for which the agent has known therapeutic activity. Typically, the agent is administered in an amount effectively to alleviate one or more disease symptoms in the patient or population to be treated, to induce the regression of or inhibit the progression of such symptom(s) by any clinically measurable degree. The amount of a therapeutic agent that is effective to alleviate any particular disease symptom (also referred to as the “therapeutically effective amount”) can vary according to various factors such as the disease state, age, and body weight of the patient, and the ability of the drug to elicit a desired response in the patient. Whether a disease symptom has been alleviated can be assessed by any clinical measurement typically used by physicians or other skilled healthcare providers to assess the severity or progression status of that symptom. While an embodiment of the present disclosure (e.g., a treatment method or article of manufacture) may not be effective in alleviating the target disease symptom(s) in every patient, it should alleviate the target disease symptom(s) in a statistically significant number of patients as determined by any statistical test known in the art such as Student's t-test, chi-square test, U-test according to Mann and Whitney, Kruskal-Wallis test (H-test), Jonckheere-Terpstra-test and Wilcoxon-test.

“Conservative modification” or “conservative substitution or replacement” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity of the protein. Those skilled in the art understand that, generally, a single amino acid substitution in a non-essential region of a polypeptide does not substantially change the biological activity (see, for example, Watson et al. (1987) Molecular Biology of the Gene, The Benjamin/Cummings Pub. Co., Page 224, (4th edition)). In addition, substitutions with structurally or functionally similar amino acids are less likely to disrupt biological activity. Exemplary conservative substitutions are set forth below.

| Original residue | Conservative substitution |
|---|---|
| Ala (A) | Gly; Ser |
| Arg(R) | Lys; His |
| Asn (N) | Gln; His; Asp |
| Asp (D) | Glu; Asn |
| Cys (C) | Ser; Ala; Val |
| Gln(Q) | Asn; Glu |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro(P) | Ala |
| Ser(S) | Thr |
| Thr(T) | Ser |
| Trp (W) | Tyr; Phe |
| Tyr (Y) | Trp; Phe |
| Val (V) | Ile; Leu |

“Effective amount” or “effective dose” refers to the amount of a medicament, compound, or pharmaceutical composition necessary to obtain any one or more beneficial or desired results. For prophylactic applications, beneficial or desired results include elimination or reduction of risk, reduction of severity, or delay of the onset of the disease, including the biochemical, histological, and behavioral manifestations of the condition, its complications, and intermediate pathological phenotypes during the development of the condition. For therapeutic applications, beneficial or desired results include clinical results, such as reduction of the incidence of various conditions associated with target antigen of the present disclosure or improvement of one or more symptoms of the condition, reduction of the dosage/dose of other agents required to treat the condition, enhancement of the efficacy of another agent, and/or delay of the progression of the condition associated with the target antigen of the present disclosure in patients.

“Exogenous” refers to substances produced outside the organisms, cells, or humans according to circumstances. “Endogenous” refers to substances produced in the cells, organisms, or human bodies according to circumstances.

“Homology” refers to the sequence similarity between two polynucleotide sequences or between two polypeptide sequences. When a position in both of the two sequences to be compared is occupied by the same base or amino acid monomer subunit, e.g., if a position in each of two DNA molecules is occupied by adenine, then the molecules are homologous at that position. The percentage of homology between two sequences is a function of the number of matching or homologous positions shared by the two sequences divided by the number of positions to be compared and then multiplied by 100. For example, when two sequences are optimally aligned, if 6 out of 10 positions in the two sequences are matched or homologous, then the two sequences are 60% homologous; if 95 out of 100 positions in the two sequences are matched or homologous, then the two sequences are 95% homologous. Generally, when two sequences are aligned, comparison is performed to give the maximum homology percentage. For example, the comparison can be performed by the BLAST algorithm, in which the parameters of the algorithm are selected to give the maximum match between each sequence over the entire length of each reference sequence. The following references refer to the BLAST algorithm frequently used for sequence analysis: BLAST algorithm (BLAST ALGORITHMS): Altschul, SF et al., (1990) J. Mol. Biol. 215:403-410; Gish, W. et al., (1993) Nature Genet. 3:266-272; Madden, TL et al., (1996) Meth. Enzymol. 266:131-141; Altschul, SF et al., (1997) Nucleic Acids Res. 25:3389-3402; Zhang, J. et al. (1997) Genome Res. 7:649-656. Other conventional BLAST algorithms such as those available from NCBI BLAST are also well known to those skilled in the art.

As used herein, the expressions “cell,” “cell line,” and “cell culture” are used interchangeably and all such designations include progeny. Thus, the words “transformant” and “transformed cell” include the primary subject cells and cultures derived therefrom regardless of the number of passages. It should be also understood that all progeny cannot be precisely identical in DNA content, due to intentional or unintentional mutations. Mutant progeny that have the same function or biological activity as that screened in the originally transformed cells are included. Where distinct designations are intended to, it will be clearly understood from the context.

As used herein, “polymerase chain reaction” or “PCR” refers to a procedure or technique in which minute amounts of a specific portion of nucleic acid, RNA and/or DNA, are amplified as described in, e.g., U.S. Pat. No. 4,683,195. Generally, sequence information at the ends of or beyond the region of interest needs to be available, such that oligonucleotide primers can be designed; these primers will be identical or similar in sequence to opposite strands of the template to be amplified. The 5′ terminal nucleotides of the two primers can be consistent with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al. (1987) Cold Spring Harbor Symp. Ouant. Biol. 51:263; Erlich editors, (1989) PCR TECHNOLOGY (Stockton Press, N.Y.). The PCR used in the present disclosure is considered to be one, but not the only, example of polymerase reaction method for amplifying a test nucleic acid sample. The method comprises use of nucleic acid sequences known as primers together with nucleic acid polymerase to amplify or generate a specific portion of nucleic acid.

“Isolated” refers to a purified state, in which the designated molecule is substantially free of other biological molecules, such as nucleic acids, proteins, lipids, carbohydrates, or other materials, such as cell debris and growth medium. In general, the term “isolated” is not intended to mean the complete absence of these materials or the absence of water, buffers or salts, unless they are present in an amount that significantly interferes with the experimental or therapeutic use of the compound as described herein.

“Optional” or “optionally” means that the event or circumstance that follows may but does not necessarily occur, and the description includes the instances in which the event or circumstance does or does not occur.

“Pharmaceutical composition” refers to a mixture comprising one or more compounds according to the present disclosure or a physiologically/pharmaceutically acceptable salt or prodrug thereof and other chemical components, such as physiologically/pharmaceutically acceptable carriers and excipients. The pharmaceutical composition aims at promoting the administration to an organism, facilitating the absorption of the active ingredient and thereby exerting a biological effect.

The term “pharmaceutically acceptable carrier” refers to any inactive substance suitable for use in a formulation for the delivery of antibodies or antigen-binding fragments. A carrier can be an anti-adhesive agent, adhesive agent, coating agent, disintegrating agent, filler or diluent, preservative (such as antioxidant, antibacterial or antifungal agent), sweetener, absorption delaying agent, wetting agent, emulsifier, buffer, and the like. Examples of suitable pharmaceutically acceptable carriers include water, ethanol, polyol (such as glycerol, propylene glycol, polyethylene glycol, and the like), dextrose, vegetable oil (such as olive oil), saline, buffer, buffered saline, and isotonic agent (such as sugars, polyol, sorbitol and sodium chloride).

In addition, the present disclosure includes an agent for treating a disease associated with target antigen (such as CTGF) positive cells, the agent comprising the anti-CTGF antibody or antibody fragment thereof of the present disclosure as an active ingredient. The active ingredient is administered to the subject in a therapeutically effective amount, and is capable of treating a disease associated with CTGF-positive cells in the subject. The therapeutically effective amount mean that a unit dose of the composition comprises 0.1-3000 mg of the full-length antibody or antigen-binding fragment thereof that specifically binds to human CTGF as described above.

The CTGF-related disease of the present disclosure is not limited, as long as it is a disease related to CTGF. For example, the therapeutic response induced by the molecule of the present disclosure can play the role by binding to human CTGF, and then preventing CTGF from binding to its receptor/ligand, or via killing the tumor cells over-expressing CTGF. Therefore, the molecules of the present disclosure, when present in a preparation or formulation suitable for therapeutic applications, are very useful for such persons suffering from tumor or cancer, optionally including pulmonary fibrosis, renal fibrosis, tumor development and growth, glaucoma, cell proliferative disease, cataract, choroidal neovascularization, amotio retinae, proliferative vitreoretinopathy, macular degeneration, diabetic retinopathy, corneal scarring and corneal opacity, cyst, reduced vascular calcification, pancreatic ductal adenocarcinoma, pancreatic cancer, melanoma, radiation-induced fibrosis (RIF), idiopathic pulmonary fibrosis, pulmonary remodeling disease selected from the group consisting of asthma, chronic bronchitis, chronic obstructive pulmonary disease (COPD), cystic fibrosis, emphysema, etc., preferably pancreatic cancer, pulmonary fibrosis, and renal fibrosis.

In addition, the present disclosure relates to methods for the immunoassay or determination of target antigens (for example, CTGF), reagents for the immunoassay or determination of target antigens (for example, CTGF), methods for the immunoassay or determination of cells expressing target antigens (for example, CTGF), and the diagnostic agents for diagnosing diseases associated with target antigen (for example, CTGF)-positive cells, comprising the antibody or antibody fragment of the present disclosure (as an active ingredient) that specifically recognizes the target antigens (for example, human CTGF) and binds to the extracellular amino acid sequences or to the tertiary structure thereof.

In the present disclosure, the method for detecting or measuring the amount of the target antigen (e.g. CTGF) can be any known method. For example, it includes immunoassay or determination methods.

The immunoassay or determination method is a method of detecting or measuring the amount of antibody or antigen using a labeled antigen or antibody. Examples of the immunoassay or determination methods include radioactive substance-labeled immunoantibody method (RIA), enzyme immunoassay (EIA or ELISA), fluorescence immunoassay (FIA), luminescence immunoassay, western blotting, physicochemical method, and the like.

The above-mentioned diseases associated with CTGF-positive cells can be diagnosed by detecting or measuring the CTGF-expressing cells using the antibodies or antibody fragments of the present disclosure.

Cells expressing the polypeptide can be detected by the known immunodetection methods, preferably by immunoprecipitation, fluorescent cell staining, immunotissue staining, and the like. In addition, the method such as fluorescent antibody staining method with the FMAT8100HTS system (Applied Biosystem) can be used.

In the present disclosure, samples to be detected or measured for the target antigen (e.g. CTGF) are not particularly limited, as long as they are possible to contain cells expressing the target antigen (e.g. CTGF), such as tissue, cells, blood, plasma, serum, pancreatic juice, urine, stool, tissue fluid or culture medium.

Dependent on the required diagnostic method, the diagnostic agent comprising the monoclonal antibody or antibody fragment thereof of the present disclosure can also contain reagents for performing an antigen-antibody reaction or reagents for detecting the reaction. The reagents for performing an antigen-antibody reaction include buffers, salts and the like. The reagents used for detection include agents commonly used in immunoassay or immunodetection methods, for example, a labeled secondary antibody that recognizes the monoclonal antibody, antibody fragment or conjugate thereof, and a substrate corresponding to the label.

The details of one or more embodiments of the present disclosure are set forth in the above specification. The preferred methods and materials are described below, although any method and material similar or identical to those described herein can be used in the practice or testing of the present disclosure. Through the specification and claims, other features, purposes and advantages of the present disclosure will become apparent. In the specification and claims, the singular forms are intented to include plural forms, unless the context clearly dictates otherwise. Unless otherwise defined explicitly herein, all technical and scientific terms used herein have the meaning commonly understood by those skilled in the art to which this disclosure belongs. All patents and publications cited in the specification are incorporated by reference. The following examples are provided to more fully illustrate the preferred embodiments of the present disclosure. These examples should not be construed as limiting the scope of the present disclosure in any way, and the scope of the present disclosure is defined by the claims.

### EXAMPLES

### Example 1. Preparation of CTGF Antigen and Antibody

### I. Design and Expression of Antigen

Human CTGF protein (Genbank number: NM 001901.2) was used as a template to design the amino acid sequence of the antigen and the protein used for detection. Optionally, the CTGF protein or the fragment thereof was fused with various tags, and separately cloned into pTT5 vector or pXC vector, and transiently expressed in 293 cells or stably expressed in LONZA CHO cells to result in the antigens and proteins used for detection of the present disclosure. The following CTGFantigens refer to human CTGF, unless specifically indicated.

Human CTGF full-length protein (for immunization),
SEQ ID NO: 1
QNCSGPCRCPDEPAPRCPAGVSLVLDGCGCCRVCAKQLGELCTERDPCDPHKGLFC
DFGSPANRKIGVCTAKDGAPCIFGGTVYRSGESFQSSCKYQCTCLDGAVGCMPLCSMDVR
LPSPDCPFPRRVKLPGKCCEEWVCDEPKDQTVVGPALAAYRLEDTFGPDPTMIRANCLVQ
TTEWSACSKTCGMGISTRVTNDNASCRLEKQSRLCMVRPCEADLEENIKKGKKCIRTPKIS
KPIKFELSGCTSMKTYRAKFCGVCTDGRCCTPHRTTTLPVEFKCPDGEVMKKNMMFIKTC
ACHYNCPGDNDIFESLYYRKMYGDMA
CTGF-his (a fusion protein of human CTGF mature protein with his tag) can be
used for detection,
SEQ ID NO: 2
MEFGLSWLFLVAILKGVQCQNCSGPCRCPDEPAPRCPAGVSLVLDGCGCCRVCAKQL
GELCTERDPCDPHKGLFCDFGSPANRKIGVCTAKDGAPCIFGGTVYRSGESFQSSCKYQCTCL
DGAVGCMPLCSMDVRLPSPDCPFPRRVKLPGKCCEEWVCDEPKDQTVVGPALAAYRLEDTFG
PDPTMIRANCLVQTTEWSACSKTCGMGISTRVTNDNASCRLEKQSRLCMVRPCEADLEENIKK
GKKCIRTPKISKPIKFELSGCTSMKTYRAKFCGVCTDGRCCTPHRTTTLPVEFKCPDGEVMKK
NMMFIKTCACHYNCPGDNDIFESLYYRKMYGDMAHHHHHH
(Note: The single underline represents signal peptide, the italic represents the
extracellular region of hCTGF, and the double underline represents his tag.)
mCTGF-mFc (a fusion protein of mouse CTGF coding region with mouse Fc tag,
which can be used for detection)
SEQ ID NO: 3
MEFGLSWLFLVAILKGVQCQDCSAQCQCAAEAAPHCPAGVSLVLDGCGCCRVCAKQL
GELCTERDPCDPHKGLFCDFGSPANRKIGVCTAKDGAPCVFGGSVYRSGESFQSSCKYQCTCL
DGAVGCVPLCSMDVRLPSPDCPFPRRVKLPGKCCEEWVCDEPKDRTAVGPALAAYRLEDTFGP
DPTMMRANCLVQTTEWSACSKTCGMGISTRVTNDNTFCRLEKQSRECMVRPCEADLEENIKKG
KKCIRTPKIAKPVKFELSGCTSVKTYRAKFCGVCTDGRCCTPHRTTTLPVEFKCPDGEIMKKN
MMFIKTCACHYNCPGDNDIFESLYYRKMYGDMAEPRGPTIKPCPPCKCPAPNLLGGPSVFIFP
PKIKDVLMISLSPIVTCVVVDVSEDDPDVQISWFVNNVEVHTAQTQTHREDYNSTLRVVS
ALPIQHQDWMSGKEFKCKVNNKDLPAPIERTISKPKGSVRAPQVYVLPPPEEEMTKKQVT
LTCMVTDFMPEDIYVEWTNNGKTELNYKNTEPVLDSDGSYFMYSKLRVEKKNWVERNS
YSCSVVHEGLHNHHTTKSFSRTPGK
(Note: The single underline represents signal peptide, the italic represents the mouse
CTGF coding region, and the double underline represents mFc tag.)
Mouse CTGF protein-His tag,
SEQ ID NO: 4
MEFGLSWLFLVAILKGVQCQDCSAQCQCAAEAAPHCPAGVSLVLDGCGCCRVCAKQL
GELCTERDPCDPHKGLFCDFGSPANRKIGVCTAKDGAPCVFGGSVYRSGESFQSSCKYQCTCL
DGAVGCVPLCSMDVRLPSPDCPFPRRVKLPGKCCEEWVCDEPKDRTAVGPALAAYRLEDTFGP
DPTMMRANCLVQTTEWSACSKTCGMGISTRVTNDNTFCRLEKQSRECMVRPCEADLEENIKKG
KKCIRTPKIAKPVKFELSGCTSVKTYRAKFCGVCTDGRCCTPHRTTTLPVEFKCPDGEIMKKN
MMFIKTCACEIYNCPGDNDIFESLYYRKMYGDMAGHHHHHH
(Note: The single underline represents signal peptide, the italic represents the mouse
CTGF coding region, and the double underline represents his tag.)
Cynomolgus CTGF-Fc (a fusion protein of cynomolgus CTGF coding region with
Fc, which can be used for detection),
SEQ ID NO: 5
MEFGLSWLFLVAILKGVQCQNCSGPCRCPAEQAPRCPAGVSLVLDGCGCCRVCAKQL
GELCTERDPCDPHKGLFCDFGSPANRKIGVCTAKDGAPCIFGGTVYRSGESFQSSCKYQCTCL
DGAVGCMPLCSMDVRLPSPDCPFPRRVKLPGKCCEEWVCDEPKDQTWGPALAAYRLEDTFG
PDPTMIRANCLVQTTEWSACSKTCGMGISTRVTNDNASCRLEKQSRLCMVRPCEADLEENIKK
GKKCIRTPKISKPIKFELSGCTSVKTYRAKFCGVCTDGRCCTPHRTTTLPVEFKCPDGEVMKKN
MMFIKTCACHYNCPGDNDIFESLYYRKMYGDMAEPKSSDKTHTCPPCPAPELLGGPSVFLFPP
KPKDTLMISRTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSC
SVMHEALHNHYTQKSLSLSPGK
(Note: The single underline represents signal peptide, the italic represents the
cynomolgus CTGF coding region, and the double underline represents Fc tag.)

### II. Purification of CTGF-related Recombinant Protein, and Purification of Anti-human CTGF Hybridoma Antibody and Recombinant Antibody

### 1. Separation and Purification of Hybridoma Supernatant by Protein G Affinity Chromatography

For the purification of mouse hybridoma supernatant, affinity chromatography performed with Protein G was preferable. The resulting hybridoma after culturing was centrifuged and the supernatant was taken out, and based on the volume of the supernatant, 10-15% volume of 1 M Tris-HCl (pH 8.0-8.5) was added to adjust pH of the supernatant to neutral. The Protein G column was washed with 3-5×column volume of 6 M guanidine hydrochloride, and then washed with 3-5×column volume of pure water; the column was equilibrated with 3-5×column volume of 1×PBS (pH 7.4); the cell supernatant was loaded at a low flow rate for binding, and the flow rate was controlled so that the retention time was about 1 min or longer; the column was washed with 3-5×column volume of 1×PBS (pH 7.4) until the UV absorption dropped to the baseline; the samples were eluted with 0.1 M acetic acid/sodium acetate (pH 3.0) buffer, the elution peaks were pooled according to UV detection. The eluted product was rapidly adjusted to pH 5-6 with 1 M Tris-HCl (pH 8.0). The eluted product can be subjected to solution replacement according to methods well-known to those skilled in the art, for example, replacing the solution with a desired buffer system by ultrafiltration-concentration with an ultrafiltration tube, or replacing the solution with a desired buffer system by using molecular exclusion such as G-25 desalting, or removing the aggregates from the eluted product to improve the purity of the sample by using high-resolution molecular exclusion column such as Superdex 200.

2. Purification of Antibody with Protein A Affinity Chromatography

First, the cell culture supernatant expressing the antibody was centrifuged at a high speed to collect the supernatant. The Protein A affinity column was washed with 3-5×column volume of 6 M guanidine hydrochloride, and then washed with 3-5×column volume of pure water. The column was equilibrated with 3-5×column volume of, for example, 1×PBS (pH 7.4). The cell supernatant was loaded at a low flow rate for binding, and the flow rate was controlled so that the retention time was about 1 min or longer; once the binding was finished, the column was washed with 3-5×column volume of 1×PBS (pH 7.4) until the UV absorption dropped to the baseline The samples were eluted with 0.1 M acetic acid/sodium acetate (pH 3.0-3.5) buffer, the elution peaks were pooled according to UV detection. The eluted product was rapidly adjusted with 1 M Tris-HCl (pH 8.0) to pH 5-6. The eluted product can be subjected to solution replacement according to methods well-known to those skilled in the art, for example, replacing the solution with a desired buffer system by ultrafiltration-concentration with an ultrafiltration tube, or replacing the solution with a desired buffer system by using molecular exclusion such as G-25 desalting, or removing the aggregates from the eluted product to improve the purity of the sample by using high-resolution molecular exclusion column such as Superdex 200.

### 3. Purification of CTGF-related Recombinant Proteins (Such as Human CTGF-his) by Nickel Column

The cell expression supernatant sample was centrifuged at high speed to remove impurities, the buffer was replaced with PBS, and imidazole was added to a final concentration of 5 mM. The nickel column was equilibrated with PBS solution containing 5 mM imidazole, and washed with 2-5×column volume. The supernatant sample after replacement was loaded onto the column for binding, and nickel columns avaliable from different companies can be selected as the media. The column was washed with PBS solution containing 5 mM imidazole until the A280 reading dropped to the baseline. The chromatography column was rinsed with PBS+10 mM imidazole to remove the non-specifically bound protein impurities, and the effluent was collected. The target protein was eluted with PBS solution containing 300 mM imidazole, and the elution peak was collected.

The collected elution product was concentrated and then can be further purified by gel chromatography Superdex200 (GE) with PBS as a mobile phase, to remove aggregates and impurity protein peaks, and to collect the elution peak of the target product. The resulting protein was identified by electrophoresis, peptide mapping and LC-MS, and the correct proteins were aliquoted for use.

4. Purification of CTGF-related Recombinant Proteins (Such as Human CTGF-Fc, Cynomolgus CTGF-Fc) with Protein A Affinity Chromatography

First, the culture supernatant was centrifuged at a high speed to collect the supernatant. The Protein A affinity column was washed with 3-5×column volume of 6 M guanidine hydrochloride, and then washed with 3-5×column volume of pure water. The column was equilibrated with 3-5×column volume of, for example, 1×PBS (pH 7.4). The cell supernatant was loaded at a low flow rate for binding, and the flow rate was controlled so that the retention time was about 1 min or longer; once the binding was finished, the column was washed with 3-5×column volume of 1 ×PB S (pH 7.4) until the UV absorption dropped to the baseline The samples were eluted with 0.1 M acetic acid/sodium acetate (pH 3.0-3.5) buffer, and the elution peaks were pooled according to UV detection.

The collected elution product was concentrated and then can be further purified by gel chromatography Superdex200 (GE) with PBS as a mobile phase, to remove aggregates and impurity protein peaks, and to collect the elution peak of the target product. The resulting protein was identified by electrophoresis, peptide mapping and LC-MS, and the correct proteins were aliquoted for use.

### Example 2. Preparation of Anti-human CTGF Monoclonal Antibody

### I. Immunization

The anti-human CTGF monoclonal antibodies were produced by immunizing mice. SJL white mice, female, 6-8 weeks old were used for experiment (Beijing Charles River Experimental Animal Technology Co., Ltd., animal production license number: SOCK (Beijing) 2012-0001). Feeding environment: SPF level. After the mice were purchased, they were kept in the laboratory environment for 1 week, with a light/dark cycle of 12/12 hours, at temperature of 20-25° C.; humidity of 40-60%. The mice adapted to the environment were immunized according to the following schemes. CTGF (R&D) and mCTGF-mFc were used as antigen for immunization.

Immunization scheme: mice were immunized with CTGF protein, 25 micrograms/mouse/time, via intraperitoneal injection. 100 μl/mouse was injected intraperitoneally (IP) on day 0, and then booster immunization was performed once every 14 days. Blood samples were collected on day 21, 35, 49 and 63, the antibody titer in mouse serum was determined by ELISA method. After 7 to 9 immunizations, mice with a high serum antibody titer approaching to the plateau were selected for splenocyte fusion. Three days before the splenocyte fusion, solution of CTGF protein antigen prepared in saline was intraperitoneally injected (IP), 25 μg/mouse, for booster immunization.

### II. Splenocyte Fusion

Hybridoma cells were obtained by fusing splenic lymphocytes with myeloma Sp2/0 (ATCC® CRL-8287™) by using a PEG-mediated fusion procedure. The fused hybridoma cells were resuspended in complete medium (IMDM medium comprising 20% FBS, 1×HAT, 1×OPI) at a density of 0.5-1×10⁶/ml, seeded in a 96-well plate with 100 μl/well, incubated at 37° C. and 5% CO₂ for 3-4 days, supplemented with 100 μl/well of HAT complete medium, and continued to be cultured for 3-4 days until forming pinpointed clones. The supernatant was removed, 200 μl/well of HT complete medium (IMDM medium comprising 20% FBS, 1×HT and 1×OPI) was added, incubated at 37° C., 5% CO₂ for 3 days and then subjected to ELISA detection.

### III. Screening of Hybridoma Cells

According to the growth density of hybridoma cells, the hybridoma culture supernatant was detected by binding ELISA method. The cell supernatant in positive wells was detected by ELISA assay, for the binding with monkey CTGF/mouse CTGF/human CTGF protein (the specific procedures were as the same as those of Example 3). The cells in the wells positive for protein ELISA binding assay were timely expanded and cryopreserved; and were subcloned 2 to 3 times until a single cell clone was obtained.

Cells after each subcloning were also tested by CTGF binding ELISA. The hybridoma clones were obtained by screening via the above assay. The antibodies were further prepared by serum-free cell culture method. The antibodies were purified according to the example of purification, for use in the test examples.

### IV. Sequencing of the Positive Hybridoma Clones

The procedures of cloning the sequences from the positive hybridoma were as follows. Hybridoma cells in logarithmic growth phase were collected. RNAs were extracted with Trizol (Invitrogen, Cat No. 15596-018) according to the kit's instruction and were reversely transcribed with PrimeScript™ Reverse Transcription kit (Takara, Cat No. 2680A). The cDNAs resulting from reverse transcription were amplified by PCR using mouse Ig-Primer Set (Novagen, TB326 Rev. B 0503), and the amplified products were subjected to sequencing. After sequencing, murine anti-CTGF antibodies mab147, mab164 and mab95 were obtained. The amino acid sequences of the variable regions are as follows:

mab147 VH amino acid sequence is as shown in SEQ ID NO: 6,
EVQLVESGGGLVQPEGSLKLSCAASGFSFNTYAMNWVRQAPGKGLEWVARIRTKSNNYATYYAD
SVKDRFTISRDDSESMLYLQMNNLKTEDTAMYYCVETGFAYWDQGTLVTVSA
mab147 VL amino acid sequence is as shown in SEQ ID NO: 7,
QIVLTQSPAIMSASPGEKVTITCSASSSVSYMHWFQQKPGTSPKLWIYSTSNLASGVPARFSGSGS
GTSYSLTISRMEAEDAATYYCQQRSSYPLTFGAGTKLELK
mab164 VH amino acid sequence is as shown in SEQ ID NO: 8,
QVQLKQSGPGLVQPSQSLSITCTVSGFSLTTFGVHWIRQSPGKGLEWLGVIWRRGGTDYNAAF
MSRLSITKDNSRSHVFFKMTSLQTDDSAIYYCARDGGFDYWGQGTTLTVSS
mab164 VL amino acid sequence is as shown in SEQ ID NO: 9,
QAVVTQESALTTSPGGTVILTCRSSIGAVTTSNYANWVQEKPDHLFTGLIGGTSNRAPGVPVRFS
GSLIGDKAALTITGAQAEDDAMYFCALWYSTHYVFGGGTKVTVL
mab95 VH amino acid sequence is as shown in SEQ ID NO: 69,
EVLLQOSGPVLVKPGPSVKISCKASGFTFTNYDIHWVKQSHGKSLEWIGLVYPYNGGTAYNQKF
KDKATLTFDTSSSTAYMELNSLTSEDSAVYYCARWGLIPGTTSYFDVWGTGTTVTVSS
mab95 VL amino acid sequence is as shown in SEQ ID NO: 70,
QIVLSQSPPILSASPGEKVTMTCRASSSVSYIHWYQQKPRSSPKPWIYATSNLASGVPARFSGSGSG
TSYSLTISRVEAEDAATYYCQQWNSNPWTFGGGTRLEIK

In the above-mentioned mab147, mab164 and mab95 antibody variable region sequences, the italics represents the FR sequence, the underlined italics represents the CDR sequences, and the sequence order is FR1-CDR1-FR2-CDR2-FR3-CDR3-FR4.

TABLE 4
The amino acid sequences of CDR regions of
anti-CTGF antibody heavy chain and light chain*

| Antibody | Heavy chain | | Light chain | |
|---|---|---|---|---|
| mabl47 | HCDR1 | TYAMN | LCDR1 | SASSSVSYMH |
| | | SEQ ID NO: 10 | | SEQIDNO: 13 |
| | HCDR2 | RIRTKSNNYATYYA | LCDR2 | STSNLAS |
| | | DSVKD | | SEQ ID NO: 14 |
| | | SEQ ID NO: 11 | | |
| | HCDR3 | TGFAY | LCDR3 | QQRSSYPLT |
| | | SEQ ID NO: 12 | | SEQ ID NO: 15 |
| mabl64 | HCDR1 | TFGVH | LCDR1 | RSSIGAVTTSNYAN |
| | | SEQ ID NO: 16 | | SEQ ID NO: 19 |
| | HCDR2 | VIWRRGGTDYNAA | LCDR2 | GTSNRAP |
| | | FMS | | SEQ ID NO: 20 |
| | | SEQ ID NO: 17 | | |
| | HCDR3 | DGGFDY | LCDR3 | ALWYSTHYV |
| | | SEQ ID NO: 18 | | SEQ ID NO: 21 |
| mab95 | HCDR1 | NYDIH | LCDR1 | RASSSVSYIH |
| | | SEQ ID NO: 71 | | SEQ ID NO: 74 |
| | HCDR2 | LVYPYNGGTAYNQ | LCDR2 | ATSNLAS |
| | | KFKD | | SEQ ID NO: 75 |
| | | SEQ ID NO: 72 | | |
| | HCDR3 | WGLIPGTTSYFDV | LCDR3 | QQWNSNPWT |
| | | SEQ ID NO: 73 | | SEQ ID NO: 76 |

*The CDRs in the table are determined by the Kabat numbering criteria.

### V. Preparation of Human IgG1 Chimeric Antibody

The candidate molecules mab147, mab164 and mab95 were amplified and sequenced to obtain the gene sequences encoding the variable regions. Forward and reverse primers were designed on the basis of the sequences obtained by sequencing, the gene to be sequenced was used as a template to construct the VH/VK gene fragment for each antibody via PCR, and then inserted into the expression vector pHr (with a signal peptide and hIgG1/hkappa/hlambda constant region gene (CH1-Fc/CL) fragment) via homologous recombination to construct an expression plasmid for the full-length of recombinant chimeric antibody VH-CH1-Fc-pHr/VL-CL-pHr, resulting in three chimeric antibodies Ch147, Ch164 and Ch95.

### VI. Humanization of Murine Anti-human CTGF Antibody

The heavy chain and light chain variable region germline genes having high homology with murine antibodies were selected as templates by aligning against IMGT (http://imgt.cines.fr) human antibody heavy and light chain variable germline gene database by using MOE software (Molecular Operating Environment) software. The murine antibody CDRs were grafted into the corresponding human templates to form variable region sequences in an order of FR1-CDR1-FR2-CDR2-FR3 -CDR3 -FR4.

### 1. Humanization of Mab147

For murine antibody mab147, the light chain templates for humanization were IGKV3-11*01 and IGKJ2*01, or IGKV1-39*01 and IGKJ2*01, and the heavy chain templates for humanization were IGHV3-72*01 and IGHJ1*01. Each of the CDRs of the murine antibody mab147 were grafted into its human template, and then the amino acids of the FR of the humanized antibody were subjected to back mutation. Among them, the back mutation(s) in the light chain variable region include one or more selected from the group consisting of 4L, 36F, 43S, 45K, 47W, 58V or 71Y, and the back mutation(s) in the heavy chain variable region include one or more selected from the group consisting of 28S, 30N, 49A, 75E, 76S, 93V, 94E, or 104D (the back mutation positions in the light and heavy chain variable regions were determined according to the Kabat numbering criteria), resulting in the light chain variable regions and the heavy chain variable regions with different sequences. The humanized antibodies comprising the CDRs of mab147 were obtained, and the variable region sequences thereof are as follows:

The specific variable region sequences of the humanized antibodies of mAb147 are as follows:

The amino acid sequence of Hu147-VL1 is as shown in
SEQ ID NO: 22
EIVLTQSPATLSLSPGERATLSCSASSSVSYMHWFQQKPGQSPRLLIYSTSNLASGIPARFSG
SGSGTDYTLTISSLEPEDFAVYYCQQRSSYPLTFGQGTKLEIK
The amino acid sequence of Hu147-VL2 is as shown in
SEQ ID NO: 23
EIVLTQSPATLSLSPGERATLSCSASSSVSYMHWFQQKPGQSPKLLIYSTSNLASGIPARFS
GSGSGTDYTLTISSLEPEDFAVYYCQQRSSYPLTFGQGTKLEIK
The amino acid sequence of Hu147-VL3 is as shown in
SEQ ID NO: 24
EIVLTQSPATLSLSPGERATLSCSASSSVSYMHWFQQKPGQSPKLWIYSTSNLASGVPARFS
GSGSGTDYTLTISSLEPEDFAVYYCQQRSSYPLTFGQGTKLEIK
The amino acid sequence of Hu147-VL4 is as shown in
SEQ ID NO: 25
DIQMTQSPSSLSASVGDRVTITCSASSSVSYMHWFQQKPGKSPKLLIYSTSNLASGVPSRF
SGSGSGTDYTLTISSLQPEDFATYYCQQRSSYPLTFGQGTKLEIK
The amino acid sequence of Hu147-VL5 is as shown in
SEQ ID NO: 26
DIQLTQSPSSLSASVGDRVTITCSASSSVSYMHWFQQKPGKSPKLWIYSTSNLASGVPSRF
SGSGSGTDYTLTISSLQPEDFATYYCQQRSSYPLTFGQGTKLEIK
The amino acid sequence of Hu147-VH1 is as shown in
SEQ ID NO: 27
EVQLVESGGGLVQPGGSLRLSCAASGFTFSTYAMNWVRQAPGKGLEWVGRIRTKSNNY
ATYYADSVKDRFTISRDDSKNSLYLQMNSLKTEDTAVYYCVETGFAYWDQGTLVTVSS
The amino acid sequence of Hu147-VH2 is as shown in
SEQ ID NO: 28
EVQLVESGGGLVQPGGSLRLSCAASGFTFNTYAMNWVRQAPGKGLEWVARIRTKSNNYA
TYYADSVKDRFTISRDDSKNSLYLQMNSLKTEDTAVYYCVETGFAYWDQGTLVTVSS
The amino acid sequence of Hu147-VH3 is as shown in
SEQ ID NO: 29
EVQLVESGGGLVQPGGSLRLSCAASGFSFNTYAMNWVRQAPGKGLEWVARIRTKSNNYA
TYYADSVKDRFTISRDDSESSLYLQMNSLKTEDTAVYYCVETGFAYWDQGTLVTVSS.

Note:
The CDR sequences determined according to Kabat Criteria are underlined, and the sequences are arranged in the order of FR1-CDR1-FR2-CDR2-FR3-CDR3-FR4.

### 2. Humanization of Mab164

For murine antibody mab164, the light chain templates for humanization were composed of IGLV7-43*01 and IGLJ2*01, IGLV8-61*01 and IGLJ2*01, or IGLV1-40*02 and IGLJ2*01 respectively, and the heavy chain templates for humanization were composed of IGHV2-26*01 and IGHJ6*01, or IGHV4-31*02 and IGHJ6*01, respectively. Each of the CDRs of the murine antibody mab164 was grafted into its human template, and then the amino acids of the FR of the humanized antibody were subjected to back mutation. Among them, the back mutation(s) in the light chain variable region include one or more selected from the group consisting of 36V, 44F, 46G or 49G, and the back mutation(s) in the heavy chain variable region include one or more selected from the group consisting of 44G, 49G, 27F, 48L, 67L, 71K, 78V or 80F (the back mutation positions in the light and heavy chain variable regions were determined according to the Kabat numbering criteria), resulting in the humanized heavy and the light chain of mab164, with variable region sequences as follows:

The specific variable region sequences of the humanized antibodies of mAb164 are as follows:

The amino acid sequence of Hu164-VL7 is as shown in
SEQ ID NO: 30
EIWTQEPSLTVSPGGTVTLTCRSSIGAVTTSNYANWVQQKPGQAFRGLIGGTSNRAPWT
PARFSGSLLGGKAALTLSGVQPEDEAEYYCALWYSTHYVFGGGTKLTVL
The amino acid sequence of Hu164-VL8 is as shown in
SEQ ID NO: 31
ETWTQEPSFSVSPGGTVTLTCRSSIGAVTTSNYANWVQQTPGQAFRGLIGGTSNRAPGV
PDRFSGSILGNKAALTITGAQADDESDYYCALWYSTHYVFGGGTKLTVL
The amino acid sequence of Hu164-VL9 is as shown in
SEQ ID NO: 32
ESWTQPPSVSGAPGQRVTISCRSSIGAVTTSNYANWVQQLPGTAFKGLIGGTSNRAPGV
PDRFSGSKSGTSASLAITGLQAEDEADYYCALWYSTHYVFGGGTKLTVL
The amino acid sequence of Hu164-VH5 is as shown in
SEQ ID NO: 33
EVTLKESGPVLVKPTETLTLTCTVSGFSLSTFGVHWIRQPPGKALEWLAVIWRRGGTDYN
AAFMSRLTISKDTSKSQWLTMTNMDPVDTATYYCARDGGFDYWGQGTTVTVSS
The amino acid sequence of Hu164-VH6 is as shown in
SEQ ID NO: 34
EVTLKESGPVLVKPTETLTLTCTVSGFSLSTFGVHWIRQPPGKGLEWLGVIWRRGGTDY
NAAFMSRLTISKDTSKSQVVFTMTNMDPVDTATYYCARDGGFDYWGQGTTVTVSS
The amino acid sequence of Hu164-VH7 is as shown in
SEQ ID NO: 35
EVQLQESGPGLVKPSQTLSLTCTVSGFSISTFGVHWIRQHPGKGLEWIGVIWRRGGTDY
NAAFMSRVTISKDTSKNQVSLKLSSVTAADTAVYYCARDGGFDYWGQGTTVTVSS
The amino acid sequence of Hu164-VH8 is as shown in
SEQ ID NO: 36
EVQLQESGPGLVKPSQTLSLTCTVSGFSISTFGVHWIRQHPGKGLEWLGVIWRRGGTDY
NAAFMSRLTISKDTSKNQVSFKLSSVTAADTAVYYCARDGGFDYWGQGTTVTVSS.

Note:
In the above mentioned Hu164 antibody sequences, the underline represents CDR sequence, and the sequence order is FR1-CDR1-FR2-CDR2-FR3-CDR3-FR4, wherein the CDR sequences are defined according to Kabat criteria.

### 3. Humanization of Mab95

For murine antibody mab95, the light chain templates for humanization were IGKV3-20*02 and IGKV1-40*01, and the heavy chain template for humanization was IGHV1-3*01. Each of the CDRs of the murine antibody mab95 was grafted into its human template, and then the amino acids of the FR of the humanized antibody were subjected to back mutation. Among them, the back mutation(s) in the light chain variable region include one or more selected from the group consisting of 45P, 46W, 48Y, 69S or 70Y, and the back mutation(s) in the heavy chain variable region include one or more selected from the group consisting of 27F, 38K, 481, 67K, 68A, 70L or 72F (the back mutation positions in the light and heavy chain variable regions were determined according to the Kabat numbering criteria), resulting in the light chain variable regions and the heavy chain variable regions with different sequences. The humanized antibodies comprising the CDRs of mab95 were obtained, and the variable region sequences thereof are as follows:

The specific variable region sequences of the humanized antibodies of mAb95 are as follows:

The amino acid sequence of Hu95-VL1 is as shown in
SEQ ID NO: 77
EIVLTQSPATLSLSPGERATLSCRASSSVSYIHWYQQKPGQAPRPWIYATSNLASGIPARFSG
SGSGTDYTLTISRLEPEDFAVYYCQQWNSNPWTFGGGTKVEIK
The amino acid sequence of Hu95-VL2 is as shown in
SEQ ID NO: 78
EIVLTQSPATLSLSPGERATLSCRASSSVSYIHWYQQKPGQSPRPWIYATSNLASGVPARFS
GSGSGTSYTLTISRLEPEDFAVYYCQQWNSNPWTFGGGTKVEIK
The amino acid sequence of Hu95-VL3 is as shown in
SEQ ID NO: 79
ESVLTQPPSVSGAPGQRVTISCRASSSVSYIHWYQQLPGTAPKPWIYATSNLASGVPDRFS
GSKSGTSYSLAITGLQAEDEADYYCQQWNSNPWTFGGGTKVEIK
The amino acid sequence of Hu95-VL4 is as shown in
SEQ ID NO: 80
EIVLTQPPSVSGAPGQRVTISCRASSSVSYIHWYQQLPGTSPKPWIYATSNLASGVPDRFS
GSKSGTSYSLAITGLQAEDEADYYCQQWNSNPWTFGGGTKVEIK
The amino acid sequence of Hu95-VH1 is as shown in
SEQ ID NO: 81
EVQLVQSGAEVKKPGASVKVSCKASGFTFTNYDIHWVRQAPGQRLEWMGLVYPYNGGT
AYNQKFKDRVTLTFDTSASTAYMELSSLRSEDTAVYYCARWGLIPGTTSYFDVWGQGTTVTVSS
The amino acid sequence of Hu95-VH2 is as shown in
SEQ ID NO: 82
EVQLVQSGAEVKKPGASVKVSCKASGFTFTNYDIHWVRQAPGQRLEWIGLVYPYNGGT
AYNQKFKDRATLTFDTSASTAYMELSSLRSEDTAVYYCARWGLIPGTTSYFDVWGQGTTVTVSS
The amino acid sequence of Hu95-VH3 is as shown in
SEQ ID NO: 83
EVQLVQSGAEVKKPGASVKVSCKASGFTFTNYDIHWVKQAPGQRLEWMGLVYPYNGG
TAYNQKFKDKVTLTFDTSASTAYMELSSLRSEDTAVYYCARWGLIPGTTSYFDVWGQGTTVTVSS
The amino acid sequence of Hu95-VH4 is as shown in
SEQ ID NO: 84
EVQLVQSGAEVKKPGASVKVSCKASGFTFTNYDIHWVKQAPGQRLEWIGLVYPYNGGT
AYNQKFKDKATLTFDTSASTAYMELSSLRSEDTAVYYCARWGLIPGTTSYFDVWGQGTTVTVSS
The amino acid sequence of Hu95-VH5 is as shown in
SEQ ID NO: 85
EVQLVQSGAEVKKPGASVKVSCRASGFTFTNYAIHWVKQAPGQRLEWMGLVYPYTGGT
AYNQKFKDKVTLTFDTSASTAYMELSSLRSEDTAVYYCARWGMIPGTNSYFDVWGQGTTVTVSS.

Note:
The CDR sequences determined according to Kabat Criteria are underlined, and the sequences are arranged in the order of FR1-CDR1-FR2-CDR2-FR3-CDR3-FR4.

Among them, the CDR regions of Hu95-VH5 were further modified, wherein HCDR1 as shown in SEQ ID NO: 102 (NYAIH), HCDR2 as shown in SEQ ID NO: 103 (LVYPYTGGTAYNQKFKD), and HCDR3 as shown in SEQ ID NO: 104 (WGMIPGTNSYFDV).

### 4. Construction and Expression of Anti-CTGF Humanized Antibody

Primers were designed, VH/VL gene fragment of each humanized antibody was constructed by PCR and then inserted into the expression vector pHr (with a signal peptide and constant region gene (CH/CL) fragment) via homologous recombination to construct an expression vector for a full-length antibody VH-CH -pHr/VL-CL-pHr. For the humanized antibody, the constant region can be selected from the group consisting of the light chain constant region of human κ, λ chain, and can be selected from the group consisting of the heavy chain constant region of IgG1, IgG2, IgG3 or IgG4 or variant thereof. Non-limiting examples include optimizing the constant region of human IgG1, IgG2 or IgG4 to improve antibody's function. For example, the half-life of the antibody can be prolonged by point mutations in the constant region such as M252Y/S254T/T256E (YTE) site mutations, and the mutations such as S228P, F234A and L235A in the constant region.

An exemplary anti-CTGF humanized antibody constant region sequence is as follows:

The amino acid sequence of the heavy chain constant region is as shown in SEQ ID NO: 37 (the full-length antibody heavy chain is named with a suffix: w):

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK.

The amino acid sequence of the heavy chain constant region of the YTE engineered antibody is as shown in SEQ ID NO: 38 (the full-length antibody heavy chain is named with a suffix: y):

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLYITREPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK.

The amino acid sequence of the light chain kappa constant region of the anti-CTGF humanized antibody is as shown in SEQ ID NO: 39: (the full-length antibody heavy chain is named with a suffix: k):

RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

The amino acid sequence of the light chain lambda constant region of the anti-CTGF humanized antibody is as shown in SEQ ID NO: 40: (the full-length antibody heavy chain is named with a suffix: l):

GQPKANPTVTLFPPSSEELQANKATLVCLISDFYPGAVTVAWKADGSPVK
AGVETTKPSKQSNNKYAASSYLSLTPEQWKSHRSYSCQVTHEGSTVEKTV
APTEC.

The heavy chain variable region of the humanized antibody was linked to the heavy chain constant region as shown in SEQ ID NO: 37, and the light chain variable region of the humanized antibody was linked to the light chain kappa constant region as shown in SEQ ID NO: 39, resulting in a full-length humanized antibody derived from mab147, including:

TABLE 5

| | Hu147-VL1 | Hu147-VL2 | Hu147-VL3 | Hu147-VL4 | Hu147-VL5 |
|---|---|---|---|---|---|
| Hu147-VH1 | Hu147-11wk | Hu147-12wk | Hu147-13wk | Hu147-14wk | Hu147-15wk |
| Hu147-VH2 | Hu147-21wk | Hu147-22wk | Hu147-23wk | Hu147-24wk | Hu147-25wk |
| Hu147-VH3 | Hu147-31wk | Hu147-32wk | Hu147-33wk | Hu147-34wk | Hu147-35wk |

The heavy chain variable region of the humanized antibody was linked to the heavy chain constant region YTE variant as shown in SEQ ID NO: 38, and the light chain variable region of the humanized antibody was linked to the light chain kappa constant region as shown in SEQ ID NO: 39, resulting in a full-length humanized antibody derived from mab147, including:

TABLE 6

| | Hu147-VL1 | Hu147-VL2 | Hu147-VL3 | Hu147-VL4 | Hu147-VL5 |
|---|---|---|---|---|---|
| Hu147-VH1 | Hu147-11yk | Hu147-12yk | Hu147-13yk | Hu147-14yk | Hu147-15yk |
| Hu147-VH2 | Hu147-21yk | Hu147-22yk | Hu147-23yk | Hu147-24yk | Hu147-25yk |
| Hu147-VH3 | Hu147-31yk | Hu147-32yk | Hu147-33yk | Hu147-34yk | Hu147-35yk |

The heavy chain variable region of the humanized antibody was linked to the heavy chain constant region as shown in SEQ ID NO: 37, and the light chain variable region of the humanized antibody was linked to the light chain lambda constant region as shown in SEQ ID NO: 40, resulting in a full-length humanized antibody derived from mab164, including:

TABLE 7

| | Hu164-VH5 | Hu164-VH6 | Hu164-VH7 | Hu164-VH8 |
|---|---|---|---|---|
| Hu164-VL7 | Hu164-57wl | Hu164-67wl | Hu164-77wl | Hu164-87wl |
| Hu164-VL8 | Hu164-58wl | Hu164-68wl | Hu164-78wl | Hu164-88wl |
| Hu164-VL9 | Hu164-59wl | Hu164-69wl | Hu164-79wl | Hu164-89wl |

The heavy chain variable region of the humanized antibody was linked to the heavy chain constant region YTE variant as shown in SEQ ID NO: 38, and the light chain variable region of the humanized antibody was linked to the light chain lambda constant region as shown in SEQ ID NO: 40, resulting in a full-length humanized antibody derived from mab164, including:

TABLE 8

| | Hu164-VH5 | Hu164-VH6 | Hu164-VH7 | Hu164-VH8 |
|---|---|---|---|---|
| Hu164-VL7 | Hu164-57yl | Hu164-67yl | Hu164-77yl | Hu164-87yl |
| Hu164-VL8 | Hu164-58yl | Hu164-68yl | Hu164-78yl | Hu164-88yl |
| Hu164-VL9 | Hu164-59yl | Hu164-69yl | Hu164-79yl | Hu164-89yl |

The heavy chain variable region of the humanized antibody was linked to the heavy chain constant region as shown in SEQ ID NO: 37, and the light chain variable region of the humanized antibody was linked to the light chain kappa constant region as shown in SEQ ID NO: 39, resulting in a full-length humanized antibody derived from mab95, including:

TABLE 9

| | Hu95-VH1 | Hu95-VH2 | Hu95-VH3 | Hu95-VH4 | Hu95-VH5 |
|---|---|---|---|---|---|
| Hu95-VL1 | Hu95-11wk | Hu95-21wk | Hu95-31wk | Hu95-41wk | Hu95-51wk |
| Hu95-VL2 | Hu95-12wk | Hu95-22wk | Hu95-32wk | Hu95-42wk | Hu95-52wk |
| Hu95-VL3 | Hu95-13wk | Hu95-23wk | Hu95-33wk | Hu95-43wk | Hu95-53wk |
| Hu95-VL4 | Hu95-14wk | Hu95-24wk | Hu95-34wk | Hu95-44wk | Hu95-54wk |

The heavy chain variable region of the humanized antibody was linked to the heavy chain constant region YTE variant as shown in SEQ ID NO: 38, and the light chain variable region of the humanized antibody was linked to the light chain kappa constant region as shown in SEQ ID NO: 39, resulting in a full-length humanized antibody derived from mab95, including:

TABLE 10

| | Hu95-VH1 | Hu95-VH2 | Hu95-VH3 | Hu95-VH4 | Hu95-VH5 |
|---|---|---|---|---|---|
| Hu95-VL1 | Hu95-11yk | Hu95-21yk | Hu95-31yk | Hu95-41yk | Hu95-51yk |
| Hu95-VL2 | Hu95-12yk | Hu95-22yk | Hu95-32yk | Hu95-42yk | Hu95-52yk |
| Hu95-VL3 | Hu95-13yk | Hu95-23yk | Hu95-33yk | Hu95-43yk | Hu95-53yk |
| Hu95-VL4 | Hu95-14yk | Hu95-24yk | Hu95-34yk | Hu95-44yk | Hu95-54yk |

Exemplary amino acid sequences of full-length anti-CTGF antibody are as follows:

TABLE 11
The light chain or heavy chain of full-length antibody

| Full-length antibody | |
|---|---|
| light chain or heavy | |
| chain (SEQ ID NO) | Amino acid sequence |
| Ch147 heavy chain | EVQLVESGGGLVQPEGSLKLSCAASGFSFNTYAMNWVRQAPG |
| (SEQ ID NO: 41) | KGLEWVARIRTKSNNYATYYADSVKDRFTISRDDSESMLYLQMN |
| | NLKTEDTAMYYCVETGFAYWDQGTLVTVSAASYKGPSVFPEAP |
| | SSKSTSGGTAALGCLVKDYFPEPVTVSWNSGALTSGVHTFP |
| | AVLQSSGLYSLSSVVTVPSSSLGTQTYICNVNHKPSNTKVD |
| | KKVEPKSCDKTHTCPPCPAPELLGGPSVFLFPPKPKDTLMIS |
| | RTPEVTCVVVDVSHEDPEVKFNWYVDGVEVHNAKTKPRE |
| | EQYNSTYRVVSVLTVLHQDWLNGKEYKCKVSNKALPAPIE |
| | KTISKAKGQPREPQVYTLPPSRDELTKNQVSLTCLVKGFYPS |
| | DIAVEWESNGQPENNYKTTPPVLDSDGSFFLYSKLTVDKSR |
| | WQQGNVFSCSVMHEALHNHYTQKSLSLSPGK |
| Ch147 light chain | QIVLTQSPAIMSASPGEKVTITCSASSSVSYMHWFQQKPGTSPKL |
| (SEQ ID NO: 42) | WIYSTSNLASGVPARFSGSGSGTSYSLTISRMEAEDAATYYCQQR |
| | SSYPLTFGAGTKLELKRTVAAPSVFIFPPSDEQLKSGTASVVC |
| | LLNNFYPREAKVQWKVDNALQSGNSQESVTEQDSKDSTYS |
| | LSSTLTLSKADYEKHKVYACEVTHQGLSSPVTKSFNRGEC |
| Full-length heavy | EVQLVESGGGLVQPGGSLRLSCAASGFTFSTYAMNWVRQAPG |
| chain for humanized | KGLEWVGRIRTKSNNYATYYADSVKDRFTISRDDSKNSLYLQMN |
| antibodies Hu147- | SLKTEDTAVYYCVETGFAYWDQGTLVTVSSASTfAGPSVFPEAPS |
| 11wk, Hu147-12wk, | SKSTSGGTAALGCLVKDYFPEPVTVSWNSGALTSGVHTFPA |
| Hu 147-13wk, | VLQSSGLYSLSSVVTVPSSSLGTQTYICNVNHKPSNTKVDK |
| Hu147-14wk and | KVEPKSCDKTHTCPPCPAPELLGGPSVFLFPPKPKDTLMISR |
| Hu 147-15wk | TPEVTCVVVDVSHEDPEVKFNWYVDGVEVHNAKTKPREE |
| (SEQ ID NO: 43) | QYNSTYRVVSVLTVLHQDWLNGKEYKCKVSNKALPAPIEK |
| | TISKAKGQPREPQVYTLPPSRDELTKNQVSLTCLVKGFYPSD |
| | IAVEWESNGQPENNYKTTPPVLDSDGSFFLYSKLTVDKSRW |
| | QQGNVFSCSVMHEALHNHYTQKSLSLSPGK |
| Full-length heavy | EVQLVESGGGLVQPGGSLRLSCAASGFTFNTYAMNWVRQAPG |
| chain for humanized | KGLEWVARIRTKSNNYATYYADSVKDRFTISRDDSKNSLYLQMN |
| antibodies Hu147- | SLKTEDTAVYYCVETGFAYWDOGTLVTVSSASTKGPSVFPEAPS |
| 21wk, Hu147-22wk, | SKSTSGGTAALGCLVKDYFPEPVTVSWNSGALTSGVHTFPA |
| Hu147-23wk, | VLQSSGLYSLSSVVTVPSSSLGTQTYICNVNHKPSNTKVDK |
| Hu147-24wk and | KVEPKSCDKTHTCPPCPAPELLGGPSVFLFPPKPKDTLMISR |
| Hu147-24wk | TPEVTCVVVDVSHEDPEVKFNWYVDGVEVHNAKTKPREE |
| (SEQ ID NO: 44) | QYNSTYRVVSVLTVLHQDWLNGKEYKCKVSNKALPAPIEK |
| | TISKAKGQPREPQVYTLPPSRDELTKNQVSLTCLVKGFYPSD |
| | IAVEWESNGQPENNYKTTPPVLDSDGSFFLYSKLTVDKSRW |
| | QQGNVFSCSVMHEALHNHYTQKSLSLSPGK |
| Full-length heavy | EVQLVESGGGLVQPGGSLRLSCAASGFSFNTYAMNWVRQAPG |
| chain for humanized | KGLEWVARIRTKSNNYATYYADSVKDRFTISRDDSESSLYLQMNS |
| antibodies Hu147- | LKTEDTAVYYCVETGFAYWDQGTLVTVSSASYKGPSVFPEAPSS |
| 31wk, Hu147-32wk, | KSTSGGTAALGCLVKDYFPEPVTVSWNSGALTSGVHTFPAV |
| Hu147-33wk, | LQSSGLYSLSSVVTVPSSSLGTQTYICNVNHKPSNTKVDKK |
| Hu147-34wk and | VEPKSCDKTHTCPPCPAPELLGGPSVFLFPPKPKDTLMISRTP |
| Hu147-35wk | EVTCVVVDVSHEDPEVKFNWYVDGVEVHNAKTKPREEQY |
| (SEQ ID NO: 45) | NSTYRVVSVLTVLHQDWLNGKEYKCKVSNKALPAPIEKTIS |
| | KAKGQPREPQVYTLPPSRDELTKNQVSLTCLVKGFYPSDIA |
| | VEWESNGQPENNYKTTPPVLDSDGSFFLYSKLTVDKSRWQ |
| | QGNVFSCSVMHEALHNHYTQKSLSLSPGK |
| Full-length heavy | EVQLVESGGGLVQPGGSLRLSCAASGFTFSTYAMNWVRQAPG |
| chain for humanized | KGLEWVGRIRTKSNNYATYYADSVKDRFTISRDDSKNSLYLOMN |
| antibodies Hu147- | SLKTEDTAVYYCVETGFAYWDOGTLVTVSSASTKGPSVFPEAPS |
| 11yk, Hu147-12yk, | SKSTSGGTAALGCLVKDYFPEPVTVSWNSGALTSGVHTFPA |
| Hu147-13yk, | VLQSSGLYSLSSVVTVPSSSLGTQTYICNVNHKPSNTKVDK |
| Hu147-14yk and | KVEPKSCDKTHTCPPCPAPELLGGPSVFLFPPKPKDTLYITR |
| Hu147-15yk | EPEVTCVVVDVSHEDPEVKFNWYVDGVEVHNAKTKPREE |
| (SEQ ID NO: 46) | QYNSTYRVVSVLTVLHQDWLNGKEYKCKVSNKALPAPIEK |
| | TISKAKGQPREPQVYTLPPSRDELTKNQVSLTCLVKGFYPSD |
| | IAVEWESNGQPENNYKTTPPVLDSDGSFFLYSKLTVDKSRW |
| | QQGNVFSCSVMHEALHNHYTQKSLSLSPGK |
| Full-length heavy | EVQLVESGGGLVQPGGSLRLSCAASGFTFNTYAMNWVRQAPG |
| chain for humanized | KGLEWVARIRTKSNNYATYYADSVKDRFTISRDDSKNSLYLQMN |
| antibodies Hu147- | SLKTEDTAVYYCVETGFAYWDQGTLVTVSSASTKGPSVFPEAPS |
| 21yk, Hu147-22yk, | SKSTSGGTAALGCLVKDYFPEPVTVSWNSGALTSGVHTFPA |
| Hu147-23yk, | VLQSSGLYSLSSVVTVPSSSLGTQTYICNVNHKPSNTKVDK |
| Hu147-24yk and | KVEPKSCDKTHTCPPCPAPELLGGPSVFLFPPKPKDTLYITR |
| Hu147-24yk | EPEVTCVVVDVSHEDPEVKFNWYVDGVEVHNAKTKPREE |
| (SEQ ID NO: 47) | QYNSTYRVVSVLTVLHQDWLNGKEYKCKVSNKALPAPIEK |
| | TISKAKGQPREPQVYTLPPSRDELTKNQVSLTCLVKGFYPSD |
| | IAVEWESNGQPENNYKTTPPVLDSDGSFFLYSKLTVDKSRW |
| | QQGNVFSCSVMHEALHNHYTQKSLSLSPGK |
| Full-length heavy | EVQLVESGGGLVQPGGSLRLSCAASGFSFNTYAMNWVRQAPG |
| chain for humanized | KGLEWVARIRTKSNNYATYYADSVKDRFTISRDDSESSLYLOMNS |
| antibodies Hu147- | LKTEDTAVYYCVETGFAYWDQGTLVTVSSASYKGPSVFPEAPSS |
| 31yk, Hu147-32yk, | KSTSGGTAALGCLVKDYFPEPVTVSWNSGALTSGVHTFPAV |
| Hu147-33yk, | LQSSGLYSLSSVVTVPSSSLGTQTYICNVNHKPSNTKVDKK |
| Hu147-34yk and | VEPKSCDKTHTCPPCPAPELLGGPSVFLFPPKPKDTLYITREP |
| Hu147-35yk | EVTCVVVDVSHEDPEVKFNWYVDGVEVHNAKTKPREEQY |
| (SEQ ID NO: 48) | NSTYRVVSVLTVLHQDWLNGKEYKCKVSNKALPAPIEKTIS |
| | KAKGQPREPQVYTLPPSRDELTKNQVSLTCLVKGFYPSDIA |
| | VEWESNGQPENNYKTTPPVLDSDGSFFLYSKLTVDKSRWQ |
| | QGNVFSCSVMHEALHNHYTQKSLSLSPGK |
| Full-lengthlight | EIVLTQSPATLSLSPGERATLSCSASSSVSYMHWFQQKPGQSPRL |
| chain for humanized | LIYSTSNLASGIPARFSGSGSGTDYTLTISSLEPEDFAVYYCQQRSS |
| antibodies Hu147- | YPLTFGQGTKLEIKRTVAAPSVFIFPPSDEQLKSGTASVVCLL |
| 11wk, Hu147-21wk | NNFYPREAKVQWKVDNALQSGNSQESVTEQDSKDSTYSLS |
| and Hu 147-31wk | STLTLSKADYEKHKVYACEVTHQGLSSPVTKSFNRGEC |
| (SEQ ID NO: 49) | (It is also the full-length light chain for Hu147-11yk, |
| | Hu147-21yk and Hu147-31yk) |
| Full-lengthlight | EIVLTQSPATLSLSPGERATLSCSASSSVSYMHWFQQKPGQSPKL |
| chain for humanized | LIYSTSNLASGIPARFSGSGSGTDYTLTISSLEPEDFAVYYCQQRSS |
| antibodies Hu147- | YPLTFGQGTKLEIKRTVAAPSVFIFPPSDEQLKSGTASVVCLL |
| 12wk, Hu147-22wk | NNFYPREAKVQWKVDNALQSGNSQESVTEQDSKDSTYSLS |
| and Hu 147-32wk | STLTLSKADYEKHKVYACEVTHQGLSSPVTKSFNRGEC |
| (SEQ ID NO: 50) | (It is also the full-length light chain for Hu147-12yk, |
| | Hu147-22yk and Hui47-32yk) |
| Full-lengthlight | EIVLTQSPATLSLSPGERATLSCSASSSVSYMHWFQQKPGQSPKL |
| chain for humanized | WIYSTSNLASGVPARFSGSGSGTDYTLTISSLEPEDFAVYYCQQRS |
| antibodies Hu147- | SYPLTFGQGTKLEIKRTVAAPSVFIFPPSDEQLKSGTASVVCL |
| 13wk, Hu147-23wk | LNNFYPREAKVQWKVDNALQSGNSQESVTEQDSKDSTYSL |
| and Hu147-33wk | S STLTLSKADYEKHKVYACEVTHQGLS SP VTKSFNRGEC |
| (SEQ ID NO: 51) | (It is also the full-length light chain for Hu147-13yk, |
| | Hu147-23yk and Hu147-33yk) |
| Full-lengthlight | DIQMTQSPSSLSASVGDRVTITCSASSSVSYMHWFQQKPGKSPK |
| chain for humanized | LLIYSTSNLASGVPSRFSGSGSGTDYTLTISSLQPEDFATYYCQQR |
| antibodies Hu147- | SSYPLTFGOGTKLEIKRTVAAPSVFIFPPSDEQLKSGTASVVCL |
| 14wk, Hu147-24wk | LNNFYPREAKVQWKVDNALQSGNSQESVTEQDSKDSTYSL |
| and Hu 147-34wk | S STLTLSKADYEKHKVYACEVTHQGLS SP VTKSFNRGEC |
| (SEQ ID NO: 52) | (It is also the full-length light chain for Hu147-14yk, |
| | Hu147-24yk and Hu147-34yk) |
| Full-lengthlight | DIQLTQSPSSLSASVGDRVTITCSASSSVSYMHWFQQKPGKSPK |
| chain for humanized | LWIYSTSNLASGVPSRFSGSGSGTDYTLTISSLQPEDFATYYCQQ |
| antibodies Hu147- | RSSYPLTFGOGTKLEIKRTVAAPSVFIFPPSDEQLKSGTASVVC |
| 15wk, Hu147-25wk | LLNNFYPREAKVQWKVDNALQSGNSQESVTEQDSKDSTYS |
| and Hu 147-3 5wk | LSSTLTLSKADYEKHKVYACEVTHQGLSSPVTKSFNRGEC |
| (SEQ ID NO: 53) | (It is also the full-length light chain for Hu147-15yk, |
| | Hu147-25yk and Hu147-35yk) |
| Ch164 heavy chain | QVQLKQSGPGLVQPSQSLSITCTVSGFSLTTFGVHWIRQSPGK |
| (SEQ ID NO: 54) | GLEWLGVIWRRGGTDYNAAFMSRLSITKDNSRSHVFFKMTSLQ |
| | TDDSAIYYCARDGGFDYWGQGTTLIVSSASYKGYSNYYENPSS |
| | KSTSGGTAALGCLVKDYFPEPVTVSWNSGALTSGVHTFPAV |
| | LQSSGLYSLSSVVTVPSSSLGTQTYICNVNHKPSNTKVDKK |
| | VEPKSCDKTHTCPPCPAPELLGGPSVFLFPPKPKDTLMISRTP |
| | EVTCVVVDVSHEDPEVKFNWYVDGVEVHNAKTKPREEQY |
| | NSTYRVVSVLTVLHQDWLNGKEYKCKVSNKALPAPIEKTIS |
| | KAKGQPREPQVYTLPPSRDELTKNQVSLTCLVKGFYPSDIA |
| | VEWESNGQPENNYKTTPPVLDSDGSFFLYSKLTVDKSRWQ |
| | QGNVFSCSVMHEALHNHYTQKSLSLSPGK |
| Ch164 light chain | QAVVTQESALTTSPGGTVILTCRSSIGAVTTSNYANWVQEKPDH |
| (SEQ ID NO: 55) | LFTGLIGGTSNRAPGVPVRFSGSLIGKAALTITGAQAEDDAMY |
| | FCALWYSTHYVFGGGTKVTVLGQPKANPTVTLFPPSSEELQA |
| | NKATLVCLISDFYPGAVTVAWKADGSPVKAGVETTKPSKQS |
| | NNKYAASSYLSLTPEQWKSHRSYSCQVTHEGSTVEKTVAPT |
| | EC |
| Full-length heavy | EVTLKESGPVLVKPTETLTLTCTVSGFSLSTFGVHWIRQPPGKA |
| chain for Hu 164- | LEWLAVIWRRGGTDYNAAFMSRLTISKDTSKSQVVLTMTNMDP |
| 57wl, Hu164-58wl | VDTATYYCARDGGFDYWGQGTTVTVSSASYKGPSVPLAPSS |
| and Hu 164-59wl | KSTSGGTAALGCLVKDYFPEPVTVSWNSGALTSGVHTFPAV |
| (SEQ ID NO: 56) | LQSSGLYSLSSVVTVPSSSLGTQTYICNVNHKPSNTKVDKK |
| | VEPKSCDKTHTCPPCPAPELLGGPSVFLFPPKPKDTLMISRTP |
| | EVTCVVVDVSHEDPEVKFNWYVDGVEVHNAKTKPREEQY |
| | NSTYRVVSVLTVLHQDWLNGKEYKCKVSNKALPAPIEKTIS |
| | KAKGQPREPQVYTLPPSRDELTKNQVSLTCLVKGFYPSDIA |
| | VEWESNGQPENNYKTTPPVLDSDGSFFLYSKLTVDKSRWQ |
| | QGNVFSCSVMHEALHNHYTQKSLSLSPGK |
| Full-length heavy | EVTLKESGPVLVKPTETLTLTCTVSGFSLSTFGVHWIRQPPGKG |
| chain for Hu 164- | LEWLGVIWRRGGTDYNAAFMSRLTISKDTSKSQVVFTMTNMD |
| 67wl, Hu164-68wl | PVDTATYYCARDGGFDYWGQGTTVTVSSASYKGPSVFPLAPSS |
| and Hu164-69wl | KSTSGGTAALGCLVKDYFPEPVTVSWNSGALTSGVHTFPAV |
| (SEQ ID NO: 57) | LQSSGLYSLSSVVTVPSSSLGTQTYICNVNHKPSNTKVDKK |
| | VEPKSCDKTHTCPPCPAPELLGGPSVFLFPPKPKDTLMISRTP |
| | EVTCVVVDVSHEDPEVKFNWYVDGVEVHNAKTKPREEQY |
| | NSTYRVVSVLTVLHQDWLNGKEYKCKVSNKALPAPIEKTIS |
| | KAKGQPREPQVYTLPPSRDELTKNQVSLTCLVKGFYPSDIA |
| | VEWESNGQPENNYKTTPPVLDSDGSFFLYSKLTVDKSRWQ |
| | QGNVFSCSVMHEALHNHYTQKSLSLSPGK |
| Full-length heavy | EVQLQESGPGLVKPSQTLSLTCTVSGFSISTFGVHWIRQHPGK |
| chain for Hu 164- | GLEWIGVIWRRGGTDYNAAFMSRVTISKDTSKNQVSLKLSSVTA |
| 77wl, Hu164-78wl | ADTAVYYCARDGGFDYWGQGTTVTVSSASYKGYSNYYENPSS |
| and Hu164-79wl | KSTSGGTAALGCLVKDYFPEPVTVSWNSGALTSGVHTFPAV |
| (SEQ ID NO: 58) | LQSSGLYSLSSVVTVPSSSLGTQTYICNVNHKPSNTKVDKK |
| | VEPKSCDKTHTCPPCPAPELLGGPSVFLFPPKPKDTLMISRTP |
| | EVTCVVVDVSHEDPEVKFNWYVDGVEVHNAKTKPREEQY |
| | NSTYRVVSVLTVLHQDWLNGKEYKCKVSNKALPAPIEKTIS |
| | KAKGQPREPQVYTLPPSRDELTKNQVSLTCLVKGFYPSDIA |
| | VEWESNGQPENNYKTTPPVLDSDGSFFLYSKLTVDKSRWQ |
| | QGNVFSCSVMHEALHNHYTQKSLSLSPGK |
| Full-length heavy | EVQLQESGPGLVKPSQTLSLTCTVSGFSISTFGVHWIRQHPGK |
| chain for Hu 164- | GLEWLGVIWRRGGTDYNAAFMSRLTISKDTSKNQVSFKLSSVTA |
| 87, Hu164-88wl | ADTAVYYCARDGGFDYWGQGTTVTVSSASYKGYSNYYENPSS |
| and Hu164-89wl | KSTSGGTAALGCLVKDYFPEPVTVSWNSGALTSGVHTFPAV |
| (SEQ ID NO: 59) | LQSSGLYSLSSVVTVPSSSLGTQTYICNVNHKPSNTKVDKK |
| | VEPKSCDKTHTCPPCPAPELLGGPSVFLFPPKPKDTLMISRTP |
| | EVTCVVVDVSHEDPEVKFNWYVDGVEVHNAKTKPREEQY |
| | NSTYRVVSVLTVLHQDWLNGKEYKCKVSNKALPAPIEKTIS |
| | KAKGQPREPQVYTLPPSRDELTKNQVSLTCLVKGFYPSDIA |
| | VEWESNGQPENNYKTTPPVLDSDGSFFLYSKLTVDKSRWQ |
| | QGNVFSCSVMHEALHNHYTQKSLSLSPGK |
| Full-length heavy | EVTLKESGPVLVKPTETLTLTCTVSGFSLSTFGVHWIRQPPGKA |
| chain for Hu164- | LEWLAVIWRRGGTDYNAAFMSRLTISKDTSKSQVVLTMTNMDP |
| 57yl, Hu164-58yl | VDTATYYCARDGGFDYWGQGTTVTVSSKSYKGYSNYYENPSS |
| and Hu164-59yl | KSTSGGTAALGCLVKDYFPEPVTVSWNSGALTSGVHTFPAV |
| (SEQ ID NO: 60) | LQSSGLYSLSSVVTVPSSSLGTQTYICNVNHKPSNTKVDKK |
| | VEPKSCDKTHTCPPCPAPELLGGPSVFLFPPKPKDTLYITREP |
| | EVTCVVVDVSHEDPEVKFNWYVDGVEVHNAKTKPREEQY |
| | NSTYRVVSVLTVLHQDWLNGKEYKCKVSNKALPAPIEKTIS |
| | KAKGQPREPQVYTLPPSRDELTKNQVSLTCLVKGFYPSDIA |
| | VEWESNGQPENNYKTTPPVLDSDGSFFLYSKLTVDKSRWQ |
| | QGNVFSCSVMHEALHNHYTQKSLSLSPGK |
| Full-length heavy | EVTLKESGPVLVKPTETLTLTCTVSGFSLSTFGVHWIRQPPGKG |
| chain for Hu164- | LEWLGVIWRRGGTDYNAAFMSRLTISKDTSKSQWFIMTNMD |
| 67yl, Hu164-68yl | PVDTATYYCARDGGFDYWGQGTTVTVSSASYKGYSNYYENPSS |
| and Hu164-69yl | KSTSGGTAALGCLVKDYFPEPVTVSWNSGALTSGVHTFPAV |
| (SEQIDNO: 61) | LQSSGLYSLSSVVTVPSSSLGTQTYICNVNHKPSNTKVDKK |
| | VEPKSCDKTHTCPPCPAPELLGGPSVFLFPPKPKDTLYITREP |
| | EVTCVVVDVSHEDPEVKFNWYVDGVEVHNAKTKPREEQY |
| | NSTYRVVSVLTVLHQDWLNGKEYKCKVSNKALPAPIEKTIS |
| | KAKGQPREPQVYTLPPSRDELTKNQVSLTCLVKGFYPSDIA |
| | VEWESNGQPENNYKTTPPVLDSDGSFFLYSKLTVDKSRWQ |
| | QGNVFSCSVMHEALHNHYTQKSLSLSPGK |
| Full-length heavy | EVQLQESGPGLVKPSQTLSLTCTVSGFSISTFGVHWIRQHPGK |
| chain for Hu164- | GLEWIGVIWRRGGTDYNAAFMSRVTISKDTSKNQVSLKLSSVTA |
| 77yl, Hu164-78yl | ADTAVYYCARDGGFDYWGQGTTVTVSSASYKGYSNYYENPSS |
| and Hu164-79yl | KSTSGGTAALGCLVKDYFPEPVTVSWNSGALTSGVHTFPAV |
| (SEQ ID NO: 62) | LQSSGLYSLSSVVTVPSSSLGTQTYICNVNHKPSNTKVDKK |
| | VEPKSCDKTHTCPPCPAPELLGGPSVFLFPPKPKDTLYITREP |
| | EVTCVVVDVSHEDPEVKFNWYVDGVEVHNAKTKPREEQY |
| | NSTYRVVSVLTVLHQDWLNGKEYKCKVSNKALPAPIEKTIS |
| | KAKGQPREPQVYTLPPSRDELTKNQVSLTCLVKGFYPSDIA |
| | VEWESNGQPENNYKTTPPVLDSDGSFFLYSKLTVDKSRWQ |
| | QGNVFSCSVMHEALHNHYTQKSLSLSPGK |
| Full-length heavy | EVQLQESGPGLVKPSQTLSLTCTVSGFSISTFGVHWIRQHPGK |
| chain for Hu164- | GLEWLGVIWRRGGTDYNAAFMSRLTISKDTSKNQVSFKLSSVTA |
| 87yl, Hu164-88yl | ADTAVYYCARDGGFDYWGQGTTVTVSSASYKGYSNYYENPSS |
| and Hu164-89yl | KSTSGGTAALGCLVKDYFPEPVTVSWNSGALTSGVHTFPAV |
| (SEQ ID NO: 63) | LQSSGLYSLSSVVTVPSSSLGTQTYICNVNHKPSNTKVDKK |
| | VEPKSCDKTHTCPPCPAPELLGGPSVFLFPPKPKDTLYITREP |
| | EVTCVVVDVSHEDPEVKFNWYVDGVEVHNAKTKPREEQY |
| | NSTYRVVSVLTVLHQDWLNGKEYKCKVSNKALPAPIEKTIS |
| | KAKGQPREPQVYTLPPSRDELTKNQVSLTCLVKGFYPSDIA |
| | VEWESNGQPENNYKTTPPVLDSDGSFFLYSKLTVDKSRWQ |
| | QGNVFSCSVMHEALHNHYTQKSLSLSPGK |
| Full-lengthlight | ETVVTQEPSLTVSPGGTVTLTCRSSIGAVTTSNYANWVQQKPGQ |
| chain for Hu164- | AFRGLIGGTSNRAPWTPARFSGSLLGGKAALTLSGVQPEDEAEY |
| 57wl, Hu164-67wl, | YCALWYSTHYVFGGGTKLTVLGQYKANPTVTLFPPSSEELQA |
| Hu164-77wl and | NKATLVCLISDFYPGAVTVAWKADGSPVKAGVETTKPSKQS |
| Hu164-87wl | NNKYAASSYLSLTPEQWKSHRSYSCQVTHEGSTVEKTVAPT |
| (SEQ ID NO: 64) | EC |
| | (It is also the full-length light chain for Hu164-57yl, |
| | Hu164-67yl, Hu164-77yl and Hu164-87yl) |
| Full-lengthlight | ETVVTQEPSFSVSPGGTVTLTCRSSIGAVTTSNYANWVQQTPGQ |
| chain for Hu164- | AFRGLIGGTSNRAPGVPDRFSGSILGNKAALTITGAQADDESDY |
| 58wl, Hu164-68wl, | YCALWYSTHYVFGGGTKLTVLGQPKANPTVTLFPPSSEELQA |
| Hu164-78wl and | NKATLVCLISDFYPGAVTVAWKADGSPVKAGVETTKPSKQS |
| Hu164-88wl | NNKYAASSYLSLTPEQWKSHRSYSCQVTHEGSTVEKTVAPT |
| (SEQ ID NO: 65) | EC |
| | (It is also the full-length light chain for Hu164-58yl, |
| | Hu164-68yl, Hu164-78yl and Hu164-88yl) |
| Full-lengthlight | ESVVTQPPSVSGAPGQRVTISCRSSIGAVTTSNYANWVQQLPGTA |
| chain for Hu164- | FKGLIGGTSNRAPGVPDRFSGSKSGTSASLAITGLQAEDEADYY |
| 59wl, Hu164-69wl, | CALWYSTHYVFGGGTKLTVLGQPKKANPTVTLFPPSSEELQAN |
| Hu164-79wl and | KATLVCLISDFYPGAVTVAWKADGSPVKAGVETTKPSKQSN |
| Hu164-89wl | NKYAASSYLSLTPEQWKSHRSYSCQVTHEGSTVEKTVAPTE |
| (SEQ ID NO: 66) | C |
| | (It is also the full-length light chain for Hu164-59yl, |
| | Hu164-69yl, Hu164-79yl and Hu164-89yl) |
| Ch95 heavy chain | EVLLQQSGPVLVKPGPSVKISCKASGFTFTNYDIHWVKQSHGK |
| (SEQ ID NO: 86) | SLEWIGLVYPYNGGTAYNQKFKDKATLTFDTSSSTAYMELNSLTS |
| | EDSAFYYCARWGLIPGTTSYFDVWGTGTTVTVSSASYKGPSVF |
| | PLAPSSKSTSGGTAALGCLVKDYFPEPVTVSWNSGALTSGV |
| | HTFPAVLQSSGLYSLSSVVTVPSSSLGTQTYICNVNHKPSNT |
| | KVDKKVEPKSCDKTHTCPPCPAPELLGGPSVFLFPPKPKDT |
| | LMISRTPEVTCVVVDVSHEDPEVKFNWYVDGVEVHNAKT |
| | KPREEQYNSTYRVVSVLTVLHQDWLNGKEYKCKVSNKAL |
| | PAPIEKTISKAKGQPREPQVYTLPPSRDELTKNQVSLTCLVK |
| | GFYPSDIAVEWESNGQPENNYKTTPPVLDSDGSFFLYSKLTV |
| | DKSRWQQGNVFSCSVMHEALHNHYTQKSLSLSPGK |
| Ch95 light chain | QIVLSQSPPILSASPGEKVTMTCRASSSVSYIHWYQQKPRSSPKP |
| (SEQ ID NO: 87) | WIYATSNLASGVPARFSGSGSGTSYSLTISRVEAEDAATYYCQQW |
| | NSNPWTFGGGTRLEIKRTVAAPSVFIFPPSDEQLKSGTASVVC |
| | LLNNFYPREAKVQWKVDNALQSGNSQESVTEQDSKDSTYS |
| | LSSTLTLSKADYEKHKVYACEVTHQGLSSPVTKSFNRGEC |
| Full-length heavy | EVQLVQSGAEVKKPGASVKVSCKASGFTFTNYDIHWVRQAPG |
| chain for Hu95- | QRLEWMGLVYPYNGGTAYNQKFKDRVTLTFDTSASTAYMELSS |
| 11wk, Hu95-12wk, | LRSEDTAVYYCARWGLIPGTTSYFDVWGQGTTVTVSSASYKGY |
| Hu95-13wk and | SVFPLAPSSKSTSGGTAALGCLVKDYFPEPVTVSWNSGALT |
| Hu95-14wk | SGVHTFPAVLQSSGLYSLSSWTVPSSSLGTQTYICNVNHKP |
| (SEQ ID NO: 88) | SNTKVDKKVEPKSCDKTHTCPPCPAPELLGGPSVFLFPPKP |
| | KDTLMISRTPEVTCVVVDVSHEDPEVKFNWYVDGVEVHN |
| | AKTKPREEQYNSTYRVVSVLTVLHQDWLNGKEYKCKVSN |
| | KALPAPIEKTISKAKGQPREPQVYTLPPSRDELTKNQVSLTC |
| | LVKGFYPSDIAVEWESNGQPENNYKTTPPVLDSDGSFFLYS |
| | KLTVDKSRWQQGNVFSCSVMHEALHNHYTQKSLSLSPGK |
| Full-length heavy | EVQLVQSGAEVKKPGASVKVSCKASGFTFTNYDIHWVRQAPG |
| chain for Hu95- | QRLEWIGLVYPYNGGTAYNQKFKDRATLTFDTSASTAYMELSSL |
| 21wk, Hu95-22wk, | RSEDTAVYYCARWGLIPGTTSYFDVWGQGTTVTVSSASYKGYS |
| Hu95-23wk and | VFPLAPSSKSTSGGTAALGCLVKDYFPEPVTVSWNSGALTS |
| Hu95-24wk | GVHTFPAVLQSSGLYSLSSVVTVPSSSLGTQTYICNVNHKPS |
| (SEQ ID NO: 89) | NTKVDKKVEPKSCDKTHTCPPCPAPELLGGPSVFLFPPKPK |
| | DTLMISRTPEVTCVVVDVSHEDPEVKFNWYVDGVEVHNA |
| | KTKPREEQYNSTYRVVSVLTVLHQDWLNGKEYKCKVSNK |
| | ALPAPIEKTISKAKGQPREPQVYTLPPSRDELTKNQVSLTCL |
| | VKGFYPSDIAVEWESNGQPENNYKTTPPVLDSDGSFFLYSK |
| | LTVDKSRWQQGNVFSCSVMHEALHNHYTQKSLSLSPGK |
| Full-length heavy | EVQLVQSGAEVKKPGASVKVSCKASGFTFTNYDIHWVKQAPG |
| chain for Hu95- | QRLEWMGLVYPYNGGTAYNQKFKDKVTLTFDTSASTAYMELSS |
| 31wk, Hu95-32wk, | LRSEDTAVYYCARWGLIPGTTSYFDVWGQGTTVTVSSASYKGY |
| Hu95-33wk and | SVFPLAPSSKSTSGGTAALGCLVKDYFPEPVTVSWNSGALT |
| Hu95-34wk | SGVHTFPAVLQSSGLYSLSSWTVPSSSLGTQTYICNVNHKP |
| (SEQ ID NO: 90) | SNTKVDKKVEPKSCDKTHTCPPCPAPELLGGPSVFLFPPKP |
| | KDTLMISRTPEVTCVVVDVSHEDPEVKFNWYVDGVEVHN |
| | AKTKPREEQYNSTYRVVSVLTVLHQDWLNGKEYKCKVSN |
| | KALPAPIEKTISKAKGQPREPQVYTLPPSRDELTKNQVSLTC |
| | LVKGFYPSDIAVEWESNGQPENNYKTTPPVLDSDGSFFLYS |
| | KLTVDKSRWQQGNVFSCSVMHEALHNHYTQKSLSLSPGK |
| Full-length heavy | EVQLVQSGAEVKKPGASVKVSCKASGFTFTNYDIHWVKQAPG |
| chain for Hu95- | QRLEWIGLVYPYNGGTAYNQKFKDKATLTFDTSASTAYMELSSL |
| 41wk, Hu95-42wk, | RSEDTAVYYCARWGLIPGTTSYFDVWGQGTTVTVSSASYKGYS |
| Hu95-43wk and | VFPLAPSSKSTSGGTAALGCLVKDYFPEPVTVSWNSGALTS |
| Hu95-44wk | GVHTFPAVLQSSGLYSLSSVVTVPSSSLGTQTYICNVNHKPS |
| (SEQ ID NO: 91) | NTKVDKKVEPKSCDKTHTCPPCPAPELLGGPSVFLFPPKPK |
| | DTLMISRTPEVTCVVVDVSHEDPEVKFNWYVDGVEVHNA |
| | KTKPREEQYNSTYRVVSVLTVLHQDWLNGKEYKCKVSNK |
| | ALPAPIEKTISKAKGQPREPQVYTLPPSRDELTKNQVSLTCL |
| | VKGFYPSDIAVEWESNGQPENNYKTTPPVLDSDGSFFLYSK |
| | LTVDKSRWQQGNVFSCSVMHEALHNHYTQKSLSLSPGK |
| Full-length heavy | EVQLVQSGAEVKKPGASVKVSCRASGFTFTNYAIHWVKQAPGQ |
| chain for Hu95- | RLEWMGLVYPYTGGTAYNQKFKDKVTLTFDTSASTAYMELSSLR |
| 51wk, Hu95-52wk, | SEDTAVYYCARWGMIPGTNSYFDVWGQGTTVTVSSASYKGYSN |
| Hu95-53wk and | FPLAPSSKSTSGGTAALGCLVKDYFPEPVTVSWNSGALTSG |
| Hu95-54wk | VHTFPAVLQSSGLYSLSSVVTVPSSSLGTQTYICNVNHKPSN |
| (SEQ ID NO: 92) | TKVDKKVEPKSCDKTHTCPPCPAPELLGGPSVFLFPPKPKD |
| | TLMISRTPEVTCVVVDVSHEDPEVKFNWYVDGVEVHNAK |
| | TKPREEQYNSTYRVVSVLTVLHQDWLNGKEYKCKVSNKA |
| | LPAPIEKTISKAKGQPREPQVYTLPPSRDELTKNQVSLTCLV |
| | KGFYPSDIAVEWESNGQPENNYKTTPPVLDSDGSFFLYSKLT |
| | VDKSRWQQGNVFSCSVMHEALHNHYTQKSLSLSPGK |
| Full-length heavy | EVQLVQSGAEVKKPGASVKVSCKASGFTFTNYDIHWVRQAPG |
| chain for Hu95- | QRLEWMGLVYPYNGGTAYNQKFKDRVTLTFDTSASTAYMELSS |
| llyk, Hu95-12yk, | LRSEDTAVYYCARWGLIPGTTSYFDVWGQGTTVTVSSASYKGY |
| Hu95-13yk and | SVFPLAPSSKSTSGGTAALGCLVKDYFPEPVTVSWNSGALT |
| Hu95-14yk | SGVHTFPAVLQSSGLYSLSSWTVPSSSLGTQTYICNVNHKP |
| (SEQ ID NO: 93) | SNTKVDKKVEPKSCDKTHTCPPCPAPELLGGPSVFLFPPKP |
| | KDTLYITREPEVTCVVVDVSHEDPEVKFNWYVDGVEVHNA |
| | KTKPREEQYNSTYRVVSVLTVLHQDWLNGKEYKCKVSNK |
| | ALPAPIEKTISKAKGQPREPQVYTLPPSRDELTKNQVSLTCL |
| | VKGFYPSDIAVEWESNGQPENNYKTTPPVLDSDGSFFLYSK |
| | LTVDKSRWQQGNVFSCSVMHEALHNHYTQKSLSLSPGK |
| Full-length heavy | EVQLVQSGAEVKKPGASVKVSCKASGFTFTNYDIHWVRQAPG |
| chain for Hu95- | QRLEWIGLVYPYNGGTAYNQKFKDRATLTFDTSASTAYMELSSL |
| 21yk, Hu95-22yk, | RSEDTAVYYCARWGLIPGTTSYFDVWGQGTTVTVSSASYKGYS |
| Hu95-23yk and | VFPLAPSSKSTSGGTAALGCLVKDYFPEPVTVSWNSGALTS |
| Hu95-24yk | GVHTFPAVLQSSGLYSLSSVVTVPSSSLGTQTYICNVNHKPS |
| (SEQ ID NO: 94) | NTKVDKKVEPKSCDKTHTCPPCPAPELLGGPSVFLFPPKPK |
| | DTLYITREPEVTCVVVDVSHEDPEVKFNWYVDGVEVHNAK |
| | TKPREEQYNSTYRVVSVLTVLHQDWLNGKEYKCKVSNKA |
| | LPAPIEKTISKAKGQPREPQVYTLPPSRDELTKNQVSLTCLV |
| | KGFYPSDIAVEWESNGQPENNYKTTPPVLDSDGSFFLYSKLT |
| | VDKSRWQQGNVFSCSVMHEALHNHYTQKSLSLSPGK |
| Full-length heavy | EVQLVQSGAEVKKPGASVKVSCKASGFTFTNYDIHWVKQAPG |
| chain for Hu95- | QRLEWMGLVYPYNGGTAYNQKFKDKVTLTFDTSASTAYMELSS |
| 31yk, Hu95-32yk, | LRSEDTAVYYCARWGLIPGTTSYFDVWGQGTTVTVSSASYKGY |
| Hu95-33yk and | SVFPLAPSSKSTSGGTAALGCLVKDYFPEPVTVSWNSGALT |
| Hu95-34yk | SGVHTFPAVLQSSGLYSLSSWTVPSSSLGTQTYICNVNHKP |
| (SEQ ID NO: 95) | SNTKVDKKVEPKSCDKTHTCPPCPAPELLGGPSVFLFPPKP |
| | KDTLYITREPEVTCVVVDVSHEDPEVKFNWYVDGVEVHNA |
| | KTKPREEQYNSTYRVVSVLTVLHQDWLNGKEYKCKVSNK |
| | ALPAPIEKTISKAKGQPREPQVYTLPPSRDELTKNQVSLTCL |
| | VKGFYPSDIAVEWESNGQPENNYKTTPPVLDSDGSFFLYSK |
| | LTVDKSRWQQGNVFSCSVMHEALHNHYTQKSLSLSPGK |
| Full-length heavy | EVQLVQSGAEVKKPGASVKVSCKASGFTFTNYDIHWVKQAPG |
| chain for Hu95- | QRLEWIGLVYPYNGGTAYNQKFKDKATLTFDTSASTAYMELSSL |
| 41yk, Hu95-42yk, | RSEDTAVYYCARWGLIPGTTSYFDVWGQGTTVTVSSASYKGYS |
| Hu95-43yk and | VFPLAPSSKSTSGGTAALGCLVKDYFPEPVTVSWNSGALTS |
| Hu95-44yk | GVHTFPAVLQSSGLYSLSSVVTVPSSSLGTQTYICNVNHKPS |
| (SEQ ID NO: 96) | NTKVDKKVEPKSCDKTHTCPPCPAPELLGGPSVFLFPPKPK |
| | DTLYITREPEVTCVVVDVSHEDPEVKFNWYVDGVEVHNAK |
| | TKPREEQYNSTYRVVSVLTVLHQDWLNGKEYKCKVSNKA |
| | LPAPIEKTISKAKGQPREPQVYTLPPSRDELTKNQVSLTCLV |
| | KGFYPSDIAVEWESNGQPENNYKTTPPVLDSDGSFFLYSKLT |
| | VDKSRWQQGNVFSCSVMHEALHNHYTQKSLSLSPGK |
| Full-length heavy | EVQLVQSGAEVKKPGASVKVSCRASGFTFTNYAIHWVKQAPGQ |
| chain for Hu95- | RLEWMGLVYPYTGGTAYNQKFKDKVTLTFDTSASTAYMELSSLR |
| 51yk, Hu95-52yk, | SEDTAVYYCARWGMIPGTNSYFDVWGQGTTVTVSSASYKGYSN |
| Hu95-53yk and | FPLAPSSKSTSGGTAALGCLVKDYFPEPVTVSWNSGALTSG |
| Hu95-54yk | VHTFPAVLQSSGLYSLSSVVTVPSSSLGTQTYICNVNHKPSN |
| (SEQ ID NO: 97) | TKVDKKVEPKSCDKTHTCPPCPAPELLGGPSVFLFPPKPKD |
| | TLYITREPEVTCVVVDVSHEDPEVKFNWYVDGVEVHNAKT |
| | KPREEQYNSTYRVVSVLTVLHQDWLNGKEYKCKVSNKAL |
| | PAPIEKTISKAKGQPREPQVYTLPPSRDELTKNQVSLTCLVK |
| | GFYPSDIAVEWESNGQPENNYKTTPPVLDSDGSFFLYSKLTV |
| | DKSRWQQGNVFSCSVMHEALHNHYTQKSLSLSPGK |
| Full-lengthlight | EIVLTQSPATLSLSPGERATLSCRASSSVSYIHWYQQKPGQAPRP |
| chain for Hu95- | WIYATSNLASGIPARFSGSGSGTDYTLTISRLEPEDFAVYYCQQW |
| 11wk, Hu95-21wk, | NSNPWTFGGGTKVEIKRTVAAPSVFIFPPSDEQLKSGTASVVC |
| Hu95-31wk, Hu95- | LLNNFYPREAKVQWKVDNALQSGNSQESVTEQDSKDSTYS |
| 41wk and Hu95- | LSSTLTLSKADYEKHKVYACEVTHQGLSSPVTKSFNRGEC |
| 51wk | (It is also the full-length light chain for Hu95-11yk, |
| (SEQ ID NO: 98) | Hu95-21yk, Hu95-31yk, Hu95-41yk and Hu95-51yk) |
| Full-lengthlight | EIVLTQSPATLSLSPGERATLSCRASSSVSYIHWYQQKPGQSPRP |
| chain for Hu95- | WIYATSNLASGVPARFSGSGSGTSYTLTISRLEPEDFAVYYCQQW |
| 12wk, Hu95-22wk, | NSNPWTFGGGTKVEIKRTVAAPSVFIFPPSDEQLKSGTASVVC |
| Hu95-32wk, Hu95- | LLNNFYPREAKVQWKVDNALQSGNSQESVTEQDSKDSTYS |
| 42wk and Hu95- | LSSTLTLSKADYEKHKVYACEVTHQGLSSPVTKSFNRGEC |
| 52wk | (It is also the full-length light chain for Hu95-12yk, |
| (SEQ ID NO: 99) | Hu95-22yk, Hu95-32yk, Hu95-42yk and Hu95-52yk) |
| Full-lengthlight | ESVLTQPPSVSGAPGQRVTISCRASSSVSYIHWYQQLPGTAPKP |
| chain for Hu95- | WIYATSNLASGVPDRFSGSKSGTSYSLAITGLQAEDEADYYCQQ_ |
| 13wk, Hu95-23wk, | WNSNPWTFGGGTKVEIKRTVAAPSVFIFPPSDEOLKSGTASV |
| Hu95-33wk, Hu95- | VCLLNNFYPREAKVQWKVDNALQSGNSQESVTEQDSKDS |
| 43wk and Hu95- | TYSLSSTLTLSKADYEKHKVYACEVTHQGLSSPVTKSFNRG |
| 53wk | EC |
| (SEQ ID NO: 100) | (It is also the full-length light chain for Hu95-13yk, |
| | Hu95-23yk, Hu95-33yk, Hu95-43yk and Hu95-53yk) |
| Full-lengthlight | EIVLTQPPSVSGAPGQRVTISCRASSSVSYIHWYQQLPGTSPKPW |
| chain for Hu95- | IYATSNLASGVPDRFSGSKSGTSYSLAITGLQAEDEADYYCQQW |
| 14wk, Hu95-24wk, | NSNPWTFGGGTKVEIKRTVAAPSVFIFPPSDEQLKSGTASVVC |
| Hu95-34wk, Hu95- | LLNNFYPREAKVQWKVDNALQSGNSQESVTEQDSKDSTYS |
| 44wk and Hu95- | LSSTLTLSKADYEKHKVYACEVTHQGLSSPVTKSFNRGEC |
| 54wk | (It is also the full-length light chain for Hu95-14yk, |
| (SEQ ID NO: 101) | Hu95-24yk, Hu95-34yk, Hu95-44yk and Hu95-54yk) |

The sequences of the positive control CTGF antibody mAb 1 (from CN1829740B, with the antibody name of pamrevlumab, also known as FG-3019) are as follows:

Heavy chain:
(SEQ ID NO: 67)
EGQLVQSGGGLVHPGGSLRLSCAGSGFTFSSYGMHWVRQAPGKGLEWVSGIGTGGGTY
STDSVKGRFTISRDNAKNSLYLQMNSLRAEDMAVYYCARGDYYGSGSFFDCWGQGTLV
TVSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKRVEPKSCDKTHTCPPCPAPELL
GGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPS
REEMTKNQVSLTCLVKGFYPSDIAVEWESNGQPENNYKTTPPVLDSDGSFFLYSKLTVDK
SRWQQGNVFSCSVMHEALHNHYTQKSLSLSPGK;
Light chain:
(SEQ ID NO: 68)
DIQMTQSPSSLSASVGDRVTITCRASQGISSWLAWYQQKPEKAPKSLIYAASSLQS
GVPSRFSGSGSGTDFTLTISSLQPEDFATYYCQQYNSYPPTFGQGTKLEIKRTVAAPSVFIFP
PSDEQLKSGTASVVCLLNNFYPREAKVQWKVDNALQSGNSQESVTEQDSKDSTYSLSST
LTLSKADYEKHKVYACEVTHQGLSSPVTKSFNRGEC.

### Example 3. Detection of binding activity of CTGF antibody

### I. ELISA Assay of the Binding of CTGF Antibody to Human CTGF Protein

The binding activity of anti-human CTGF antibody to human CTGF protein was tested by ELISA assay and Biacore. A 96-well microtiter plate was directly coated with the CTGF recombinant protein. The signal strength upon the addition of the antibody was used to determine the binding activity of the antibody to CTGF. The specific experimental procedures were as follows:

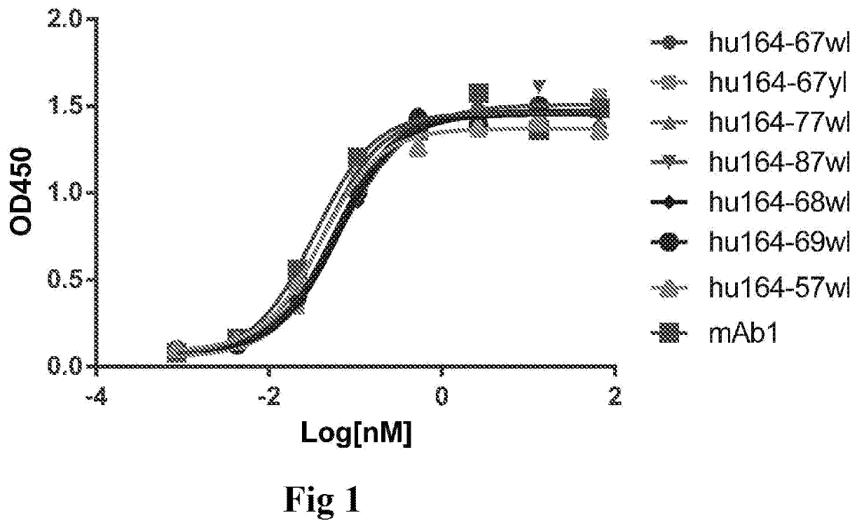

The CTGF protein (R&D, Cat No. 9190-CC) was diluted with PBS, pH 7.4 (Shanghai Yuanpei, Cat No. B320) buffer to a concentration of 0.2 μg/ml, 50 μl/well of which was added into the 96-well microtiter plate (Corning, Cat No. CLS3590-100EA), and incubated in an incubator at 37° C. for 2 hours or at 4° C. overnight (16-18 hours). The liquid was removed, 250 μl/ well blocking solution (5% skim milk (BD skim milk, Cat No.232100) diluted in PBS) was added, and incubated in an incubator at 37° C. for 3 hours or at 4° C. overnight (16-18 hours) for blocking. After the blocking was finished, the blocking solution was removed, the plate was washed for 5 times with PBST buffer (PBS containing 0.1% Tween-20, pH 7.4), 50 μl/well of various concentrations of each of the test antibodies (antibodies purified from hybridoma or humanized antibodies) diluted with sample dilution solution were added, and incubated in an incubator at 37° C. for 1 hours. After the incubation was finished, the plate was washed for 3 times with PBST, 50 μl/well of HRP-labeled goat anti-mouse secondary antibody (Jackson Immuno Research, Cat No. 115-035-003) or goat anti-human secondary antibody (Jackson Immuno Research, Cat No. 109-035-003) diluted with sample dilution solution was added, and incubated at 37° C. for 1 hour. The plate was washed for 3 times with PBST, 50 μl/well of TMB chromogenic substrate (KPL, Cat No. 52-00-03) was added, incubated at room temperature for 5-10min, and 50 μl/well of 1 M H₂SO₄ was added to stop the reaction. The absorbance value was read by a microplate reader (Molecular Devices, VERSA max) at a wavelength of 450 nm, and the data was analyzed with GraphPad Prism 5. The binding of CTGF antibody to human CTGF protein was calculated as EC50 value. The experimental results are shown in FIG. 1 and Table 12.

TABLE 12
ELISA results of the binding of CTGF
antibody to human CTGF protein

| | EC50 (nM) representing the |
|---|---|
| | binding to human CTGF |
| Test sample | protein, in ELISA assay |
| Hu147-11wk | 0.045 |
| Hu147-12wk | 0.046 |
| Hu147-13wk | 0.043 |
| Hu147-14wk | 0.073 |
| Hu147-15wk | 0.034 |
| Hu147-21wk | 0.067 |
| Hu147-22wk | 0.056 |
| Hu147-23wk | 0.058 |
| Hu147-24wk | 0.048 |
| Hu147-31wk | 0.056 |
| Hu147-32wk | 0.063 |
| Hu147-33wk | 0.054 |
| Hu147-34wk | 0.134 |
| Hu164-57wl | 0.044 |
| Hu164-67wl | 0.044 |
| Hu164-67yl | 0.060 |
| Hu164-77wl | 0.055 |
| Hu164-87wl | 0.055 |
| Hu164-68wl | 0.069 |
| Hu164-69wl | 0.063 |
| mAb1 | 0.067 |
| Hu95-51yk | 0.080 |

The experimental results show that the humanized full-length anti-CTGF antibodies of the present disclosure all have excellent binding effect with the human CTGF protein.

### II. ELISA Assay of the Binding of CTGF Antibody to Cynomolgus Monkey CTGF Protein

The binding activity of anti-monkey CTGF antibody to monkey CTGF protein was tested by ELISA assay. A 96-well microtiter plate was directly coated with anti-mFc protein, and then the cyno-CTGF protein with Fc tag was bound to the plate, and then the antibody was added. The signal strength upon the addition of the antibody was used to determine the binding activity of the antibody to cyno-CTGF. The specific experimental procedures were as follows:

The anti-mFc Protein (Sigma, Cat No. M4280) was diluted with PBS, pH 7.4 (Shanghai Yuanpei, Cat No. B320) buffer to a concentration of 2 μg/ml, which was added into the 96-well microtiter plate (Corning, Cat No. CLS3590-100EA) at 50 μl/well, and incubated in an incubator at 37° C. for 2 hours. The liquid was removed, 250 μl/well blocking solution (5% skim milk (BD skim milk, Cat No.232100) diluted in PBS) was added, and incubated in an incubator at 37° C. for 3 hours or at 4° C. overnight (16-18 hours) for blocking. After the blocking was finished, the blocking solution was removed, the plate was washed for 3 times with PBST buffer (PBS containing 0.1% Tween-20, pH 7.4), 50 μl of 1 μg/ml cyno-CTGF-mFc protein was added into each well, incubated in an incubator at 37° C. for 1 hour, and then the plate was washed for 3 times with PBST buffer (PBS containing 0.1% Tween-20, pH 7.4). 50 μl/well of various concentrations of each of the test antibodies (antibodies purified from hybridoma or humanized antibodies) diluted with sample dilution solution were added, and incubated in an incubator at 37° C. for 1 hours. After the incubation was finished, the plate was washed for 3 times with PBST, 50 μl/well of HRP-labeled goat anti-mouse secondary antibody (Jackson Immuno Research, Cat No. 115-035-003) or goat anti-human secondary antibody (Jackson Immuno Research, Cat No. 109-035-003) diluted with sample dilution solution was added, and incubated at 37° C. for 1 hour. The plate was washed for 5 times with PBST, 50 μl/well of TMB chromogenic substrate (KPL, Cat No. 52-00-03) was added, incubated at room temperature for 5-10 min, and 50 μl/well of 1 M H₂SO₄ was added to stop the reaction. The absorbance value was read by a microplate reader (Molecular Devices, VERSA max) at a wavelength of 450 nm, and the data was analyzed with GraphPad Prism 5. The binding of CTGF antibody to cyno CTGF protein was calculated as EC50 value. The experimental results are shown in Table 13.

TABLE 13
ELISA results of the binding of CTGF
antibody to cyno CTGF protein

| Test sample | EC50 (nM) |
|---|---|
| Hu147-11wk | 0.052 |
| Hu147-12wk | 0.062 |
| Hu147-13wk | 0.043 |
| Hu147-14wk | 0.077 |
| Hu147-15wk | 0.053 |
| Hu147-21wk | 0.053 |
| Hu147-22wk | 0.073 |
| Hu147-23wk | 0.063 |
| Hu147-24wk | 0.080 |
| Hu147-31wk | 0.070 |
| Hu147-32wk | 0.079 |
| Hu147-33wk | 0.044 |
| Hu147-34wk | 0.144 |
| Hu95-51yk | 0.059 |
| Hu164-57wl | 0.029 |
| Hu164-67wl | 0.028 |
| Hu164-77wl | 0.038 |
| Hu164-87wl | 0.034 |
| Hu164-68wl | 0.036 |
| Hu164-69wl | 0.032 |
| mAb1 | 0.086 |

The experimental results show that the humanized anti-CTGF antibodies of the present disclosure all have excellent binding effect with the monkey CTGF protein.

### III. ELISA Assay of the Binding of CTGF Antibody to Mouse CTGF Protein

The binding activity of anti-mouse CTGF antibody to mouse CTGF protein was tested by ELISA assay. A 96-well microtiter plate was directly coated with the mouse CTGF fusion protein. The signal strength upon the addition of the antibody was used to determine the binding activity of the antibody to mouse CTGF. The specific experimental procedures were as follows:

The mouse CTGF-his protein was diluted with PBS, pH 7.4 (Shanghai Yuanpei, Cat No. B320) buffer to a concentration of 0.5 μg/ml, added into the 96-well microtiter plate (Corning, Cat No. CLS3590-100EA) at 50 μl/well, and incubated in an incubator at 37° C. for 2 hours. The liquid was removed, 250 μl/well blocking solution (5% skim milk (BD skim milk, Cat No. 232100) diluted in PBS) was added, and incubated in an incubator at 37° C. for 3 hours or at 4° C. overnight (16-18 hours) for blocking. After the blocking was finished, the blocking solution was removed, the plate was washed for 3 times with PBST buffer (PBS containing 0.1% Tween-20, pH 7.4), 50 μl/well of various concentrations of each of the test antibodies (antibodies purified from hybridoma or humanized antibodies) diluted with sample dilution solution were added, and incubated in an incubator at 37° C. for 1 hours. After the incubation was finished, the plate was washed for 3 times with PBST, 50 μl/well of HRP-labeled goat anti-mouse secondary antibody (Jackson Immuno Research, Cat No. 115-035-003) or goat anti-human secondary antibody (Jackson Immuno Research, Cat No. 109-035-003) diluted with sample dilution solution was added, and incubated at 37° C. for 1 hour. The plate was washed for 5 times with PBST, 50 μl/well of TMB chromogenic substrate (KPL, Cat No. 52-00-03) was added, incubated at room temperature for 5-10min, and 50 μl/well of 1 M H₂SO₄ was added to stop the reaction. The absorbance value was read by a microplate reader (Molecular Devices, VERSA max) at a wavelength of 450 nm, and the data was analyzed with GraphPad Prism 5. The binding of CTGF antibody to mouse CTGF protein was calculated as EC50 value. The experimental results are shown in Table 14.

TABLE 14
ELISA results of the binding of CTGF
antibody to mouse CTGF protein

| Test sample | EC50 (nM) |
|---|---|
| Hu147-11wk | 0.228 |
| Hu147-12wk | 0.249 |
| Hu147-13wk | 0.127 |
| Hu147-14wk | 0.431 |
| Hu147-15wk | 0.159 |
| Hu147-21wk | 0.247 |
| Hu147-22wk | 0.336 |
| Hu147-23wk | 0.200 |
| Hu147-24wk | 0.263 |
| Hu147-31wk | 0.320 |
| Hu147-32wk | 0.349 |
| Hu147-33wk | 0.258 |
| Hu147-34wk | 0.664 |
| Hu164-57wl | 0.041 |
| Hu164-67wl | 0.049 |
| Hu164-77wl | 0.058 |
| Hu164-87wl | 0.067 |
| Hu164-68wl | 0.065 |
| Hu164-69wl | 0.059 |
| mAb1 | 0.077 |

The experimental results show that the humanized full-length anti-CTGF antibodies of the present disclosure all have excellent binding effect with the mouse CTGF protein.

### Example 4. Function-blocking Assay of CTGF Antibody

In this assay, Biacore instrument was used to detect the activity of the humanized anti-CTGF antibody to be tested to block the function of human TGF-β1.

First, the antigen human CTGF was immobilized onto a CMS biosensor chip (Cat. # BR-1005-30, GE) by amino coupling, with an immobilization level of 1500 RU, and then a high concentration of CTGF antibody (150 μg/ml) was allowed to flow through the surface of the chip for 150 s; 50 nM TGF-β1 protein was injected for 120 s, and the reaction signals were detected in real time with the Biacore T200 instrument to generate a binding and dissociation curve. After the dissociation was completed in each of the experimental cycles, the biosensor chip was washed and regenerated with regeneration buffer.

The resulting experimental data were statistically analyzed with GE Biacore T200 Evaluation version 3.0 software.

Blocking efficiency=[1−(response value of the sample/response value of the blank control)]×100%.

The specific results are shown in Table 15.

TABLE 15
Function-blocking assay of CTGF antibody

| | Function-blocking |
|---|---|
| Test sample | percentage (%) |
| Hu147-11wk | 50.8% |
| Hu147-12wk | 51.6% |
| Hu147-13wk | 53.3% |
| Hu147-14wk | 50.9% |
| Hu147-15wk | 52.3% |
| Hu147-21wk | 51.6% |
| Hu147-22wk | 52.3% |
| Hu147-23wk | 54.6% |
| Hu147-24wk | 52.8% |
| Hu147-31wk | 55.0% |
| Hu147-32wk | 55.2% |
| Hu147-33wk | 57.3% |
| Hu147-34wk | 56.2% |
| Hu164-67wl | 55.5% |
| Hu164-67yl | 53.9% |
| mAb1 | 41.3% |

The experimental results show that the humanized anti-CTGF antibodies of the present disclosure all have high function-blocking activity.

### Determination of the Affinity of Anti-CTGF Antibody by Biacore

In this experiment, Biacore instrument was used to determine the affinity of the humanized anti-CTGF antibodies to be tested with human CTGF and mouse CTGF.

A certain amount of each of the test antibodies was affinity-captured with Protein A biosensor chip (Cat. # 29127556, GE), and then a certain concentration of human or mouse CTGF was allowed to flow through the surface of the chip. The reaction signals were detected in real time with Biacore instrument to generate a binding and dissociation curve. After the dissociation was completed in each cycle, the biosensor chip was washed and regenerated with Glycine-HCl regeneration solution pH 1.5 (Cat. # BR-1003-54, GE). The running buffer for the assay was 1×HBS-EP buffer solution (Cat. # BR-1001-88, GE).

The resulting experimental data were fitted against the (1:1) Langmuir model using GE Biacore T200 Evaluation version 3.0 software, and the affinity values were obtained, as shown in Table 16 and 17 for details.

TABLE 16
The reaction affinity of CTGF antibody to human CTGF protein

| Antibody | ka (1/Ms) | kd (1/s) | KD (M) |
|---|---|---|---|
| Ch147 | 1.49E+07 | 6.93E−03 | 4.66E−10 |
| Ch164 | 6.62E+07 | 8.83E−03 | 1.33E−10 |
| Ch95 | 7.85E+06 | 3.02E−02 | 3.85E−09 |
| Hu147-11wk | 2.98E+07 | 3.36E−02 | 1.13E−09 |
| Hu147-12wk | 3.41E+07 | 3.30E−02 | 9.69E−10 |
| Hu147-13wk | 2.97E+07 | 2.40E−02 | 8.08E−10 |
| Hu147-14wk | 4.89E+07 | 5.54E−02 | 1.13E−09 |
| Hu147-21wk | 1.66E+07 | 1.62E−02 | 9.74E−10 |
| Hu147-22wk | 1.40E+07 | 1.42E−02 | 1.01E−09 |
| Hu147-23wk | 1.37E+07 | 1.12E−02 | 8.18E−10 |
| Hu147-31wk | 1.78E+07 | 1.59E−02 | 8.94E−10 |
| Hu147-32wk | 1.46E+07 | 1.33E−02 | 9.10E−10 |
| Hu147-33wk | 1.45E+07 | 1.04E−02 | 7.13E−10 |
| Hu147-34wk | 1.47E+07 | 1.55E−02 | 1.05E−09 |
| Hu164-57wl | 4.26E+07 | 2.94E−03 | 6.91E−11 |
| Hu164-67wl | 5.17E+07 | 1.96E−03 | 3.79E−11 |
| Hu164-67yl | 3.43E+07 | 2.27E−03 | 6.62E−11 |
| Hu164-77wl | 5.15E+07 | 1.76E−02 | 3.43E−10 |
| Hu164-87wl | 1.05E+08 | 2.04E−02 | 1.94E−10 |
| Hu164-68wl | 3.84E+07 | 1.16E−02 | 3.02E−10 |
| Hu164-69wl | 8.71E+07 | 2.80E−02 | 3.22E−10 |
| Hu95-21wk | 4.09E+06 | 3.03E−02 | 7.39E−09 |
| Hu95-31wk | 5.59E+06 | 3.26E−02 | 5.84E−09 |
| Hu95-41wk | 7.70E+06 | 4.08E−02 | 5.29E−09 |
| Hu95-42wk | 3.32E+06 | 1.91E−02 | 5.74E−09 |
| Hu95-51yk | 2.84E+07 | 1.02E−03 | 3.58E−11 |

TABLE 17
The reaction affinity of CTGF antibody to mouse CTGF protein

| Antibody | ka (1/Ms) | kd (1/s) | KD (M) |
|---|---|---|---|
| Ch147 | 3.12E+07 | 3.84E−02 | 1.23E−09 |
| Ch164 | 1.23E+08 | 1.01E−02 | 8.22E−11 |
| Ch95 | 1.38E+07 | 2.35E−02 | 1.71E−09 |
| Hu147-11wk | 1.20E+08 | 3.74E−01 | 3.11E−09 |
| Hu147-12wk | 3.75E+07 | 1.16E−01 | 3.09E−09 |
| Hu147-13wk | 5.73E+07 | 1.46E−01 | 2.55E−09 |
| Hu147-23wk | 8.18E+07 | 1.73E−01 | 2.12E−09 |
| Hu147-31wk | 3.83E+07 | 1.07E−02 | 2.79E−09 |
| Hu147-32wk | 1.37E+08 | 3.43E−01 | 2.49E−09 |
| Hu147-33wk | 9.01E+07 | 1.74E−01 | 1.93E−09 |
| Hu164-57wl | 7.35E+07 | 2.37E−03 | 3.22E−11 |
| Hu164-67wl | 6.36E+07 | 1.17E−03 | 1.84E−11 |
| Hu164-67yl | 5.34E+07 | 1.94E−03 | 3.63E−11 |
| Hu164-77wl | 5.98E+07 | 8.83E−03 | 1.48E−10 |
| Hu164-87wl | 5.24E+07 | 4.40E−03 | 8.40E−11 |
| Hu164-68wl | 5.70E+07 | 7.60E−03 | 1.33E−10 |
| Hu164-69wl | 6.17E+07 | 7.74E−03 | 1.26E−10 |
| Hu95-21wk | 5.29E+06 | 2.11E−02 | 3.99E−09 |
| Hu95-31wk | 7.84E+06 | 2.25E−02 | 2.87E−09 |
| Hu95-41wk | 1.39E+07 | 3.39E−02 | 2.44E−09 |
| Hu95-42wk | 5.66E+06 | 1.37E−02 | 2.42E−09 |
| Hu95-51yk | 2.56E+07 | 5.16E−04 | 2.02E−11 |

The experimental results show that the chimeric antibodies Ch147 and Ch164 and the humanized anti-CTGF antibodies derived from the murine antibodies mAb147 and mAb164 all can bind to human and mouse CTGF with high affinity. Neither the replacements nor the back mutation(s) present in the human germline antibody FR regions interferes with the affinity of the humanized antibody, and some modifications can even enhance the affinity of the antibody to the antigen.

### Example 5. The Assay of CTGF Antibody for In Vitro Inhibiting the Migration of C2C12 Cells Induced by TGFβ

C2C12 cells (mouse myoblasts, Cell Bank of Chinese Academy of Sciences, #GNM26) were digested with 0.25% trypsin (Gibco, #25200-072), centrifuged and resuspended in serum-free DMEM medium (Gibco, #10564-029). The cell-antibody mixture and TGFβ1-antibody mixture were prepared with serum-free DMEM medium, in which the cell density was 2×10⁵/ml, the concentration of the recombinant human TGFβ1 (Cell signaling Technology, #8915LC) was 10 ng/ml, and the concentration of the antibody to be tested was 30 μl/ml.

The cover and filter membrane of the ChemoTx ® Disposable Chemotaxis System (Neuro Probe, #106-8) were opened, and the TGFβ-antibody mixture was added into the lower chamber, 30 μl per well, 4 pairs of wells for each group. The filter membrane was covered, and the cell-antibody mixture was added onto the membrane, 50 μl per well, 4 pairs of wells for each group. The cover was closed and placed in an incubator (37° C., 5% CO₂).

48 hours after incubation, the liquid on the filter membrane was removed with a clean paper towel, the filter membrane was opened, 10 μl of pre-cooled 0.25% trypsin was added to each well into the lower chamber, and the filter membrane was covered. 5 minutes after digestion, the samples were centrifuged at 1000 rpm for 1 minute. The filter membrane was opened, 20 μl Cell Titer-Glo solution (Promega, #G7573) was added to each well into the lower chamber, and incubated at room temperature for 10 minutes. The liquid was transfered to a 384-well white bottom plate (Thermo Scientific, #267462), and the plate was read with a microplate reader (BMG, #PheraStar) by chemiluminescence. Data were analyzed and processed with Graphpad Prism 6.

Inhibition rate=[(average value of TGFβ−average value of sample group)/(average value of TGFβ group−average value of untreated group)]×100%

The experimental results are shown in Table 18.

TABLE 18
Results showing the inhibitory effect of CTGF antibody
on the migration of C2C12 cells in vitro

| | Inhibition percentage of C2C12 cell |
|---|---|
| Test sample | migration in vitro (%) |
| Hu147-11wk | 72.2% |
| Hu147-33wk | 63.8% |
| Hu164-57wl | 75.4% |
| Hu164-67wl | 78.8% |
| Hu164-67yl | 76.5% |
| Hu164-77wl | 72.8% |
| Hu164-87wl | 76.4% |
| Hu164-68wl | 76.3% |
| Hu164-69wl | 74.2% |
| Hu95-51yk | 80.0% |
| mAb1 | 63.0% |

The results show that the humanized anti-CTGF antibodies of the present disclosure can inhibit the migration ability of C2C12 cells in vitro induced by TGFβ1, and show stronger inhibitory effect on the migration ability of C2C12 cells when compared to that of the positive control antibody mAb1.

### Example 6. The assay Showing the In Vitro Inhibitory Effect of CTGF Antibody on the Migration of PANC1 Cells Induced by TGFβ

PANC-1 cells (human pancreatic cancer cells, ATCC, #CRL-1469) were digested with 0.25% trypsin (Gibco, #25200-072), centrifuged and resuspended with DMEM medium (Gibco, #10564-029) containing 0.1% fetal bovine serum (Gibco, #10099-141). The cell-antibody mixture and TGFβ-antibody mixture were prepared with DMEM medium containing 0.1% fetal bovine serum, in which the cell density was 4×10⁵/ml, the concentration of the recombinant human TGFβ (Cell signaling Technology, #8915LC) was 10 ng/ml, and the concentration of the antibody to be tested was 30 μg/ml.

The cover and filter membrane of the ChemoTx ® Disposable Chemotaxis System (Neuro Probe, #106-8) were opened, and the TGFβ-antibody mixture was added into the lower chamber, 30 μl per well, 4 pairs of wells for each group. The filter membrane was covered, and the cell-antibody mixture was added onto the membrane, 50 μl per well, 4 pairs of wells for each group. The cover was closed and placed in an incubator (37 ° C., 5% CO₂).

48 hours after incubation, the liquid on the filter membrane was removed with a clean paper towel, the filter membrane was opened, 10 μl of pre-cooled 0.25% trypsin was added to each well into the lower chamber, and the filter membrane was covered. 5 minutes after digestion, the samples were centrifuged at 1000 rpm for 1 minute. The cells which were adherent to the bottom of the plate were counted under an inverted microscope (Leica, #DMIL LED). Data were analyzed and processed with Graphpad Prism 6. Inhibition rate=[(average value of TGFβ−average value of sample group)/(average value of TGFβ group−average value of untreated group)]×100%

The experimental results are shown in Table 19.

TABLE 19
Results showing the in vitro inhibitory effect
of CTGF antibody on migration of PANC1 cells

| | Inhibition percentage of PANC1 cell |
|---|---|
| Test sample | migration in vitro (%) |
| Hu147-11wk | 73.9% |
| Hu147-33wk | 77.0% |
| Hu164-57wl | 81.2% |
| Hu164-67wl | 75.7% |
| Hu164-67yl | 78.0% |
| Hu95-51yk | 80.0% |
| Hu164-77wl | 74.3% |
| Hu164-87wl | 82.1% |
| Hu164-68wl | 76.9% |
| Hu164-69wl | 76.9% |
| mAb1 | 71.7% |

The results show that the humanized anti-CTGF antibodies of the present disclosure all can inhibit the migration ability of PANC1 cells in vitro induced by TGFβ1.

### Example 7. Assay of CTGF Antibody for In Vitro Inhibitory Effect on the Proliferation of PANC1 on Soft Agar

The deionized water was added into the agar (BD, #214220) and heated to prepare a 1.4% gel solution. 2×DMEM medium (Thermo, #12100046) was mixed with the gel solution in a ratio of 1:1 by volume, 500 μl was added to each well of a 24-well plate (Costar, #3524), and placed at 4 degrees for solidification.

PANC-1 cells (human pancreatic cancer cells, ATCC, #CRL-1469) were digested with 0.25% trypsin (Gibco, #25200-072), centrifuged and adjusted with DMEM medium (GE, #SH30243.01) containing 4% fetal bovine serum (Gibco, #10099-141) to make the cell density 5×10⁴ cells/ml. 2 mg/ml antibody was prepared with 2×DMEM medium. The cells, antibody and 1.4% gel solution were mixed at a ratio of 2:1:1 by volume, and 200 μl was added to each well of a 24-well plate covered with a lower layer of gel. After the upper gel has solidified, 200 μl of DMEM medium containing 2% fetal bovine serum was added to the 24-well plate. 28 days after incubation in an incubator (37° C., 5% CO₂), a high-content analysis system (Molecular Devices, ImageXpress) was used to develop imaging, and the area of the formed cell clone was analyzed. Inhibition rate=(1−average value of sample group/average value of untreated group)×100%. The experimental results are shown in Table 20.

TABLE 20
Results showing the inhibitory effect of CTGF antibody on
the proliferation of PANC1 cells on soft agar in vitro

| | Inhibition percentage of the in vitro |
|---|---|
| Test sample | proliferation of PANC1 cells (%) |
| Hu147-11wk | 23.76% |
| Hu147-33wk | 22.30% |
| Hu164-67wl | 24.2% |
| Hu164-67yl | 21.7% |
| Hu95-51yk | 20.24% |

The results show that the humanized antibodies derived from mAb147 and mAb164 clones have a significant inhibitory effect on the proliferation of human pancreatic cancer cells (PANC-1 cells) in vitro.

### Example 8: In Vivo Biological Activity of CTGF Antibody in Animals

### I. Assay of CTGF antibody for its inhibitory effect on mouse pulmonary fibrosis induced by bleomycin

Experimental protocol: 40 mice were randomly divided into the following 4 groups according to the body weight: normal control group (PBS, i.p., qod), mAb1 group (10 mg/kg, i.p., qod), Hu164-67wl (10 mg/kg, i.p., qod) group and Hu164-67yl (10 mg/kg, i.p., qod) group. There are 10 animals in each group, and the specific grouping is shown in Table 21 below. On day 1 of the experiment, each of the solvents and antibodies was intraperitoneally injected according to the body weight (10 ml/kg, normal control and model groups were provided with PBS); 2 hours later, aerosol bleomycin was used (50 mg/8 ml nebulization for 30 minutes and holding for 5 minutes) to establish models. Then, solvent or antibody was intraperitoneally injected every other day according to the above groups. The experimental period was 21 days, with a total of 11 intraperitoneal administrations. On day 22 of the experiment, the mice were anesthetized by intraperitoneal injection of 1% sodium pentobarbital (10 ml/kg), and then were fixed on the operating table on supine position. A 1 cm skin incision was made along the midline of the neck, and the lower end of the incision reached the entrance of chest. Forceps was used to bluntly separate the subcutaneous connective tissues and muscles to expose the trachea. The connective tissues on both sides of the trachea and between the trachea and the esophagus were separated to isolate the trachea free. A venous indwelling needle was inserted, and fixed with sutures by tying at a place where the cannula entering into the trachea. A 1 ml syringe filled with 0.8 ml PBS was connected to the inlet end of the venous indwelling needle, the PBS was slowly injected into the trachea, stayed for 30 seconds, and then the PBS was slowly withdrawn back. A milky foamy liquid could be observed during withdrawal. The lavage was repeated for three times, the lavage fluid was transfered into EP tubes, and then 0.5 ml PBS was taken, the above steps were repeated. The two lavage fluids were mixed and centrifuged at 1500 rpm for 5 min, and the supernatant was stored at −20° C. for testing. The BALF supernatant was centrifuged again at 10000 rpm for 10 min and the supernatant was taken to detect the soluble collagen (kit: Biocolor, Batch No. AA883).

TABLE 21
Test grouping and dosing regimen

| | Number of | | Dose | Route of | Frequency of |
|---|---|---|---|---|---|
| Group | animals | Agent | (mg/ml) | administration | administration |
| 1 | 10 | PBS | — | i.p. | Once every other day |
| 2 | 10 | mAb1 | 10 | i.p. | Once every other day |
| 3 | 10 | Hu164-67wl | 10 | i.p. | Once every other day |
| 4 | 10 | Hu164-67yl | 10 | i.p. | Once every other day |

Excel statistical software was used: the average value was calculated as avg (Average); SD (Standard Diviation) value was calculated as STDEV (Standard Diviation); SEM (Standard Error of Mean; Standard Deviation of the Sample Mean) value was calculated as STDEV/SQRT (Square root) (number of animals in each group); GraphPad Prism software was used for plotting, Two-way ANOVA (two-way analysis of variance) or One-way ANOVA (one-way analysis of variance) was used for statistical analysis of data.

The experimental results are shown in Table 22.

TABLE 22
Experimental result showing the inhibitory effect of CTGF
antibody on mouse pulmonary fibrosis induced by bleomycin

| | mAb1 | Hu164-67wl | Hu164-67yl |
|---|---|---|---|
| Agent | (10 mpk) | (10 mpk) | (10 mpk) |
| Increased | 53.2% | 72.8% (p < 0.05) | 80.7% (p < 0.05) |
| collagen | | | |
| inhibition rate | | | |

The results show that Hu164-67wl or Hu164-67yl show a strong inhibitory effect on pulmonary fibrosis in mice.

### II. Assay of CTGF Antibody for Inhibiting the Subcutaneously Transplanted Tumor of Su86.86 Human Pancreatic Cancer Cell

Experimental protocol: 200 μl of SU86.86 cells (ATCC, CRL-1837, 3.0×10⁶ cells) were inoculated subcutaneously into the right ribs of Nu/Nu nude mice. 11 days after the inoculation, when the tumor volume was about 140mm³, the mice with too high or low small body weight or tumor volume were excluded. The mice were randomly divided into 5 groups according to the tumor volume, 10 mice per group, and the administration was started on the same day. The antibody was injected intraperitoneally, twice a week, for a total of 18 days. The tumor volume and body weight were measured 1-2 times per week, and the data were recorded. Grouping and dosing regimen are shown in Table 23.

TABLE 23
Test grouping and dosing regimen

| | Number of | | Dose | Route of | Frequency of |
|---|---|---|---|---|---|
| Group | animals | Agent | (mg/ml) | administration | administration |
| 1 | 10 | Human IgG | 40 | i.p. | twice a week |
| 2 | 10 | mAb1 | 40 | i.p. | twice a week |
| 3 | 10 | Hu164-67yl | 40 | i.p. | twice a week |
| 4 | 10 | Hu164-67yl | 20 | i.p. | twice a week |
| 5 | 10 | Hu147-33wk | 40 | i.p. | twice a week |

Excel statistical software was used: the average value was calculated as avg; SD value was calculated as STDEV; SEM value was calculated as STDEV/SQRT (number of animals in each group); GraphPad Prism software was used for plotting, Two-way ANOVA or One-way ANOVA was used for statistical analysis of data.

The tumor volume (V) was calculated according to the following formula:

V=1/2×L_{long}×Lₛₕₒᵣₜ²

Relative tumor proliferation rate T/C (%)=(Tₜ−T₀)/(Cₜ−C₀)×100, where Tₜ and Cₜ are the tumor volumes of the treatment group and the control group at the end of the experiment; T₀ and C₀ are the tumor volume at the beginning of the experiment.

Tumor inhibition rate TGI (%)=1−T/C (%).

Experimental results:

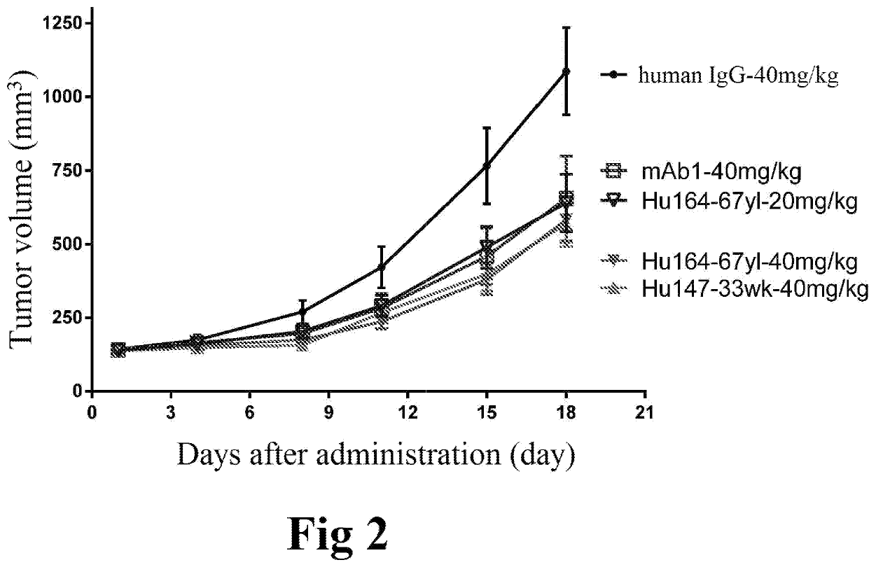

From D1 to D18, subcutaneous injection was performed twice a week, and the effect of CTGF antibodies on inhibiting the growth of SU86.86 tumor was detected. The experimental results are shown in Table 24 and FIG. 2. Compared with the blank control group of the same isotype human IgG, the tumor inhibition rates of mAb1-40mg/kg, Hu164-67yl-40mg/kg, Hu164-67yl-20 mg/kg and Hu147-33 wk (40mg/kg) group were 45%, 53%, 47% and 54%, respectively. mAb1-40mg/kg, Hu164-67yl-40mg/kg, Hu164-67yl -20mg/kg and Hu147-33 wk (40mg/kg) groups can significantly inhibit the growth of Su86.86 tumors (p<0.001), and the inhibitory effect on SU86.86 tumor is dose-dependent. In addition, the body weight of mice in each group did not change significantly.

TABLE 24
Experimental results showing the inhibitory effect
of CTGF antibody on SU86.86 xenograft in mice

| | | | | | % Tumor | |
|---|---|---|---|---|---|---|
| | Average tumor | | Average tumor | | inhibition | P (vs |
| | volume (mm³) | | volume (mm³) | | rate | blank |
| Grouping | D 0 | SEM | D 18 | SEM | D 18 | control) |
| Human IgG | 144.35 | 12.30 | 1087.10 | 148.21 | — | |
| mAb1 | 141.18 | 11.72 | 655.75 | 144.46 | 45 | p < 0.001 |
| (40 mg/kg) | | | | | | |
| Hu164-67yl | 138.75 | 8.71 | 582.95 | 76.14 | 53 | p < 0.001 |
| (40 mg/kg) | | | | | | |
| Hu164-67yl | 139.87 | 9.33 | 640.26 | 96.70 | 47 | p < 0.001 |
| (20 mg/kg) | | | | | | |
| Hu147-33wk | 138.00 | 10.11 | 569.66 | 77.40 | 54 | p < 0.001 |
| (40 mg/kg) | | | | | | |

1. An anti-CTGF antibody comprising a heavy chain variable region and a light chain variable region, wherein:
i) the heavy chain variable region comprises the same HCDR1, HCDR2 and HCDR3 sequences as those of the heavy chain variable region shown in SEQ ID NO: 8, and the light chain variable region comprises the same LCDR1, LCDR2 and LCDR3 sequences as those of the light chain variable region shown in SEQ ID NO: 9;
ii) the heavy chain variable region comprises the same HCDR1, HCDR2 and HCDR3 sequences as those of the heavy chain variable region shown in SEQ ID NO: 6, and the light chain variable region comprises the same LCDR1, LCDR2 and LCDR3 sequences as those of the light chain variable region shown in SEQ ID NO: 7;
ii-i) the heavy chain variable region comprises the same HCDR1, HCDR2 and HCDR3 sequences as those of the heavy chain variable region shown in SEQ ID NO: 69, and the light chain variable region comprises the same LCDR1, LCDR2 and LCDR3 sequences as those of the light chain variable region shown in SEQ ID NO: 70; or
ii-ii) the heavy chain variable region comprises the same HCDR1, HCDR2 and HCDR3 sequences as those of the heavy chain variable region shown in SEQ ID NO: 85, and the light chain variable region comprises the same LCDR1, LCDR2 and LCDR3 sequences as those of the light chain variable region shown in SEQ ID NO: 70.

2. The anti-CTGF antibody according to claim 1, comprising a heavy chain variable region and a light chain variable region, wherein:
iii) the heavy chain variable region comprises HCDR1, HCDR2 and HCDR3 as shown in SEQ ID NO: 16, SEQ ID NO: 17 and SEQ ID NO: 18, respectively, and the light chain variable region comprises LCDR1, LCDR2 and LCDR3 as shown in SEQ ID NO: 19, SEQ ID NO: 20 and SEQ ID NO: 21, respectively;
iv) the heavy chain variable region comprises HCDR1, HCDR2 and HCDR3 as shown in SEQ ID NO: 10, SEQ ID NO: 11 and SEQ ID NO: 12, respectively, and the light chain variable region comprises LCDR1, LCDR2 and LCDR3 as shown in SEQ ID NO: 13, SEQ ID NO: 14 and SEQ ID NO: 15, respectively;
iv-i) the heavy chain variable region comprises HCDR1, HCDR2 and HCDR3 as shown in SEQ ID NO: 71, SEQ ID NO: 72 and SEQ ID NO: 73, respectively, and the light chain variable region comprises LCDR1, LCDR2 and LCDR3 as shown in SEQ ID NO: 74, SEQ ID NO: 75 and SEQ ID NO: 76, respectively; or
iv-ii) the heavy chain variable region comprises HCDR1, HCDR2 and HCDR3 as shown in SEQ ID NO: 102, SEQ ID NO: 103 and SEQ ID NO: 104, respectively, and the light chain variable region comprises LCDR1, LCDR2 and LCDR3 as shown in SEQ ID NO: 74, SEQ ID NO: 75 and SEQ ID NO: 76, respectively.

3. The anti-CTGF antibody according to claim 1, wherein the anti-CTGF antibody is a murine antibody, a chimeric antibody or a humanized antibody.

4. The anti-CTGF antibody according to claim 1, comprising a heavy chain variable region and a light chain variable region, wherein:
(v) the amino acid sequence of the heavy chain variable region has at least 90% sequence identity to SEQ ID NO: 8, 33, 34, 35, or 36, and the amino acid sequence of the light chain variable region has at least 90% sequence identity to SEQ ID NO: 9, 30, 31 or 32;
(vi) the amino acid sequence of the heavy chain variable region has at least 90% sequence identity to SEQ ID NO: 6, 27, 28, or 29, and the amino acid sequence of the light chain variable region has at least 90% sequence identity to SEQ ID NO: 7, 22, 23, 24, 25 or 26; or
(vi-i) the amino acid sequence of the heavy chain variable region has at least 90% sequence identity to SEQ ID NO: 69, 81, 82, 83, 84, or 85, and the amino acid sequence of the light chain variable region has at least 90% sequence identity to SEQ ID NO: 70, 77, 78, 79, or 80.

5. The anti-CTGF antibody according to claim 2, wherein the anti-CTGF antibody is a humanized antibody;
the humanized antibody comprises a light chain variable region and a heavy chain variable region selected from the group consisting of the following (a), (b), (b-i) or (b-ii):
(a) a light chain variable region comprising:
LCDR1, LCDR2 and LCDR3 as shown in SEQ ID NO: 19, SEQ ID NO: 20 and SEQ ID NO: 21, respectively, and one or more back mutation(s) selected from the group consisting of 36V, 44F, 46G and 49G, and
a heavy chain variable region comprising:
HCDR1, HCDR2 and HCDR3 as shown in SEQ ID NO: 16, SEQ ID NO: 17 and SEQ ID NO: 18, respectively, and one or more back mutation(s) selected from the group consisting of 44G, 49G, 27F, 48L, 67L, 71K, 78V and 80F;
(b) a light chain variable region comprising:
LCDR1, LCDR2 and LCDR3 as shown in SEQ ID NO: 13, SEQ ID NO: 14 and SEQ ID NO: 15, respectively, and one or more back mutation(s) selected from the group consisting of 4L, 36F, 43S, 45K, 47W, 58V and 71Y, and
a heavy chain variable region comprising:
HCDR1, HCDR2 and HCDR3 as shown in SEQ ID NO: 10, SEQ ID NO: 11 and SEQ ID NO: 12, respectively, and one or more back mutation(s) selected from the group consisting of 28S, 30N, 49A, 75E, 76S, 93V, 94E and 104D a light chain variable region comprising:
(b-i) a light chain variable region comprising:
LCDR1, LCDR2 and LCDR3 as shown in SEQ ID NO: 74, SEQ ID NO: 75 and SEQ ID NO: 76, respectively, and one or more back mutation(s) selected from the group consisting of 45P, 46W, 48Y, 69S and 70Y, and
a heavy chain variable region comprising:
HCDR1, HCDR2 and HCDR3 as shown in SEQ ID NO: 71, SEQ ID NO: 72 and SEQ ID NO: 73, respectively, and one or more back mutation(s) selected from the group consisting of 27F, 38K, 481, 67K, 68A, 70L and 72F; or
(b-ii) a light chain variable region comprising:
LCDR1, LCDR2 and LCDR3 as shown in SEQ ID NO: 74, SEQ ID NO: 75 and SEQ ID NO: 76, respectively, and one or more back mutation(s) selected from the group consisting of 45P, 46W, 48Y, 69S and 70Y, and
a heavy chain variable region comprising:
HCDR1, HCDR2 and HCDR3 as shown in SEQ ID NO: 102, SEQ ID NO: 103 and SEQ ID NO: 104, respectively, and one or more back mutation(s) selected from the group consisting of 27F, 38K, 481, 67K, 68A, 70L and 72F.

6. The anti-CTGF antibody according to claim 5, comprising a heavy chain variable region and a light chain variable region, wherein:
(vii) the amino acid sequence of the heavy chain variable region is as shown in SEQ ID NO: 6, and the amino acid sequence of the light chain variable region is as shown in SEQ ID NO: 7;
(viii) the amino acid sequence of the heavy chain variable region is as shown in SEQ ID NO: 27, 28 or 29, and the amino acid sequence of the light chain variable region is as shown in SEQ ID NO: 22, 23, 24, 25 or 26;
(ix) the amino acid sequence of the heavy chain variable region is as shown in SEQ ID NO: 8, and the amino acid sequence of the light chain variable region is as shown in SEQ ID NO: 9;
(x) the amino acid sequence of the heavy chain variable region is as shown in SEQ ID NO: 33, 34, 35 or 36, and the amino acid sequence of the light chain variable region is as shown in SEQ ID NO: 30, 31 or 32;
(xi) the amino acid sequence of the heavy chain variable region is as shown in SEQ ID NO: 69, and the amino acid sequence of the light chain variable region is as shown in SEQ ID NO: 70; or
(xii) the amino acid sequence of the heavy chain variable region is as shown in SEQ ID NO:
81, 82, 83, 84, or 85, and the amino acid sequence of the light chain variable region is as shown in SEQ ID NO: 77, 78, 79 or 80.

7. The anti-CTGF antibody according to claim 1, wherein the antibody further comprises an antibody heavy chain constant region and a light chain constant region.

8. The anti-CTGF antibody according to any one of claim 1, comprising:
(c) a heavy chain having at least 85% sequence identity to SEQ ID NO: 41, 43, 44, 45, 46, 47, or 48, and a light chain having at least 85% sequence identity to SEQ ID NO: 42, 49, 50, 51, 52 or 53;
(d) a heavy chain having at least 85% sequence identity to SEQ ID NO: 54, 56, 57, 58, 59, 60, 61, 62, or 63, and a light chain having at least 85% sequence identity to SEQ ID NO: 55, 64, 65 or 66;
(e) a heavy chain as shown in SEQ ID NO: 41, 43, 44, 45, 46, 47 or 48, and a light chain as shown in SEQ ID NO: 42, 49, 50, 51, 52 or 53;
(f) a heavy chain as shown in SEQ ID NO: 54, 56, 57, 58, 59, 60, 61, 62 or 63, and a light chain as shown in SEQ ID NO: 55, 64, 65 or 66;
(g) a heavy chain having at least 85% sequence identity to SEQ ID NO: 86, 88, 89, 90, 91, 92, 93, 94, 95, 96, or 97, and a light chain having at least 85% sequence identity to SEQ ID NO: 87, 98, 99, 100 or 101; or
(h) a heavy chain as shown in SEQ ID NO: 86, 88, 89, 90, 91, 92, 93, 94, 95, 96 or 97, and a light chain as shown in SEQ ID NO: 87, 98, 99, 100 or 101.

9. An isolated anti-CTGF antibody, wherein the isolated anti-CTGF antibody competes for the binding to human CTGF with the anti-CTGF antibody of any one of claim 1.

10. A nucleic acid molecule encoding the anti-CTGF antibody of any one of claim 1.

11. A host cell comprising the nucleic acid molecule of claim 10.

12. A pharmaceutical composition comprising a therapeutically effective amount of the anti-CTGF antibody according to claim 1, and one or more pharmaceutically acceptable carriers, diluents, buffers or excipients.

13. A method for the immunoassay or determination of CTGF, the method comprising a step of contacting the anti-CTGF antibody of claim 1 with a subject or a sample thereof.

14. A kit comprising the anti-CTGF antibody according to claim 1.

15. A method for the treatment or prevention of CTGF-related disease, the method comprising administering to a subject a therapeutically effective amount or a prophylactically effective amount of the anti-CTGF antibody according to claim 1.

16. The anti-CTGF antibody according to claim 6, comprising a heavy chain variable region and a light chain variable region, wherein:
(xiii) the amino acid sequence of the heavy chain variable region is as shown in SEQ ID NO: 34, and the amino acid sequence of the light chain variable region is as shown in SEQ ID NO: 30;
(xiv) the amino acid sequence of the heavy chain variable region is as shown in SEQ ID NO: 27, and the amino acid sequence of the light chain variable region is as shown in SEQ ID NO: 22; or
(xv) the amino acid sequence of the heavy chain variable region is as shown in SEQ ID NO: 85, and the amino acid sequence of the light chain variable region is as shown in SEQ ID NO: 77.

17. The anti-CTGF antibody according to claim 1, comprising:
(j) a heavy chain as shown in SEQ ID NO: 61, and a light chain as shown in SEQ ID NO: 64;
(k) a heavy chain as shown in SEQ ID NO: 46, and a light chain as shown in SEQ ID NO: 49; or
(l) a heavy chain as shown in SEQ ID NO: 97, and a light chain as shown in SEQ ID NO: 98.
